SEQUENCE LISTING (3 sequences)

SEQ ID NO: 1 - DNA, 26 bp, Artificial Sequence
  primer
ataacctaagaactggcccataactc

SEQ ID NO: 2 - DNA, 19 bp, Artificial Sequence
  primer
cggccaaggattgaagtca

SEQ ID NO: 3 - DNA, 34 bp, Artificial Sequence
  primer
attgattacgaaaaatggaataaccaccaaagga

(19) United States
(12) Patent Application Publication
(10) Pub. No.: US 2006/0018875 A1
(43) Pub. Date: Jan. 26, 2006

(54) Interferon compositions and methods of use thereof

(76) Inventors: Lawrence M. Blatt, San Francisco, CA (US); Ramachandran Radhakrishnan, Fremont, CA (US); Scott D. Seiwert, Pacifica, CA (US); Hua Tan, Daly City, CA (US); Jena Rey Derrick, San Francisco, CA (US); Jin Hong, Brisbane, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/153,864

(22) Filed: Jun. 14, 2005

Related U.S. Application Data
(60) Provisional application No. 60/579,625, filed on Jun. 14, 2004.

(51) Int. Cl. A61K 38/21

(52) U.S. Cl. 424/85.6; 424/85.7

(57) ABSTRACT

The present invention provides compositions, including pharmaceutical compositions, comprising IFN-α and IFN-γ. The present invention further provides methods of treating various disorders, the methods comprising administering an effective amount of a subject composition.

### CROSS-REFERENCE

This application claims the benefit of U.S. Provisional Patent Application No. 60/579,625, filed Jun. 14, 2004, which application is incorporated herein by reference in its entirety.

### FIELD OF THE INVENTION

The present invention is in the field of cytokine compositions, and in particular compositions comprising IFN-γ and IFN-α, and use of the compositions to treat viral infections, fibrotic disorders, and proliferative disorders.

### BACKGROUND OF THE INVENTION

Interferons are a family of cytokines that provide one of the most potent host defense mechanisms against viral infections. The levels and expression of endogenous interferons can be altered when there is an infectious disease and/or during therapeutic trials and several endogenous interferons can synergistically act and so, improve their antiviral protection potential.

The naturally-occurring interferons that have been described to date have been classified into two types. Type I interferons include IFN-α (formerly known as leukocyte interferon); IFN-β (formerly known as fibroblast interferon); IFN-ω; IFN-tau; and IFN-κ. Type II interferons include IFN-γ. All interferons, including alpha (IFN-α) and gamma (IFN-γ), have potent antiviral activity. However, the main biological activity of IFN-gamma appears to be immunomodulatory in contrast to the other interferons, which exhibit primarily antiviral activity.

There is a need in the art for combination interferon formulations suitable for treating various disorders. The present invention addresses this need.

Literature

U.S. Pat. Nos. 6,479,049; 5,252,714; 5,382,657; 5,539,063; 5,559,213; 5,672,662; 5,747,646; 5,766,581; 5,792,834; 5,795,569; 5,798,232; 5,824,784; 5,834,594; 5,849,860; 5,928,636; 5,951,974; 5,595,732; 5,981,709; 5,985,265; 6,005,075; 6,180,096; 6,250,469; 6,277,830.

### SUMMARY OF THE INVENTION

The present invention provides compositions, including pharmaceutical compositions, comprising IFN-α and IFN-γ, wherein the compositions are prepared by admixing a composition comprising IFN-α and a composition comprising IFN-γ. The present invention further provides methods of preparing a subject composition. The present invention further provides containers and kits comprising a subject composition. The present invention further provides methods of treating various disorders, the methods comprising administering an effective amount of a subject composition.

### BRIEF DESCRIPTION OF THE DRAWINGS

FIG. 1 is a graph depicting the results of an SEC-HPLC chromatographic analysis of the stability retention of an Infergen/Actimmune mixture incubated for 0 hour (hr), 2 hours (hrs), 4 hrs, or 8 hrs at ambient temperature. The data are presented as chromatographic overlays of the SEC-HPLC traces for the individual incubation times.

FIG. 2 is a graph depicting the results of an RP-HPLC chromatographic analysis of the stability retention of an Infergen/Actimmune mixture incubated for 0 hr, 2 hrs, 4 hrs or 8 hrs at ambient temperature. The data are presented as chromatographic overlays of the RP-HPLC traces for the individual incubation times.

FIG. 3 is a graph depicting the bioactivity retention of Infergen/Actimmune mixtures incubated for 0 hr, 2 hrs, 4 hrs, 8 hrs or 24 hours at ambient temperature. Bioactivity data are presented as EC50 values measured in L-EMC virus/A-549 cell cytopathic effect inhibition assays.

FIG. 4 is a graph depicting the effect on viral replication of Infergen and of Actimmune alone or in combination in a dengue virus cell-based assay model.

FIG. 5 is a graph depicting the combination index of two different combinations of Infergen and Actimmune (1:120 and 1:60) for several effective doses.

FIG. 6 is a graph depicting the expression ratios of several different antiviral genes in a gene set from hepatocytes exposed to Infergen alone or to a combination of Infergen and Actimmune.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans.

Drug delivery devices that are suitable for use in the subject methods include, but are not limited to, injection devices; an implantable device, e.g., pumps, such as an osmotic pump, that may or may not be connected to a catheter; biodegradable implants; liposomes; depots; and microspheres.

The terms “controlled drug delivery device” and “controlled delivery device” are used interchangeably herein to refer to any device wherein (i) the release (e.g., rate, timing of release) of a drug or other desired substance contained therein is controlled by or determined by the device itself and not substantially influenced by the environment of use, and (ii) the release of (i) occurs at a rate that is reproducible within the environment of use.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a depot comprising an antiviral agent; installation of a continuous delivery device (e.g., a pump or other controlled release injectible system); and a single subcutaneous injection followed by installation of a continuous delivery system.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

The terms “International Units” and “Units” are used interchangeably herein to refer to units of measurement for quantitation of the ability of the interferon to inhibit the cytopathic effect of a suitable virus (e.g. encephalomyocarditis virus (EMC), vesicular stomatitis virus, Semliki forest virus) after infection of an appropriate cell line (e.g., the human lung carcinoma cell lines, A549; HEP2/C; and the like). The antiviral activity is normalized to “Units” of antiviral activity exhibited by a reference standard such as human interferon alpha supplied by the World Health Organization (WHO). Such methods are detailed in numerous references. A particular method for measuring International Units is described in Familletti, P. C., Rubinstein, S and Pestka, S. (1981) “A convenient and rapid cytopathic effect inhibition assay for interferon”, Methods in Enzymol, Vol 78 (S. Pestka, ed), Academic Press, New York pages 387-394. For the most part, the reference standard is human interferon alpha supplied by the WHO, and the method for measuring International Units is that described in Familletti, supra.

The amounts of interferon administered will depend on the specific activities of the compounds and their biological performance in vivo. For example, IFN-α 2b is administered at 11.54 μg protein three times a week corresponding to 3×106 IU per injection (specific activity, 2.68×106 IU/mg). On the other hand, CIFN alfa-con 1 is administered at 9 μg doses per injection corresponding to 9×106 IU per administration (specific activity, 1×109 IU/mg). However, in view of the fact that PEGylation reactions often result in a reduction in activity, larger mass doses of PEGylated material are administered to achieve efficacy (e.g. reduction in viral load; sustained viral response, etc.).

As used herein, an “admixture” of IFN-α and IFN-γ, a pharmaceutical product or composition made by “admixing” a pharmaceutical composition of IFN-α and a pharmaceutical composition of IFN-γ, and any language of similar meaning, refers to a mixture formed by (1) admixing a workable liquid pharmaceutical formulation of IFN-α with a workable liquid pharmaceutical formulation of IFN-γ (2) reconstituting or solubilizing a workable lyophilized pharmaceutical formulation of IFN-α in a workable liquid pharmaceutical formulation of IFN-γ or (3) reconstituting or solubilizing a workable lyophilized pharmaceutical formulation of IFN-γ in a workable liquid pharmaceutical formulation of IFN-α.

As used herein, the terms “combination pharmaceutical composition” of IFN-α and IFN-γ, or language of similar meaning, are meant to be synonymous with “admixture” of IFN-α and IFN-γ.

As used herein, any compound or agent described as “effective for the avoidance or amelioration of side effects induced by an IFN-α and/or an IFN-γ,” or as “effective for reducing or eliminating the severity or occurrence of side effects induced by an IFN-α and/or an IFN-γ,” or any compound or agent described by language with a meaning similar or equivalent to that of either of the foregoing quoted passages, is/are defined as a compound(s) or agent(s) that when co-administered to a patient in an effective amount along with a given dosing regimen of a subject an IFN-α/IFN-γ combination therapy, abates or eliminates the severity or occurrence of side effects experienced by a patient in response to the given dosing regimen of the IFN-α/IFN-γ combination therapy, as compared to the severity or occurrence of side effects that would have been experienced by the patient in response to the same dosing regimen of the IFN-α/IFN-γ combination therapy without co-administration of the agent.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectable system); and a single subcutaneous injection followed by installation of a continuous delivery system.

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of ”substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

The terms “fibrotic condition,” “fibrotic disease,” and “fibrotic disorder” are used interchangeably to refer to a condition, disease or disorder that is amenable to treatment by administration of a compound having anti-fibrotic activity. Fibrotic disorders include, but are not limited to, pulmonary fibrosis, including idiopathic pulmonary fibrosis (IPF) and pulmonary fibrosis from a known etiology, liver fibrosis, and renal fibrosis. Other exemplary fibrotic conditions include musculoskeletal fibrosis, cardiac fibrosis, post-surgical adhesions, scleroderma, glaucoma, and skin lesions such as keloids.

The terms “proliferative disorder” and “proliferative disease” are used interchangeably to refer to any disease or condition characterized by pathological cell growth or proliferation, particularly cancer.

The terms “cancer,” “neoplasm,” and “tutor,” are used interchangeably herein to refer to cells which exhibit relatively autonomous growth, so that they exhibit an aberrant growth phenotype characterized by a significant loss of control of cell proliferation. Cancerous cells can be benign or malignant.

The term “hepatitis virus infection” refers to infection with one or more of hepatitis A, B, C, D, or E virus, with blood-borne hepatitis viral infection being of particular interest, particularly hepatitis C virus infection.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “chemotherapeutic agent” or “chemotherapeutic” (or “chemotherapy”, in the case of treatment with a chemotherapeutic agent) is meant to encompass any non-proteinaceous (i.e., non-peptidic) chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelaamines including altretamine, triethylenemelaamine, triethylenephosphoramide, triethylenethiophosphoramide and trimethylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g. calicheamicin, especially calicheamicin gamma1I and calicheamicin phiI12, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubincin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as amninoglutethimide, mitotane, trilostane; folic acid replinisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g. paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzarm); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluromethylomithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

The term “antineoplastic” agent, drug or compound is meant to refer to any agent, including any chemotherapeutic agent, biological response modifier (including without limitation (i) proteinaceous, i.e. peptidic, molecules capable of elaborating or altering biological responses and (ii) non-proteinaceous, i.e. non-peptidic, molecules capable of elaborating or altering biological responses), cytotoxic agent, or cytostatic agent, that reduces proliferation of a neoplastic cell.

The term “biological response modifier” refers to any proteinaceous (i.e., peptidic) molecule or any non-proteinaceous (i.e., non-peptidic) molecule capable of elaborating or altering a biological response relevant to the treatment of cancer. Examples of biological response modifiers include antagonists of tumor-associated antigens, such as anti-tumor antigen antibodies, antagonists of cellular receptors capable of inducing cell proliferation, agonists of cellular receptors capable of inducing apoptosis, such as Apo-2 ligands, Type I interferon receptor agonists, such as interferon-α molecules and interferon-β molecules, Type II interferon receptor agonists, such as interferon-γ molecules, Type III interferon receptor agonists, such as IL-28A, IL-28B, and IL-29, antagonists of inflammatory cytokines, including tumor necrosis factor (TNF) antagonists, such as anti-TNF antibodies (e.g. REMICADE™ anti-TNF monoclonal antibody) and soluble TNF receptor (e.g. ENBREL™TNF receptor-Ig immunoadhesin), growth factor cytokines, such as hematopoietic cytokines, including erythropoietins, such as EPOGEN™ epoetin-alfa, granulocyte colony stimulating factors (G-CSFs), such as NEUPOGEN™ filgrastim, granulocyte-macrophage colony stimulating factors (GM-CSFs), and thrombopoietins, lymphocyte growth factor cytokines, such as interleukin-2, and antagonists of growth factor cytokines, including antagonists of angiogenic factors, e.g. vascular endothelial cell growth factor (VEGF) antagonists, such as AVASTIN™ bevacizumab (anti-VEGF monoclonal antibody).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these small& ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an IFN-α polypeptide” includes a plurality of such polypeptides and reference to “the pharmaceutical composition” includes reference to one or more pharmaceutical compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides admixtures of IFN-α and IFN-γ pharmaceutical compositions, as well as containers and kits comprising the separate pharmaceutical components of the admixtures, i.e., the individual IFN-α pharmaceutical compositions and IFN-γ pharmaceutical compositions that are used to form the admixtures. The admixtures typically comprise amounts of IFN-α and IFN-γ that are effective to treat a disorder in an individual when administered as separate pharmaceutical compositions, i.e., when the individual receives IFN-α and IFN-γ in separate administrations (without any mixture of the two drugs prior to delivery). A subject admixture is useful for treating various disorders, including viral infections, fibrotic disorders, and proliferative disorders.

Compositions

The present invention provides pharmaceutical compositions comprising IFN-α and IFN-γ, wherein the pharmaceutical compositions are prepared by admixing a composition comprising an IFN-α polypeptide and a composition comprising an IFN-γ polypeptide. Typically, the separate components (e.g., a composition comprising IFN-α and a composition comprising IFN-γ) are sterile compositions and are suitable, as an admixture, for administration to a mammalian subject, e.g., a human subject. The separate compositions are generally pharmaceutical compositions, e.g., a subject composition comprises IFN-α or IFN-γ, and a pharmaceutically acceptable excipient. A wide variety of pharmaceutically acceptable excipients are known in the art and need not be discussed in detail herein.

In some embodiments, a subject IFN-α/IFN-γ combination pharmaceutical composition is prepared by a method comprising: admixing a) a first pharmaceutical composition comprising an IFN-α polypeptide and a first pharmaceutically acceptable excipient in a sterile aqueous solution; and b) a second pharmaceutical composition comprising an IFN-γ polypeptide and a second pharmaceutically acceptable excipient in a sterile aqueous solution; where the first and second pharmaceutically acceptable excipients are the same or different.

In some embodiments, where the IFN-α/IFN-γ combination pharmaceutical composition is prepared by admixing separate IFN-α and IFN-γ aqueous solutions, the admixing takes place in a syringe that includes two chambers, or barrels. For example, in some of these embodiments, the first pharmaceutical composition comprising an IFN-αpolypeptide in a sterile aqueous solution is in a first barrel of the syringe; and the second pharmaceutical composition comprising an IFN-γ polypeptide in a sterile aqueous solution is in the second barrel of the syringe; and the admixing is carried out by simultaneously introducing the contents of the first barrel and the contents of the second barrel into a common channel terminating in an aperture, e.g., a hypodermic needle, positioned in the body of the patient. The introduction of the IFN-α and IFN-γ solutions into the common channel effects admixture of the solutions, thereby forming the combination pharmaceutical composition prior to administration of the drugs to the patient.

A subject IFN-α/IFN-γ pharmaceutical composition is prepared by mixing any workable pharmaceutical formulation of IFN-α with any workable pharmaceutical formulation of IFN-γ. In some embodiments, a suitable workable pharmaceutical formulation is a sterile aqueous solution. In other embodiments, a suitable workable pharmaceutical formulation is a sterile lyophilized formulation.

In some embodiments, a subject combination pharmaceutical composition is prepared by a method comprising solubilizing or reconstituting a lyophilized preparation of an IFN-α polypeptide with a sterile aqueous solution comprising an IFN-γ polypeptide. In some of these embodiments, the solubilization takes place by admixing the contents of two chambers, where a first chamber contains a pharmaceutical composition comprising an IFN-γ polypeptide in a sterile aqueous solution, and where the second chamber contains a sterile, lyophilized composition comprising an IFN-α polypeptide. The combination IFN-α/IFN-γ pharmaceutical composition is formed by introducing the contents of the first chamber into the second chamber, wherein the lyophilized IFN-α polypeptide is solubilized or reconstituted in the first pharmaceutical composition comprising a sterile IFN-γ aqueous solution.

In some embodiments, a subject combination pharmaceutical composition is prepared by a method comprising solubilizing or reconstituting a lyophilized preparation of INFERGEN® consensus IFN-α with a sterile aqueous solution comprising an IFN-γ polypeptide. In some of these embodiments, the solubilization takes place by admixing the contents of two chambers, where a first chamber contains a pharmaceutical composition comprising an IFN-γ polypeptide in a sterile aqueous solution, and where the second chamber contains a sterile pharmaceutical composition comprising lyophilized INFERGEN® consensus IFN-α. The combination IFN-α/IFN-γ pharmaceutical composition is formed by introducing the contents of the first chamber into the second chamber, wherein the lyophilized INFERGEN® consensus IFN-α is solubilized or reconstituted in the first pharmaceutical composition comprising a sterile IFN-γ aqueous solution.

In some embodiments, a subject combination pharmaceutical composition is prepared by a method comprising solubilizing or reconstituting a lyophilized preparation of INFERGEN® consensus IFN-α with a sterile aqueous solution comprising Actimmune® human IFN-γ1b. In some of these embodiments, the solubilization takes place by admixing the contents of two chambers, where a first chamber contains a pharmaceutical composition comprising Actimmune® human IFN-γ1b in a sterile aqueous solution, and where the second chamber contains a sterile pharmaceutical composition comprising lyophilized INFERGEN® consensus IFN-α. The combination IFN-α/IFN-γ pharmaceutical composition is formed by introducing the contents of the first chamber into the second chamber, wherein the lyophilized INFERGEN® consensus IFN-α is solubilized or reconstituted in the first pharmaceutical composition comprising a sterile Actimmune® human IFN-γ1b aqueous solution.

In other embodiments, a subject combination pharmaceutical composition is prepared by a method comprising solubilizing or reconstituting a lyophilized preparation of an IFN-γ polypeptide with a sterile aqueous solution comprising an IFN-α polypeptide. In some of these embodiments, the solubilization takes place by admixing the contents of two chambers, where a first chamber contains a pharmaceutical composition comprising an IFN-α polypeptide in a sterile aqueous solution, and where the second chamber contains a sterile pharmaceutical composition comprising a lyophilized IFN-γ polypeptide. The combination IFN-α/IFN-γ pharmaceutical composition is formed by introducing the contents of the first chamber into the second chamber, wherein the lyophilized IFN-γ polypeptide is solubilized or reconstituted in the first pharmaceutical composition comprising a sterile IFN-α aqueous solution, to form the combination IFN-α/IFN-γ pharmaceutical composition.

In other embodiments, a subject combination pharmaceutical composition is prepared by a method comprising solubilizing or reconstituting a lyophilized preparation of Actimmune® human IFN-γ1b with a sterile aqueous solution comprising an IFN-α polypeptide. In some of these embodiments, the solubilization takes place by admixing the contents of two chambers, where a first chamber contains a pharmaceutical composition comprising an IFN-α polypeptide in a sterile aqueous solution, and where the second chamber contains a sterile pharmaceutical composition comprising lyophilized Actimmune® human IFN-γ1b. The combination IFN-α/IFN-γ pharmaceutical composition is formed by introducing the contents of the first chamber into the second chamber, wherein the lyophilized Actimmune® human IFN-γ1b is solubilized or reconstituted in the first pharmaceutical composition comprising a sterile IFN-α aqueous solution, to form the combination IFN-60 /IFN-γ pharmaceutical composition.

In other embodiments, a subject combination pharmaceutical composition is prepared by a method comprising solubilizing or reconstituting a lyophilized preparation of Actimmune® human IFN-γ1b with a sterile aqueous solution comprising INFERGEN® consensus IFN-α. In some of these embodiments, the solubilization takes place by admixing the contents of two chambers, where a first chamber contains a pharmaceutical composition comprising INFERGEN® in a sterile aqueous solution, and where the second chamber contains a sterile pharmaceutical composition comprising lyophilized Actimmune® human IFN-γ1b. The combination IFN-α/IFN-γ pharmaceutical composition is formed by introducing the contents of the first chamber into the second chamber, wherein the lyophilized Actimmune® human IFN-γ1b is solubilized or reconstituted in the first pharmaceutical composition comprising INFERGEN® in a sterile aqueous solution, to form the combination IFN-α/IFN-γ pharmaceutical composition.

A subject IFN-α/IFN-γ combination pharmaceutical composition is in some embodiments prepared upon use or up to about 1 second to about 1 month prior to use, e.g., upon use, or from about 1 second to about 5 seconds, from about 5 seconds to about 10 seconds, from about 10 seconds to about 15 seconds, from about 15 seconds to about 30 seconds, from about 30 seconds to about 60 seconds, from about 60 seconds to about 5 minutes, from about 5 minutes to about 15 minutes, from about 15 minutes to about 30 minutes, from about 30 minutes to about 1 hour, from about 1 hour to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, from about 72 hours to about 4 days, from about 4 days to about 7 days, from about 7 days to about 2 weeks, or from about 2 weeks to about one month prior to use (e.g., prior to administration to an individual in need thereof).

In some embodiments, the ratio of IFN-α to IFN-γ (on a polypeptide weight basis) in a subject admixture varies from about 1:1 to about 1:600, e.g., from about 1:1 to about 1:1.5, from about 1:1.5 to about 1:2, from about 1:2 to about 1:3, from about 1:3 to about 1:5, from about 1:5 to about 1:10, from about 1:10 to about 1:15, from about 1:15 to about 1:20, from about 1:20 to about 1:30, from about 1:30 to about 1:50, from about 1:50 to about 1:60, from about 1:50 to about 1:100, from about 1:60 to about 1:120, from about 1:100 to about 1:150, from about 1:150 to about 1:200, from about 1:200 to about 1:300, from about 1:300 to about 1:400, from about 1:400 to about 1:500, or from about 1:500 to about 1:600.

A subject admixture is stable over a period of time ranging from about 1 hour to about 1 month, e.g., from about 1 hour to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, from about 72 hours to about 4 days, from about 4 days to about 7 days, from about 7 days to about 2 weeks, or from about 2 weeks to about one month.

A subject admixture is “stable,” e.g., the IFN-α and IFN-γ polypeptides in a subject admixture each individually retain at least about 50%, at least about 60%, at least about 70%, at least about 80%, at least about 90%, at least about 95%, or at least about 98%, or more, of an activity of the IFN-α and IFN-γ polypeptides in a parent stock solution. In some embodiments, the activity of the IFN-α or IFN-γ polypeptide is an antiviral activity. Anti-viral activity is readily measured using an in vitro cell-based assay, e.g., as described in Example 1, below. In other embodiments, the activity of the IFN-α or IFN-γ polypeptide is an anti-proliferative activity. Anti-proliferative activity is readily measured, e.g., in an in vitro cell-based assay by determining the effect of the IFN-α or IFN-γ polypeptide on proliferation of a cell line, e.g., a human cell line.

A subject admixture is stable for a period of time ranging from about 1 hour to about 1 month, as noted above, when stored at a temperature ranging from about 4° C. to about 25° C., e.g., from about 4° C to about 7° C., from about 7° C. to about 10° C., from about 10° C. to about 15° C., from about 15° C. to about 17° C., from about 17° C. to 20° C., from about 20° C. to about 22° C., or from about 22° C. to about 25° C.

In pharmaceutical dosage forms, active agents (e.g., IFN-α and IFN-γ polypeptides) may be administered in their pharmaceutically acceptable forms, either alone or in appropriate association, as well as in combination, with other pharmaceutically active compounds. The following methods and excipients are merely exemplary and are in no way limiting.

The active agents (e.g., IFN-α and IFN-γ polypeptides) can be formulated into preparations for injection by dissolving, suspending or emulsifying them in an aqueous solvent (e.g., saline, and the like), or a nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms of the IFN-α and IFN-γ agents for injection or intravenous administration may comprise the respective agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an IFN-α or IFN-γ agent, calculated in an amount sufficient to produce the desired effect in association with a pharmaceutically acceptable diluent, carrier or vehicle. The specifications for the unit dosage forms depend, at least in part, on the particular IFN-α and IFN-γ polypeptides employed and the effect to be achieved, and the pharmacodynamics associated with the particular IFN-α and IFN-γ polypeptides in the host.

Effective Dosages

Effective dosages of an IFN-α can range from about 1 μg to about 30 μg, from about 3 μg to about 27 μg, from about 1 MU to about 20 MU, from about 3 MU to about 10 MU, from about 90 μg to about 180 μg, or from about 18 μg to about 90 μg.

Effective dosages of Infergen(V consensus IFN-α include about 3 μg, about 9 μg, about 15 μg, about 18 μg, or about 27 μg of drug per dose. Effective dosages of IFN-α2a and IFN-α2b can range from 3 million Units (MU) to 10 MU per dose. Effective dosages of PEGylated IFN-α2a can contain an amount of about 90 μg to 360 μg, or about 180 μg, of drug per dose. Effective dosages of PEGylated IFN-α2b can contain an amount of about 0.5 μg to 3.0 μg, or about 1.0 μg to 1.5 μg, of drug per kg of body weight per dose. Effective dosages of PEGylated consensus interferon (PEG-CIFN) can contain an amount of about 10 μg to about 100 μg, or from about 40 μg to about 80 μg, or about 45 μg to about 60 μg, of CIFN amino acid weight per dose of PEG-CIFN.

Effective dosages of IFN-γ can range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 50 μg, about 100 μg, or about 200 μg IFN-γ. In many embodiments of interest, IFN-γ1b is administered.

Where the dosage is 100 μg IFN-γ per dose, the amount of IFN-γ per body weight (assuming a range of body weights of from about 45 kg to about 135 kg) is in the range of from about 2.2 μg IFN-γ per kg body weight to about 0.74 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m².

In some embodiments, the dosage groups range from about 25 μg/m² to about 100 μg/m². In other embodiments, the dosage groups range from about 25 μg/m² to about 50 μg/m².

In some embodiments, the invention employs any of the above-described IFN-α dosages and any of the above-described IFN-γ dosages in an admixture for parenteral administration. In these embodiments, each of the IFN-α and IFN-γ compositions in the admixture is suitable for parenteral administration.

In some embodiments, the invention employs any of the above-described IFN-α dosages and any of the above-described IFN-γ dosages in an admixture for subcutaneous administration. In these embodiments, each of the IFN-α and IFN-γ compositions in the admixture is suitable for subcutaneous administration.

In one particular embodiment, a subject admixture comprises effective amounts of IFN-α and IFN-γ, wherein the admixture comprises monoPEG (30 kD, linear)-ylated consensus IFN-α in an amount of 100 μg and IFN-γ in an amount of from about 10 μg to about 300 μg.

In one particular embodiment, a subject admixture comprises effective amounts of IFN-γ and IFN-γ, wherein the admixture comprises monoPEG (30 kD, linear)-ylated consensus IFN-α in an amount of 150 μg and IFN-γ in an amount of from about 10 μg to about 300 μg.

In another particular embodiment, a subject admixture comprises effective amounts of IFN-γ and IFN-γ, wherein the admixture comprises monoPEG (30 kD, linear)-ylated consensus IFN-α in an amount of 200 μg and IFN-γ in an amount of from about 10 μg to about 300 μg.

In another particular embodiment, a subject admixture comprises effective amounts of IFN-α and IFN-γ, wherein the admixture comprises monoPEG (30 kD, linear)-ylated consensus IFN-α in an amount of 100 μg and Actimmune® IFN-γ in an amount of 50 μg.

In another particular embodiment, a subject admixture comprises effective amounts of IFN-α and IFN-γ, wherein the admixture comprises monoPEG (30 kD, linear)-ylated consensus IFN-α in an amount of 100 μg and Actimmune® IFN-γ in an amount of 100 μg.

In another particular embodiment, a subject admixture comprises effective amounts of IFN-α and IFN-γ, wherein the admixture comprises monoPEG (30 kD, linear)-ylated consensus IFN-α in an amount of 100 μg and Actimmune® IFN-γ in an amount of 150 μg.

In another particular embodiment, a subject admixture comprises effective amounts of IFN-α and IFN-γ, wherein the admixture comprises monoPEG (30 kD, linear)-ylated consensus IFN-α in an amount of 100 μg and Actimmune® IFN-γ in an amount of 200 μg.

In another particular embodiment, a subject admixture comprises effective amounts of IFN-α and IFN-γ, wherein the admixture comprises monoPEG (30 kD, linear)-ylated consensus IFN-α in an amount of 150 μg and Actimmune® IFN-γ in an amount of 50 μg.

In another particular embodiment, a subject admixture comprises effective amounts of IFN-α and IFN-γ, wherein the admixture comprises monoPEG (30 kD, linear)-ylated consensus IFN-α in an amount of 150 μg and Actimmune® IFN-γ in an amount of 100 μg.

In another particular embodiment, a subject admixture comprises effective amounts of IFN-α and IFN-γ, wherein the admixture comprises monoPEG (30 kD, linear)-ylated consensus IFN-α in an amount of 150 μg and Actimmune® IFN-γ in an amount of 150 μg.

In another particular embodiment, a subject admixture comprises effective amounts of IFN-α and IFN-γ, wherein the admixture comprises monoPEG (30 kD, linear)-ylated consensus IFN-α in an amount of 150 μg and Actimmune® IFN-γ in an amount of 200 μg.

In another particular embodiment, a subject admixture comprises effective amounts of IFN-α and IFN-γ, wherein the admixture comprises monoPEG (30 kD, linear)-ylated consensus IFN-α in an amount of 200 μg and Actimmune® IFN-γ in an amount of 50 μg.

In another particular embodiment, a subject admixture comprises effective amounts of IFN-α and IFN-γ, wherein the admixture comprises monoPEG (30 kD, linear)-ylated consensus IFN-α in an amount of 200 μg and Actimmune® IFN-γ in an amount of 100 μg.

In another particular embodiment, a subject admixture comprises effective amounts of IFN-α and IFN-γ, wherein the admixture comprises monoPEG (30 kD, linear)-ylated consensus IFN-α in an amount of 200 μg and Actimmune® IFN-γ in an amount of 150 μg.

In another particular embodiment, a subject admixture comprises effective amounts of IFN-α and IFN-γ, wherein the admixture comprises monoPEG (30 kD, linear)-ylated consensus IFN-α in an amount of 200 μg and Actimmune® IFN-γ in an amount of 200 μg.

In another particular embodiment, a subject admixture comprises effective amounts of IFN-α and IFN-γ, wherein the admixture comprises PEGASYS® PEGylated IFN-α2a in an amount of from about 90 μg to about 360 μg and IFN-γ in an amount of from about 10 μg to about 300 μg.

In another particular embodiment, a subject admixture comprises effective amounts of IFN-α and IFN-γ, wherein the admixture comprises PEGASYS® PEGylated IFN-α2a in an amount of about 180 μg and IFN-γ in an amount of from about 10 μg to about 300 μg.

In another particular embodiment, a subject admixture comprises effective amounts of IFN-α and IFN-γ, wherein the admixture comprises PEGASYS® PEGylated IFN-α2a in an amount of about 180 μg and Actimmune® IFN-γ in an amount of 50 μg.

In another particular embodiment, a subject admixture comprises effective amounts of IFN-α and IFN-γ, wherein the admixture comprises PEGASYS® PEGylated IFN-α2a in an amount of about 180 μg and Actimmune® IFN-γ in an amount of 100 μg.

In another particular embodiment, a subject admixture comprises effective amounts of IFN-α and IFN-γ, wherein the admixture comprises PEGASYS® PEGylated IFN-α2a in an amount of about 180 μg and Actimmune® IFN-γ in an amount of 150 μg.

In another particular embodiment, a subject admixture comprises effective amounts of IFN-α and IFN-γ, wherein the admixture comprises PEGASYS® PEGylated IFN-α2a in an amount of about 180 μg and Actimmune® IFN-γ in an amount of 200 μg.

In another particular embodiment, a subject admixture comprises effective amounts of IFN-α and IFN-γ, wherein the admixture comprises INFERGEN® consensus IFN-α in an amount of from about 1 μg to about 30 μg and IFN-γ in an amount of from about 10 μg to about 300 μg.

In another particular embodiment, a subject admixture comprises effective amounts of IFN-α and IFN-γ, wherein the admixture comprises INFERGEN® consensus IFN-α in an amount of 9 μg and IFN-γ in an amount of about 50 μg.

In another particular embodiment, a subject admixture comprises effective amounts of IFN-α and IFN-γ, wherein the admixture comprises INFERGEN® consensus IFN-α in an amount of 9 μg and IFN-γ in an amount of about 100 μg.

In another particular embodiment, a subject admixture comprises effective amounts of IFN-α and IFN-γ, wherein the admixture comprises INFERGEN® consensus IFN-γ in an amount of 9 μg and IFN-γ in an amount of about 150 μg.

In another particular embodiment, a subject admixture comprises effective amounts of IFN-α and IFN-γ, wherein the admixture comprises INFERGEN® consensus IFN-α in an amount of about 9 μg and Actimmune® IFN-γ in an amount of about 200 μg.

In another particular embodiment, a subject admixture comprises effective amounts of IFN-α and IFN-γ, wherein the admixture comprises INFERGEN® consensus IFN-α in an amount of 15 μg and IFN-γ in an amount of about 50 μg.

In another particular embodiment, a subject admixture comprises effective amounts of IFN-α and IFN-γ, wherein the admixture comprises INFERGEN® consensus IFN-α in an amount of 15 μg and IFN-γ in an amount of about 100 μg.

In another particular embodiment, a subject admixture comprises effective amounts of IFN-α and IFN-γ, wherein the admixture comprises INFERGEN® consensus IFN-α in an amount of 15 μg and IFN-γ in an amount of about 150 μg.

In another particular embodiment, a subject admixture comprises effective amounts of IFN-α and IFN-γ, wherein the admixture comprises INFERGEN® consensus IFN-α in an amount of about 15 μg and Actimmune® IFN-γ in an amount of about 200 μg.

In another particular embodiment, a subject admixture comprises effective amounts of IFN-α and IFN-γ, wherein the admixture comprises IFN-α 2a, 2b or 2c in an amount of from about 1 MU to about 20 MU and IFN-γ in an amount of from about 50 μg to about 300 μg.

In another particular embodiment, a subject admixture comprises effective amounts of IFN-α and IFN-γ, wherein the admixture comprises IFN-α 2a, 2b or 2c in an amount of from about 3 MU to about 10 MU and Actimmune® IFN-γ in an amount of about 50 μg.

In another particular embodiment, a subject admixture comprises effective amounts of IFN-α and IFN-γ, wherein the admixture comprises IFN-α 2a, 2b or 2c in an amount of from about 3 MU to about 10 MU and Actimmune® IFN-γ in an amount of about 100 μg.

In another particular embodiment, a subject admixture comprises effective amounts of IFN-α and IFN-γ, wherein the admixture comprises IFN-α 2a, 2b or 2c in an amount of from about 3 MU to about 10 MU and Actimmune® IFN-γ in an amount of about 150 μg.

In another particular embodiment, a subject admixture comprises effective amounts of IFN-α and IFN-γ, wherein the admixture comprises IFN-α 2a, 2b or 2c in an amount of from about 3 MU to about 10 MU and Actimmune® IFN-γ in an amount of about 200 μg.

Kits, Containers, Devices, and Systems

The present invention provides a container comprising a subject admixture. The invention further provides a kit comprising a unit dosage form of a subject admixture in a container, and a label that provides instructions for use of the kit. The invention further provides a system for delivering a subject IFN-α/IFN-γ admixture to an individual in need thereof.

The invention further provides a drug delivery device comprising (e.g., pre-loaded with) a reservoir containing an admixture that comprises a single dose of each of IFN-α and IFN-γ. In some embodiments, the present invention provides a pre-filled syringe comprising a subject admixture.

The present invention provides a subject admixture that is contained in a single reservoir, for use in a drug delivery device. In some aspects, the present invention provides a drug reservoir or other container containing a subject admixture, where an IFN-α polypeptide and an IFN-γ polypeptide are present in the admixture in an amount suitable for one dose each. The reservoir can be provided in any of a variety of forms, including, but not limited to, a cartridge, a syringe, a reservoir of a continuous delivery device, and the like.

In some embodiments, where the IFN-α/IFN-γ combination pharmaceutical composition is prepared by admixing separate IFN-α and IFN-γ aqueous solutions, the admixing takes place in a drug delivery device (e.g., a syringe) that includes two chambers, or barrels. The instant invention thus provides a syringe comprising a) a first barrel pre-filled with a first pharmaceutical composition comprising an IFN-α in a sterile aqueous solution; and b) a second barrel pre-filled with a second pharmaceutical composition comprising an IFN-γ in a sterile aqueous solution; and the admixing is effected by simultaneously introducing the contents of the first barrel and the contents of the second barrel into a common channel terminating in an aperture, e.g., a hypodermic needle or in-dwelling catheter, through which the admixture is introduced into the body of the patient.

In some embodiments, the present invention provides a container or a drug delivery device comprising (a) a first chamber pre-filled with a pharmaceutical composition comprising an IFN-α in a sterile aqueous solution; and (b) a second chamber pre-filled with a sterile lyophilized composition comprising an IFN-γ; wherein the contents of the first chamber are introduced into the second chamber prior to use, such that the lyophilized IFN-γ is solubilized or reconstituted in the IFN-α-containing sterile aqueous solution. In some of these embodiments, the IFN-α is INFERGEN® consensus IFN-α. In some of these embodiments, the IFN-γ is Actimmune® IFN-γ1b.

In some embodiments, the present invention provides a container or a drug delivery device comprising (a) a first chamber pre-filled with a pharmaceutical composition comprising an IFN-γ in a sterile aqueous solution; and (b) a second chamber pre-filled with a sterile lyophilized composition comprising an IFN-α; wherein the contents of the first chamber are introduced into the second chamber prior to use, such that the lyophilized IFN-α is solubilized or reconstituted in the IFN-γ-containing sterile aqueous solution. In some of these embodiments, the IFN-α is INFERGEN® consensus IFN-α. In some of these embodiments, the IFN-γ is Actimmune® IFN-γ1b.

In many embodiments, the device dispenses drug by moving the contents of the first chamber into the second chamber, thereby solubilizing the lyophilized IFN-α polypeptide and forming an admixture of the first and second pharmaceutical compositions in aqueous solution. In some embodiments, the first chamber comprises an outlet, from which the aqueous solution present in the first chamber is moved or extruded. In some embodiments, the second chamber comprises an inlet through which the aqueous solution enters from the first chamber. In some embodiments, the first chamber outlet and the second chamber inlet are directly connected. In other embodiments, the first chamber outlet and the second chamber inlet are connected by a channel.

In other embodiments, the first chamber and the second chamber are configured such that by application of pressure, or by removing a barrier between the first and second chambers, the first and second chambers are fused, and their contents mixed. In these embodiments, the first and second chambers are fused to form a combination chamber comprising the IFN-γ/IFN-α admixture. In some of these embodiments, the combination chamber comprises a channel connecting the combination chamber chamber to an aperture, and wherein the device is capable of moving the admixture through the channel and then expelling the admixture through the aperture.

The chambers can be any of a variety of shapes, including, e.g., cylindrical, or irregular in shape. The chambers can be composed of any of a variety of materials, including, but not limited to, a polymer (e.g., a plastic) such as a polyimide, a polyether imide, a polyether, a polyester, a polyethylene, a polycarbonate, etc.; a metal such as titanium, a metal alloy, etc.; a silicon dioxide material (e.g., glass); and the like. The material can be flexible or stiff. The chambers can be configured in any of a variety of ways relative to one another. For example, the two chambers can be in a side-by-side configuration, or in a tandem array.

The contents of the first chamber (containing the aqueous solution) can be moved into the second chamber (containing the lyophilized material) upon application of a pressure. For example, the contents of the first chamber can be moved into the second chamber by applying a pressure to a plunger that is positioned within the first chamber, at a position away from the first chamber outlet and in many embodiments at an end of the first chamber that is opposite from the first chamber outlet.

In many embodiments, the device further comprises a channel connecting the second chamber to an aperture, and wherein the device is capable of moving the admixture through the channel and then expelling the admixture through the aperture.

The contents of the first barrel (or chamber) and the second barrel (or chamber) are kept separate, and are admixed prior to use. For example, in some embodiments, the IFN-α composition and the IFN-γ composition are admixed upon use, or from about 1 second to about 1 month prior to use, e.g., from about 1 second to about to about 5 seconds, from about 5 seconds to about 10 seconds, from about 10 seconds to about 15 seconds, from about 15 seconds to about 30 seconds, from about 30 seconds to about 60 seconds, from about 60 seconds to about 5 minutes, from about 5 minutes to about 15 minutes, from about 15 minutes to about 30 minutes, from about 30 minutes to about 1 hour, from about 1 hour to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, from about 72 hours to about 4 days, from about 4 days to about 7 days, from about 7 days to about 2 weeks, or from about 2 weeks to about one month prior to use (e.g., prior to administration to an individual).

In some embodiments, the invention provides a system for delivery of an IFN-α/IFN-γ admixture to an individual in need thereof. In some embodiments, the delivery system comprises a syringe comprising two pre-filled chambers, as discussed above; and a hypodermic needle or an in-dwelling catheter. In some of these embodiments, the individual IFN-α and the IFN-γ compositions are admixed, forming a combination IFN-α/IFN-γ composition, immediately prior to injecting the combination IFN-α/IFN-γ composition into the hypodermic needle or in-dwelling catheter.

In some embodiments, the invention provides a device in which IFN-α is presented in a prefilled syringe, and IFN-γ is presented in separate pre-filled syringe. The two syringes are connected by a Luer-lock mechanism on the device prior to administration. The two products are admixed to obtain a homogenous solution and injected subcutaneously.

The invention further provides the above-described drug delivery device comprising two separate reservoirs, chambers or barrels, where the first reservoir, chamber or barrel is pre-filled with a single dose of IFN-α in a sterile aqueous solution, and where the second reservoir, chamber or barrel is pre-filled with a single dose of IFN-γ in a sterile aqueous solution. Exemplary, non-limiting drug delivery devices include injections devices, such as pen injectors, needle/syringe devices, continuous delivery devices, and the like. Any of the dosage amounts, including synergistically effective amounts, described herein can be used in the admixture, in the reservoir(s), or in the drug delivery device.

In some embodiments, the IFN-α and IFN-γ drugs are provided as liquid formulations in a dual chamber device that allows for the drugs to be mixed quickly, eliminating the need for separate vials and reducing waste, such as a Vetter Lyo-Ject device. The two liquid formulations can be stored separately with a stopper divider compatible with each drug, and the mixing and administration can be achieved with an easy twist-and-push motion. Such a device reduces drug overfills, thereby reducing cost and improving convenience in dosage administration.

Suitable containers include those adapted for administration by subcutaneous injection, including a syringe (for use with a needle), an injector pen, and the like. In some embodiments, a subject admixture is administered with a pen injector (e.g., a medication delivery pen), a number of which are known in the art. Exemplary devices which can be adapted for use herein are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable. Also suitable for use is an Intraject® needle-free injection system (Aradigm Corp.).

Interferon-Alpha

Any known IFN-α polypeptide can be used in a subject admixture. The term “interferon-alpha” as used herein refers to a family of related polypeptides that inhibit viral replication and cellular proliferation and modulate immune response. The terms “IFN-α” and “IFN-α polypeptide,” used interchangeably herein, include naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con1, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con, is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. Use of CIFN is of particular interest.

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

In some embodiments, the parent protein therapeutic is an interferon, and a known hyperglycosylated polypeptide variant comprises (1) a carbohydrate moiety covalently attached to at least one non-native glycosylation site not found in the parent interferon and/or (2) a carbohydrate moiety covalently attached to at least one native glycosylation site found but not glycosylated in the parent interferon.

In some embodiments, the known hyperglycosylated polypeptide variant is any glycosylated synthetic Type I interferon receptor polypeptide agonist described in the U.S. Provisional Patent Application for “Synthetic Type I Interferon Receptor Polypeptide Agonists” (Attorney Docket No. INTM060PRV) filed on even date herewith, the entire disclosure of which application is incorporated herein by reference.

In other embodiments, the parent polypeptide is a Type II interferon receptor polypeptide agonist. Type II interferon receptor polypeptide agonists include interferon-gamma (IFN-γ). Thus, e.g., a known hyperglycosylated polypeptide variant can be a hyperglycosylated Type II interferon receptor polypeptide agonist variant, including hyperglycosylated IFN-γ.

Suitable known hyperglycosylated polypeptide variants include hyperglycosylated forms of any parent alpha interferon polypeptide. In one aspect, a known hyperglycosylated variant of a parent alpha interferon polypeptide has an amino acid sequence that differs from the amino acid sequence of the parent polypeptide to the extent that the variant comprises one or more glycosylation sites not found in the parent polypeptide.

In another aspect, the parent polypeptide is IFN-α2a and the known hyperglycosylated polypeptide variant is an [D99N]IFN-α2a glycopeptide, where the [D99N]IFN-α2a glycopeptide is a variant of IFN-α2a having (a) an asparagine residue in place of the native aspartic acid residue at amino acid position 99 in the amino acid sequence of IFN-α2a and (b) a carbohydrate moiety covalently attached to the R-group of said asparagine residue.

In another aspect, the parent polypeptide is IFN-α2a and the known hyperglycosylated polypeptide variant is an [D99N, D105N]IFN-α2a glycopeptide, where the [D99N, D105N]IFN-α2a glycopeptide is a variant of IFN-α2a having (a) an asparagine residue in place of the native aspartic acid residue at each of amino acid positions 99 and 105 in the amino acid sequence of IFN-α2a and (b) a carbohydrate moiety covalently attached to the R-group of each of said asparagine residues.

In another aspect, the parent polypeptide is IFN-α2b and the known hyperglycosylated polypeptide variant is an [D99N]IFN-α2b glycopeptide, where the [D99N]IFN-α2b glycopeptide is a variant of IFN-α2b having (a) an asparagine residue in place of the native aspartic acid residue at amino acid position 99 in the amino acid sequence of IFN-α2b and (b) a carbohydrate moiety covalently attached to the R-group of said asparagine residue.

In another aspect, the parent polypeptide is IFN-α2b and the known hyperglycosylated polypeptide variant is an [D99N, D105N]IFN-α2b glycopeptide, where the [D99N, D105N]IFN-α2b glycopeptide is a variant of IFN-α2b having (a) an asparagine residue in place of the native aspartic acid residue at each of amino acid positions 99 and 105 in the amino acid sequence of IFN-α2b and (b) a carbohydrate moiety covalently attached to the R-group of each of said asparagine-residues.

Suitable alpha interferons further include consensus IFN-α. Consensus IFN-α (also referred to as “CIFN” and “IFN-con” and “consensus interferon”) encompasses but is not limited to the amino acid sequences designated IFN-con1, IFN-con2 and IFN-con3 which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con1 is the consensus interferon agent in the Infergen® alfacon-1 product. The Infergen® consensus interferon product is referred to herein by its brand name (Infergen®) or by its generic name (interferon alfacon-1).

Suitable known hyperglycosylated polypeptide variants include hyperglycosylated forms of any parent consensus IFN-α polypeptide. In one aspect, a known hyperglycosylated variant of a parent consensus IFN-α polypeptide has an amino acid sequence that differs from the amino acid sequence of the parent polypeptide to the extent that the variant comprises one or more glycosylation sites not found in a parent polypeptide.

In another aspect, the parent polypeptide is the interferon alfacon-1 polypeptide and the known hyperglycosylated polypeptide variant is an [D99N]interferon alfacon-1 glycopeptide, where the [D99N]interferon alfacon-1 glycopeptide is a variant of the interferon alfacon-1 polypeptide having (a) an asparagine residue substituted for the native aspartic acid residue at amino acid position 99 in the amino acid sequence of Infergen (interferon alfacon-1) and (b) a carbohydrate moiety covalently attached to the R-group of said asparagine residue.

In another aspect, the parent polypeptide is the interferon alfacon-1 polypeptide and the known hyperglycosylated polypeptide variant is an [D99N, D105N]interferon alfacon-1 glycopeptide, where the [D99N, D105N]interferon alfacon-1 glycopeptide is a variant of the interferon alfacon-1 polypeptide having (a) an asparagine residue substituted for each of the native aspartic acid residues at amino acid positions 99 and 105 in the amino acid sequence of Infergen and (b) a carbohydrate moiety covalently attached to the R-group of each of said asparagine residues.

In another aspect, the parent polypeptide is the interferon alfacon-1 polypeptide and the known hyperglycosylated polypeptide variant is an [D99N, D105N, E134N]interferon alfacon-1 glycopeptide, where the [D99N, D105N, E134N]interferon alfacon-1 glycopeptide is a variant of the interferon alfacon-1 polypeptide having (a) an asparagine residue substituted for each of the native aspartic acid, aspartic acid, and glutamic acid residues at amino acid positions 99, 105 and 134, respectively, in the amino acid sequence of Infergen and (b) a carbohydrate moiety covalently attached to the R-group of each of said asparagine residues.

In another aspect, the parent polypeptide is the interferon alfacon-1 polypeptide and the known hyperglycosylated polypeptide variant is an [D99N, E134N]interferon alfacon-1 glycopeptide, where the [D99N, E134N] interferon alfacon-1 glycopeptide is a variant of the interferon alfacon-1 polypeptide having (a) an asparagine residue substituted for each of the native aspartic acid and glutamic acid residues at amino acid positions 99 and 134, respectively, in the amino acid sequence of Infergen and (b) a carbohydrate moiety covalently attached to the R-group of each of said asparagine residues.

In another aspect, the parent polypeptide is the interferon alfacon-1 polypeptide and the known hyperglycosylated polypeptide variant is an [D105N, E134N]interferon alfacon-1 glycopeptide, where the [D105N, E134N] interferon alfacon-1 glycopeptide is a variant of the interferon alfacon-1 polypeptide having (a) an asparagine residue substituted for each of the native aspartic acid and glutamic acid residues at amino acid positions 105 S and 134, respectively, in the amino acid sequence of Infergen and (b) a carbohydrate moiety covalently attached to the R-group of each of said asparagine residues.

In another aspect, the parent polypeptide is the interferon alfacon-1 polypeptide and the known hyperglycosylated polypeptide variant is an [D99N, D105N, E134T]interferon alfacon-1 glycopeptide, where the [D99N, D105N, E134T]interferon alfacon-1 glycopeptide is a variant of the interferon alfacon-1 polypeptide having (a) an asparagine residue substituted for each of the native aspartic acid residues at amino acid positions 99 and 105 in the amino acid sequence of Infergen (b) a threonine residue substituted for the native glutamic acid residue at amino acid position 134 in the amino acid sequence of Infergen and (c) a carbohydrate moiety covalently attached to the R-group of each of said asparagine and threonine residues.

In another aspect, the parent polypeptide is the interferon alfacon-1 polypeptide and the known hyperglycosylated polypeptide variant is an [D99N, E134T]interferon alfacon-1 glycopeptide, where the [D99N, E134T]interferon alfacon-1 glycopeptide is a variant of the interferon alfacon-1 polypeptide having (a) an asparagine residue substituted for the native aspartic acid residue at amino acid position 99 in the amino acid sequence of Infergen (b) a threonine residue substituted for the native glutamic acid residue at amino acid position 134 in the amino acid sequence of Infergen and (c) a carbohydrate moiety covalently attached to the R-group of each of said asparagine and threonine residues.

In another aspect, the parent polypeptide is the interferon alfacon-1 polypeptide and the known hyperglycosylated polypeptide variant is an [D105N, E134T]interferon alfacon-1 glycopeptide, where the [D105N, E134T]interferon alfacon-1 glycopeptide is a variant of the interferon alfacon-1 polypeptide having (a) an asparagine residue substituted for the native aspartic acid residue at amino acid position 105 in the amino acid sequence of Infergen (b) a threonine residue substituted for the native glutamic acid residue at amino acid position 134 in the amino acid sequence of Infergen and (c) a carbohydrate moiety covalently attached to the R-group of each of said asparagine and threonine residues.

In another aspect, a known hyperglycosylated polypeptide variant of a parent interferon-alpha therapeutic differs from the parent interferon-alpha therapeutic to the extent that the known hyperglycosylated polypeptide variant comprises (1) a carbohydrate moiety covalently attached to a non-native glycosylation site not found in the parent interferon-alpha therapeutic and/or (2) a carbohydrate moiety covalently attached to a native glycosylation site found but not glycosylated in the parent interferon-alpha therapeutic.

PEGylated Interferon-Alpha

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group. In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10. In other embodiments, the PEGylated IFN-α is PEGylated at one or more amino acid residues from about 10 to about 28. In other embodiments, the PEGylated IFN-α is PEGylated at or near the carboxyl terminus (C-terminus) of the IFN-α polypeptide, e.g., at one or more residues from amino acids 156-166, or from amino acids 150 to 155. In other embodiments, the PEGylated IFN-α is PEGylated at one or more amino acid residues at one or more residues from amino acids 100-114.

The polyethylene glycol derivatization of amino acid residues at or near the receptor-binding and/or active site domains of the IFN-α protein can disrupt the functioning of these domains. In certain embodiments of the invention, amino acids at which PEGylation is to be avoided include amino acid residues from amino acid 30 to amino acid 40; and amino acid residues from amino acid 113 to amino acid 149.

In some embodiments, PEG is attached to IFN-α via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

Methods for making succinimidyl propionate (SPA) and succinimidyl butanoate (SBA) ester-activated PEGs are described in U.S. Pat. No. 5,672,662 (Harris, et al.) and WO 97/03106.

Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); U.S. Pat. No. 5,985,265; U.S. Pat. No. 5,672,662 (Harris, et al.) and WO 97/03106.

Pegylated IFN-α, and methods for making same, is discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N.J.), where PEGylated Roferon is known as Pegasys (Hoffman LaRoche); interferon alpha 2b (Intron, Schering-Plough, Madison, N.J.), where PEGylated Intron is known as PEG-Intron (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (Infergen®, InterMune, Inc., Brisbane, Calif.), where PEGylated Infergen is referred to as PEG-Infergen.

In many embodiments, the PEG is a monomethoxyPEG molecule that reacts with primary amine groups on the IFN-α polypeptide. Methods of modifying polypeptides with monomethoxy PEG via reductive alkylation are known in the art. See, e.g., Chamow et al. (1994) Bioconj. Chem. 5:133-140.

In one non-limiting example, PEG is linked to IFN-α via an SPA linking group. SPA esters of PEG, and methods for making same, are described in U.S. Pat. No. 5,672,662. SPA linkages provide for linkage to free amine groups on the IFN-α polypeptide.

For example, a PEG molecule is covalently attached via a linkage that comprises an amide bond between a propionyl group of the PEG moiety and the epsilon amino group of a surface-exposed lysine residue in the IFN-α polypeptide. Such a bond can be formed, e.g., by condensation of an α-methoxy, omega propanoic acid activated ester of PEG (mPEGspa).

As one non-limiting example, one monopegylated CIFN conjugate preferred for use herein has a linear PEG moiety of about 30 kD attached via a covalent linkage to the CIFN polypeptide, where the covalent linkage is an amide bond between a propionyl group of the PEG moiety and the epsilon amino group of a surface-exposed lysine residue in the CIFN polypeptide, where the surface-exposed lysine residue is chosen from lys³¹, lys⁵⁰, lys⁷¹, lys⁸⁴, lys¹²¹, lys¹²², lys¹³⁴, lys¹³⁵, and lys¹⁶⁵, and the amide bond is formed by condensation of an α-methoxy, omega propanoic acid activated ester of PEG.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pKa differences between the ε-amino groups of the lysine residues and that of the α-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized Infergen® Alfacon-1 CIFN polypeptide amino acid sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated Infergen product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., Infergen® Alfacon-1 CIFN or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The reagents selected will depend on process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of Infergen, preferably the medium will be 2-(N-morpholino)ethane sulfonic acid buffer pre-titrated to pH between 4.7 and 6.0. However, 0.1M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH-NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat chronic hepatitis C infections, liver fibrosis etc.

There may be a more reactive carboxyl group of amino acid residues elsewhere in the molecule to react with the PEG reagent and lead to monoPEGylation at that site or lead to multiple PEGylations in addition to the —COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for Infergen and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG-40kd in molecular weight and this agent could be synthesized as follows:

OH₃C—(CH₂CH₂O)n-CH₂CH₂NH₂+Glutamic Acid i.e., HOCO—CH₂CH₂CH(NH₂)—COOH is condensed with a suitable agent e.g., dicyclohexyl carbodiimide or water-soluble EDC to provide the branched chain PEG agent OH₃C—(CH₂CH₂O)ₙ—CH₂CH₂NHCOCH(NH₂)CH₂OCH₃—(CH₂CH₂O)ₙ—CH₂CH₂NHCOCH₂.

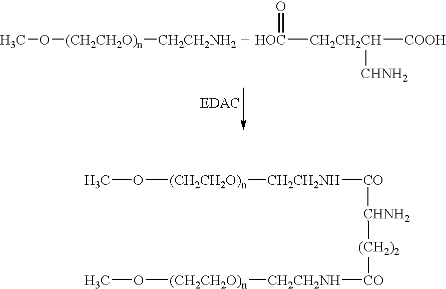

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

MonoPEG (30 kD, Linear)-ylated IFN-α

PEGylated IFN-α that is suitable for use in the present invention includes a monopegylated consensus interferon (CIFN) molecule comprised of a single CIFN polypeptide and a single polyethylene glycol (PEG) moiety, where the PEG moiety is linear and about 30 kD in molecular weight and is directly or indirectly linked through a stable covalent linkage to either the N-terminal residue in the CIFN polypeptide or a lysine residue in the CIFN polypeptide. In some embodiments, the monoPEG (30 kD, linear)-ylated IFN-α is monoPEG (30 kD, linear)-ylated INFERGEN® interferon alfacon-1.

In some embodiments, the PEG moiety is linked to either the alpha-amino group of the N-terminal residue in the CIFN polypeptide or the epsilon-amino group of a lysine residue in the CIFN polypeptide. In further embodiments, the linkage comprises an amide bond between the PEG moiety and either the alpha-amino group of the N-terminal residue or the epsilon-amino group of the lysine residue in the CIFN polypeptide. In still further embodiments, the linkage comprises an amide bond between a propionyl group of the PEG moiety and either the alpha-amino group of the N-terminal residue or the epsilon-amino group of the lysine residue in the CIFN polypeptide. In additional embodiments, the amide bond is formed by condensation of an alpha-methoxy, omega-propanoic acid activated ester of the PEG moiety and either the alpha-amino group of the N-terminal residue or the epsilon-amino group of the lysine residue in the CIFN polypeptide, thereby forming a hydrolytically stable linkage between the PEG moiety and the CIFN polypeptide.

In some embodiments, the PEG moiety is linked to the N-terminal residue in the CIFN polypeptide. In other embodiments, the PEG moiety is linked to the alpha-amino group of the N-terminal residue in the CIFN polypeptide. In further embodiments, the linkage comprises an amide bond between the PEG moiety and the alpha-amino group of the N-terminal residue in the CIFN polypeptide. In still further embodiments, the linkage comprises an amide bond between a propionyl group of the PEG moiety and the alpha-amino group of the N-terminal residue in the CIFN polypeptide. In additional embodiments, the amide bond is formed by condensation of an alpha-methoxy, omega-propanoic acid activated ester of the PEG moiety and the alpha-amino group of the N-terminal residue of the CIFN polypeptide.

In some embodiments, the PEG moiety is linked to a lysine residue in the CIFN polypeptide. In other embodiments, the PEG moiety is linked to the epsilon-amino group of a lysine residue in the CIFN polypeptide. In further embodiments, the linkage comprises an amide bond between the PEG moiety and the epsilon-amino group of the lysine group in the CIFN polypeptide. In still further embodiments, the linkage comprises an amide bond between a propionyl group of the PEG moiety and the epsilon-amino group of the lysine group in the CIFN polypeptide. In additional embodiments, the amide bond is formed by condensation of an alpha-methoxy, omega-propanoic acid activated ester of the PEG moiety and the epsilon-amino group of the lysine residue in the CIFN polypeptide.

In some embodiments, the PEG moiety is linked to a surface-exposed lysine residue in the CIFN polypeptide. In other embodiments, the PEG moiety is linked to the epsilon-amino group of a surface-exposed lysine residue in the CIFN polypeptide. In further embodiments, the linkage comprises an amide bond between the PEG moiety and the epsilon-amino group of the surface-exposed lysine residue in the CIFN polypeptide. In still further embodiments, the linkage comprises an amide bond between a propionyl group of the PEG moiety and the epsilon-amino group of the surface-exposed lysine residue in the CIFN polypeptide. In additional embodiments, the amide bond is formed by condensation of an alpha-methoxy, omega-propanoic acid activated ester of the PEG moiety and the epsilon-amino group of the surface-exposed lysine residue in the CIFN polypeptide.

In some embodiments, the PEG moiety is linked to a lysine chosen from lys³¹, lys⁵⁰, lys⁷¹, lys⁸⁴, lys¹²¹, lys¹²², lys^{134,} lys¹³⁵, and lys¹⁶⁵ of the CIFN polypeptide. In other embodiments, the PEG moiety is linked to the epsilon-amino group of a lysine chosen from lys³¹, lys⁵⁰, lys⁷¹, lys⁸⁴, lys¹²¹ lys¹²², lys¹³⁴, lys¹³⁵, and lys¹⁶⁵ of the CIFN polypeptide. In further embodiments, the linkage comprises an amide bond between the PEG moiety and the epsilon-amino group of the chosen lysine residue in the CIFN polypeptide. In still further embodiments, the linkage comprises an amide bond between a propionyl group of the PEG moiety and the epsilon-amino group of the chosen lysine residue in the CIFN polypeptide. In additional embodiments, the amide bond is formed by condensation of an alpha-methoxy, omega-propanoic acid activated ester of the PEG moiety and the epsilon-amino group of the chosen lysine residue in the CIFN polypeptide.

In some embodiments, the PEG moiety is linked to a lysine chosen from lys¹²¹, lys¹³⁴, lys¹³⁵, and lys165 of the CIFN polypeptide. In other embodiments, the PEG moiety is linked to the epsilon-amino group of a lysine chosen from lys¹²¹, lys¹³⁴, lys¹³⁵, and lys¹⁶⁵ of the CIFN polypeptide. In further embodiments, the linkage comprises an amide bond between the PEG moiety and the epsilon-amino group of the chosen lysine residue in the CIFN polypeptide. In still further embodiments, the linkage comprises an amide bond between a propionyl group of the PEG moiety and the epsilon-amino group of the chosen lysine residue in the CIFN polypeptide. In additional embodiments, the amide bond is formed by condensation of an alpha-methoxy, omega-propanoic acid activated ester of the PEG moiety and the epsilon-amino group of the chosen lysine residue in the CIFN polypeptide.

In connection with the above-described monopegylated CIFN molecules, the invention contemplates embodiments of each such molecule where the CIFN polypeptide is chosen from interferon alpha-con, interferon alpha-con₂, and interferon alpha-con₃, the amino acid sequences of which CIFN polypeptides are disclosed in U.S. Pat. No. 4,695,623.

Interferon-Gamma

Any known IFN-γ polypeptide is suitable for use in a subject admixture, provided the IFN-γ polypeptide is biologically active, e.g., modulates an immune response in a mammalian subject. The terms “IFN-γ” and “IFN-γ polypeptide,” used interchangeably herein, include naturally-occurring IFN-γ; non-naturally-occurring IFN-γ; derivatized IFN-γ (e.g., glycosylated IFN-γ, PEGylated IFN-γ, etc.); and the like. The nucleic acid sequences encoding IFN-gamma polypeptides may be accessed from public databases, e.g., Genbank, journal publications, and the like. While various mammalian IFN-gamma polypeptides are of interest, for the treatment of human disease, generally the human protein will be used. Human IFN-gamma coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ1b (Actimmune® human interferon) is a single-chain polypeptide of 140 amino acids. It is made recombinantly in E. coli and is unglycosylated (Rinderknecht et al. 1984, J. Biol. Chem. 259:6790-6797). Recombinant IFN-gamma as discussed in U.S. Pat. No. 6,497,871 is also suitable for use herein.

The IFN-gamma to be used in the admixtures and methods of the present invention may be any of natural IFN-gamma, recombinant IFN-gamma and the derivatives thereof so far as they have an IFN-γ activity, particularly human IFN-gamma activity. Human IFN-gamma exhibits the antiviral and anti-proliferative properties characteristic of the interferons, as well as a number of other immunomodulatory activities, as is known in the art. Although IFN-gamma is based on the sequences as provided above, the production of the protein and proteolytic processing can result in processing variants thereof. The unprocessed sequence provided by Gray et al., supra, consists of 166 amino acids (aa). Although the recombinant IFN-gamma produced in E. coli was originally believed to be 146 amino acids, (commencing at amino acid 20) it was subsequently found that native human IFN-gamma is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed;

For use in a subject admixture, any of the native IFN-gamma peptides, modifications and variants thereof, or a combination of one or more peptides may be used. IFN-gamma peptides of interest include fragments, and can be variously truncated at the carboxyl terminus relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-gamma moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. IFN-gamma may be modified with one or more polyethylene glycol moieties (PEGylated). In one embodiment, the invention contemplates the use of IFN-gamma variants with one or more non-naturally occurring glycosylation and/or pegylation sites that are engineered to provide glycosyl- and/or PEG-derivatized polypeptides with reduced serum clearance, such as the IFN-gamma polypeptide variants described in International Patent Publication No. WO 01/36001 and WO 02/081507. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Any known hyperglycosylated IFN-gamma polypeptide variant that retains a desired pharmacologic activity of a parent IFN-gamma polypeptide may be used in the methods and/or compositions of the invention.

In one aspect, the parent polypeptide is the mature, native IFN-gamma polypeptide; and the hyperglycosylated polypeptide variant of the parent polypeptide is an [S99T]IFN-gamma glycopeptide, where the [S99T]IFN-gamma glycopeptide is a variant of the mature, native IFN-gamma having (a) a threonine residue substituted for the native serine residue at amino acid position 99 in the amino acid sequence of IFN-gamma and (b) a carbohydrate moiety covalently attached to the R-group of the asparagine residue at amino acid position 97 in the amino acid sequence of (a).

Since the glycosylation site formed by N97, Y98, T99 in the [S99T]IFN-gamma variant is different than the glycosylation site formed by N97, Y98, S99 in native IFN-gamma, the N97, Y98, T99 glycosylation site qualifies as a non-native glycosylation site not found in the parent polypeptide. In addition, as described in WO 02/081507, the S99T substitution in the amino acid sequence of native IFN-gamma provides for greater efficiency of glycosylation at the N97, Y98, T99 glycosylation site in the [S99T]IFN-gamma variant compared to the efficiency of glycosylation at the N97, Y98, S99 glycosylation site in native IFN-gamma. Thus, [S99T]IFN-gamma qualifies as a hyperglycosylated polypeptide variant of the parent IFN-gamma polypeptide.

In another aspect, the parent polypeptide is mature, native IFN-gamma; and the hyperglycosylated polypeptide variant of the parent polypeptide is an [E38N]IFN-gamma glycopeptide, where the [E38N]IFN-gamma glycopeptide is a variant of the mature, native IFN-gamma having (a) an asparagine residue substituted for the native glutamic acid residue at amino acid position 38 in the amino acid sequence of IFN-gamma and (b) a carbohydrate moiety covalently attached to the R-group of the asparagine residue at amino acid position 38 in the amino acid sequence of (a).

In another aspect, the parent polypeptide is mature, native IFN-gamma; and the hyperglycosylated polypeptide variant of the parent polypeptide is an [E38N, S99T]IFN-gamma glycopeptide, where the [E38N, S99T]IFN-gamma glycopeptide is a variant of the mature, native IFN-gamma having (a) asparagine and threonine residues substituted for the native glutamic acid and serine residues at amino acid positions 38 and 99 in the amino acid sequence of IFN-gamma and (b) a carbohydrate moiety covalently attached to the R-group of the asparagine residue at each of amino acid positions 38 and 97 in the amino acid sequence of (a).

In another aspect, the parent polypeptide is mature, native IFN-gamma; and the hyperglycosylated polypeptide variant of the parent polypeptide is an [E38N, S40T]IFN-gamma glycopeptide, where the [E38N, S40T]IFN-gamma glycopeptide is a variant of the mature, native IFN-gamma having (a) asparagine and threonine residues substituted for the native glutamic acid and serine residues at amino acid positions 38 and 40 in the amino acid sequence of IFN-gamma and (b) a carbohydrate moiety covalently attached to the R-group of the asparagine residue at amino acid position 38 in the amino acid sequence (a).

In another aspect, the parent polypeptide is mature, native IFN-gamma having the amino acid sequence; and the hyperglycosylated polypeptide variant of the parent polypeptide is an [E38N, S40T, S99T]IFN-gamma glycopeptide, where the [E38N, S40T, S99T]IFN-gamma glycopeptide is a variant of the mature, native IFN-gamma having (a) asparagine, threonine and threonine residues substituted for the native glutamic acid, serine and serine residues at amino acid positions 38, 40 and 99, respectively, in the amino acid sequence of IFN-gamma and (b) a carbohydrate moiety covalently attached to the R-group of the asparagine residue at amino acid position 38 in the amino acid sequence of (a), and optionally further having (c) a carbohydrate moiety covalently attached to the R-group of the asparagine residue at amino acid position 97 in the amino acid sequence of IFN-gamma.

In another aspect, a known hyperglycosylated polypeptide variant of a parent interferon-gamma therapeutic differs from the parent interferon-gamma therapeutic to the extent that the known hyperglycosylated polypeptide variant comprises (1) a carbohydrate moiety covalently attached to a non-native glycosylation site not found in the parent interferon-gamma therapeutic and/or (2) a carbohydrate moiety covalently attached to a native glycosylation site found but not glycosylated in the parent interferon-gamma therapeutic.

In another aspect, the parent protein therapeutic is interferon gamma-1b and the known hyperglycosylated polypeptide variant of the parent interferon gamma-1b therapeutic is glycosylated native (wild-type) human IFN-γ as described in WO 02/081507.

Included for use in a subject admixture are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see, for example, Friedler et al. 2000, J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Treatment Methods

A subject IFN-α/IFN-γ admixture is useful for treating various disorders, including viral infections, fibrotic disorders, and proliferative disorders. Thus, the present invention provides method of treating fibrotic disorders; methods of treating proliferative disorders; and methods of treating viral infections. The subject methods generally involve administering to an individual in need thereof an effective amount of a subject IFN-α/IFN-γ admixture, wherein the subject admixture is prepared as described above, e.g., individual IFN-α and IFN-γ compositions are admixed to yield a subject IFN-α/IFN-γ admixture prior to administration of the IFN-α/IFN-γ admixture. In some embodiments, a subject treatment method further includes administering at least one additional therapeutic agent for treating a fibrotic disorder, a viral disorder, or a proliferative disorder. In some embodiments, a subject treatment method further includes administering a side effect management agent, to treat a side effect induced by a therapeutic agent (e.g., IFN-α, IFN-γ, etc.).

Fibrotic Disorders

The present invention provides methods for treating a fibrotic disorder in an individual having a fibrotic disorder. The method generally involves administering to an individual in need thereof an effective amount of a subject IFN-α/IFN-γ admixture, wherein the subject IFN-α/IFN-γ admixture is prepared as described above. The methods provide for treatment of fibrotic diseases, including those affecting the lung such as idiopathic pulmonary fibrosis, pulmonary fibrosis from a known etiology, liver fibrosis or cirrhosis, cardiac fibrosis, and renal fibrosis. The etiology may be due to any acute or chronic insult including toxic, metabolic, genetic and infectious agents.

Fibrosis is generally characterized by the pathologic or excessive accumulation of collagenous connective tissue. Fibrotic disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy), and the like), and the like.

In some embodiments, an effective amount of a subject IFN-α/IFN-γ admixture is an amount that, when administered to an individual having a fibrotic disorder, is effective to reduce fibrosis or reduce the rate of progression of fibrosis by at least about 10%, at least about 15%, at least about 20%, at least about 25%, at least about 30%, at least about 35%, at least about 40%, at least about 45%, or at least about 50%, or more, compared with the degree of fibrosis in the individual prior to treatment or compared to the rate of progression of fibrosis that would have been experienced by the patient in the absence of treatment.

In some embodiments, an effective amount of a subject IFN-α/IFN-γ admixture is an amount that, when administered to an individual having a fibrotic disorder, is effective to increase, or to reduce the rate of deterioration of, at least one function of the organ affected by fibrosis (e.g., lung, liver, kidney, etc.) by at least about 10%, at least about 15%, at least about 20%, at least about 25%, at least about 30%, at least about 35%, at least about 40%, at least about 45%, or at least about 50%, or more, compared to the basal level of organ function in the individual prior to treatment or compared to the rate of deterioration in organ function that would have been experienced by the individual in the absence of treatment.

Methods of measuring the extent of fibrosis in a given organ, and methods of measuring the function of any given organ, are well known in the art.

Idiopathic Pulmonary Fibrosis

The present invention provides methods of treating idiopathic pulmonary fibrosis (IPF). The methods generally involve administering to an individual having IPF an effective amount of a subject IFN-α/IFN-γ admixture.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

In some embodiments, an “effective amount” of a subject IFN-α/IFN-γ admixture is a dosage that is effective to decrease disease progression by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or more, compared with a placebo control or an untreated control.

Disease progression is the occurrence of one or more of the following: (1) a decrease in predicted FVC of 10% or more; (2) an increase in A-a gradient of 5 mm Hg or more; (3) a decrease of 15% of more in single breath DLc,. Whether disease progression has occurred is determined by measuring one or more of these parameters on two consecutive occasions 4 to 14 weeks apart, and comparing the value to baseline.

Thus, e.g., where an untreated or placebo-treated individual exhibits a 50% decrease in FVC over a period of time, an individual administered with an effective amount of a subject IFN-α/IFN-γ admixture exhibits a decrease in FVC of 45%, about 42%, about 40%, about 37%, about 35%, about 32%, about 30%, or less, over the same time period.

In some embodiments, an “effective amount” of a subject IFN-α/IFN-γ admixture is a dosage that is effective to increase progression-free survival time, e.g., the time from baseline (e.g., a time point from 1 day to 28 days before beginning of treatment) to death or disease progression is increased by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared a placebo-treated or an untreated control individual. Thus, e.g., in some embodiments an effective amount of a subject IFN-α/IFN-γ admixture is a dosage that is effective to increase the progression-free survival time by at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 12 months, at least about 18 months, at least about 2 years, at least about 3 years, or longer, compared to a placebo-treated or untreated control.

In some embodiments, an effective amount of a subject IFN-α/IFN-γ admixture is a dosage that is effective to increase at least one parameter of lung function, e.g., an effective amount of a subject IFN-α/IFN-γ admixture increases at least one parameter of lung function by at least about 10%, at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to an untreated individual or a placebo-treated control individual. In some of these embodiments, a determination of whether a parameter of lung function is increased is made by comparing the baseline value with the value at any time point after the beginning of treatment, e.g., 48 weeks after the beginning of treatment, or between two time points, e.g., about 4 to about 14 weeks apart, after the beginning of treatment.

In some embodiments, an effective amount of a subject IFN-α/IFN-γ admixture is a dosage that is effective to increase the FVC by at least about 10% at least about 20%, at least about 25%, at least about 30%, at least about 40%, at least about 50%, at least about 60%, at least about 70%, at least about 80%, at least about 90%, at least about 2-fold, at least about 3-fold, at least about 4-fold, at least about 5-fold, or more compared to baseline on two consecutive occasions 4 to 14 weeks apart.

In some of these embodiments, an effective amount of a subject IFN-α/IFN-γ admixture is a dosage that results in a decrease in alveolar:arterial (A-a) gradient of at least about 5 mm Hg, at least about 7 mm Hg, at least about 10 mm Hg, at least about 12 mm Hg, at least about 15 mm Hg, or more, compared to baseline.

In some of these embodiments, an effective amount of a subject IFN-α/IFN-γ admixture is a dosage that increases the single breath DL_{co} by at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, or more, compared to baseline. DL_{co} is the lung difflusing capacity for carbon monoxide, and is expressed as mL CO/mm Hg/second.

Parameters of lung function include, but are not limited to, forced vital capacity (FVC); forced expiratory volume (FEV₁); total lung capacity; partial pressure of arterial oxygen at rest; partial pressure of arterial oxygen at maximal exertion. Lung function can be measured using any known method, including, but not limited to spirometry.

Liver Fibrosis

The present invention provides methods of treating liver fibrosis, including reducing clinical liver fibrosis, reducing the likelihood that liver fibrosis will occur, and reducing a parameter associated with liver fibrosis. The methods generally involve administering an effective amount of a subject IFN-α/IFN-γ admixture to an individual in need thereof. Of particular interest in many embodiments is treatment of humans.

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver.

The present methods generally involve administering a therapeutically effective amount of a subject IFN-α/IFN-γ admixture. In some embodiments, an “effective amount” of a subject IFN-α/IFN-γ admixture is an amount that is effective in reducing liver fibrosis or reduce the rate of progression of liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

The invention also provides a method for treatment of liver fibrosis in an individual comprising administering to the individual an amount of a subject IFN-α/IFN-γ admixture that is effective for prophylaxis or therapy of liver fibrosis in the individual, e.g., increasing the probability of survival, reducing the risk of death, ameliorating the disease burden or slowing the progression of disease in the individual.

Whether treatment with a subject IFN-α/IFN-γ admixture is effective in reducing liver fibrosis is determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a subject IFN-α/IFN-γ admixture is an amount that effects a change of one unit or more in the fibrosis stage based on pre- and post-therapy liver biopsies. In particular embodiments, a therapeutically effective amount of a subject IFN-α/IFN-γ admixture reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a subject IFN-α/IFN-γ admixture. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

In another embodiment, an effective amount of a subject IFN-α/IFN-γ admixture is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or in a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

In another embodiment, a therapeutically effective amount of a subject IFN-α/IFN-γ admixture is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with a subject IFN-α/IFN-γ admixture. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequelae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

In another embodiment, a therapeutically effective amount of a subject IFN-α/IFN-γ admixture is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or in a placebo-treated individual.

Whether therapy with a subject IFN-α/IFN-γ admixture is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective amount of a subject IFN-α/IFN-γ admixture. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another embodiment, a therapeutically effective amount of a subject IFN-α/IFN-γ admixture is an amount that is effective to increase liver function by at least about 10%, at least about 20%, at least about 30%, at least about 40%, at least about 50%, at least about 60%, at least about 70%, at least about 80%, or more. For example, a therapeutically effective amount of a subject IFN-α/IFN-γ admixture is an amount that is effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a subject IFN-α/IFN-γ admixture is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

Renal Fibrosis

The present invention provides methods of treating renal fibrosis. The methods generally involve administering to an individual having renal fibrosis, or at risk of having renal fibrosis, an effective amount of a subject IFN-α/IFN-γ admixture. In some embodiments, an “effective amount” of a subject IFN-α/IFN-γ admixture that is effective in reducing renal fibrosis; and/or that is effective in reducing the likelihood that an individual will develop renal fibrosis; and/or that is effective in reducing a parameter associated with renal fibrosis; and/or that is effective in reducing a disorder associated with fibrosis of the kidney.

In one embodiment, an effective amount of a subject IFN-α/IFN-γ admixture is an amount that is sufficient to reduce renal fibrosis, or reduce the rate of progression of renal fibrosis, by at least about 10%, at least about 15%, at least about 20%, at least about 25%, at least about 30%, at least about 35%, at least about 40%, at least about 45%, at least about 50%, compared to the degree of renal fibrosis in the individual prior to treatment, or compared to the rate of progression of renal fibrosis that would have been experienced by the patient in the absence of treatment.

Whether fibrosis is reduced in the kidney is determined using any known method. For example, histochemical analysis of kidney biopsy samples for the extent of extracellular matrix (ECM) deposition and/or fibrosis is performed. Other methods are known in the art. See, e.g., Masseroli et al. (1998) Lab. Invest. 78:511-522; U.S. Pat. No. 6,214,542.

In some embodiments, an effective amount of a subject IFN-α/IFN-γ admixture is an amount that is effective to increase kidney function by at least about 10%, at least about 15%, at least about 20%, at least about 25%, at least about 30%, at least about 35%, at least about 40%, at least about 45%, at least about 50%, compared to the basal level of kidney function in the individual prior to treatment.

In some embodiments, an effective amount of a subject IFN-α/IFN-γ admixture is an amount that is effective to slow the decline in kidney function by at least about 10%, at least about 15%, at least about 20%, at least about 25%, at least about 30%, at least about 35%, at least about 40%, at least about 45%, at least about 50%, compared to the decline in kidney function that would occur in the absence of treatment.

Kidney function can be measured using any known assay, including, but not limited to, plasma creatinine level (where normal levels are generally in a range of from about 0.6 to about 1.2 mg/dL); creatinine clearance (where the normal range for creatinine clearance is from about 97 to about 137 mL/minute in men, and from about 88 to about 128 mL/minute in women); the glomerular filtration rate (either calculated or obtained from inulin clearance or other methods), blood urea nitrogen (where the normal range is from about 7 to about 20 mg/dL); and urine protein levels.

Cancer

The present invention provides a method of treating a proliferative disorder (e.g., cancer), the method generally involving administering to an individual in need thereof an effective amount of a subject admixture, wherein the subject IFN-α/IFN-γ admixture is prepared as described above.

The methods are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, an “effective amount” of a subject IFN-α/IFN-γ admixture is an amount that is sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be a genetically identical animal not treated with the subject IFN-α/IFN-γ admixture. In non-experimental systems, a suitable control may be the tumor load present before administering the subject IFN-α/IFN-γ admixture. Other suitable controls may be a placebo control.

Whether growth of a tumor is inhibited can be determined using any known method, including, but not limited to, a proliferation assay wherein the number of cells in an in vitro cell culture is measured after a period of time, where the cells are-cultured in the presence or the absence of the composition; a ³H-thymidine uptake assay; and the like.

The methods are useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

Viral Infections

The present invention provides methods of treating a virus infection, and methods of reducing viral load, or reducing the time to viral clearance, or reducing morbidity or mortality in the clinical outcomes, in patients suffering from a virus infection. The present invention further provides methods of reducing the risk that an individual will develop a pathological viral infection that has clinical sequelae. The methods generally involve administering a therapeutically effective amount of a subject IFN-α/IFN-γ admixture for the treatment of a virus infection, wherein the subject IFN-α/IFN-γ admixture is prepared as described above.

In some embodiments, a subject treatment method is prophylactic. Where a subject treatment method is prophylactic, the methods reduce the risk that an individual will develop pathological infection with a virus. An effective amount of a subject IFN-α/IFN-γ admixture is an amount that reduces the risk or reducing the probability that an individual will develop a pathological infection with a virus. For example, an effective amount reduces the risk that an individual will develop a pathological infection by at least about 10%, at least about 20%, at least about 25%, at least about 30%, at least about 35%, at least about 40%, at least about 50%, at least about 60%, at least about 70%, at least about 80%, at least about 90%, or more, compared to the risk of developing a pathological infection with the virus in the absence of treatment with a subject IFN-α/IFN-γ admixture.

In some embodiments, an effective amount of a subject IFN-α/IFN-γ admixture is an amount that reduces viral load by at least about 10%, at least about 20%, at least about 25%, at least about 30%, at least about 35%, at least about 40%, at least about 50%, at least about 60%, at least about 70%, at least about 80%, at least about 90%, or more, compared to the viral load in the absence of treatment with the subject IFN-α/IFN-γ admixture.

In some embodiments, an effective amount of a subject IFN-α/IFN-γ admixture is an amount that that reduces the time to viral clearance, by at least about 10%, at least about 20%, at least about 25%, at least about 30%, at least about 35%, at least about 40%, at least about 50%, at least about 60%, at least about 70%, at least about 80%, at least about 90%, or more, compared to the time to viral clearance in the absence of treatment.

In some embodiments, an effective amount of a subject IFN-α/IFN-γ admixture is an amount that reduces morbidity or mortality due to a virus infection by at least about 10%, at least about 20%, at least about 25%, at least about 30%, at least about 35%, at least about 40%, at least about 50%, at least about 60%, at least about 70%, at least about 80%, at least about 90%, or more, compared to the morbidity or mortality in the absence of treatment.

Whether a subject treatment method is effective in reducing the risk of a pathological virus infection, reducing viral load, reducing time to viral clearance, or reducing morbidity or mortality due to a virus infection is readily determined by those skilled in the art. Viral load is readily measured by measuring the titer or level of virus in serum. The number of virus in the serum can be determined using any known assay, including, e.g., a quantitative polymerase chain reaction assay using oligonucleotide primers specific for the virus being assayed. Whether morbidity is reduced can be determined by measuring any symptom associated with a virus infection, including, e.g., fever, respiratory symptoms (e.g., cough, ease or difficulty of breathing, and the like).

In some embodiments, the present invention provides a method of reducing viral load, and/or reducing the time to viral clearance, and/or reducing morbidity or mortality in an individual who has been exposed to a virus (e.g., an individual who has come into contact with an individual infected with a virus), the method involving administering an effective amount of subject IFN-α/IFN-γ admixture. In these embodiments, therapy is begun from about 1 hour to about 14 days following exposure, e.g., from about 1 hour to about 24 hours, from about 24 hours to about 48 hours, from about 48 hours to about 3 days, from about 3 days to about 4 days, from about 4 days to about 7 days, from about 7 days to about 10 days, or from about 10 days to about 14 days following exposure to the virus.

In some embodiments, the present invention provides a method of reducing the risk that an individual who has been exposed to a virus (e.g., an individual who has come into contact with an individual infected with a virus) will develop a pathological virus infection with clinical sequelae, the method involving administering an effective amount of a subject IFN-α/IFN-γ admixture. In these embodiments, therapy is begun from about 1 hour to about 35 days following exposure, e.g., from about 1 hour to about 24 hours, from about 24 hours to about 48 hours, from about 48 hours to about 3 days, from about 3 days to about 4 days, from about 4 days to about 7 days, from about 7 days to about 10 days, from about 10 days to about 14 days, from about 14 days to about 21 days, or from about 21 days to about 35 days following exposure to the virus.

In some embodiments, the present invention provides methods of reducing viral load, and/or reducing the time to viral clearance, and/or reducing morbidity or mortality in an individual who may or may not have been infected with a virus, and who has been exposed to a virus. In some of these embodiments, the methods involve administering an effective amount of a subject IFN-α/IFN-γ admixture within 24 hours of exposure to the virus.

In some embodiments, the present invention provides methods of reducing viral load, and/or reducing the time to viral clearance, arid/or reducing morbidity or mortality in an individual who has not been infected with a virus, and who has been exposed to a virus. In some of these embodiments, the methods involve administering an effective amount of a subject IFN-α/IFN-γ admixture within 48 hours of exposure to the virus. In other embodiments, the methods involve administering a subject IFN-α/IFN-γ admixture more than 48 hours after exposure to the virus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the virus.

In some embodiments, the present invention provides a method of reducing the risk that an individual who has been exposed to a virus will develop a pathological virus infection with clinical sequelae. In some of these embodiments, the methods involve administering an effective amount of a subject IFN-α/IFN-γ admixture within 24 hours of exposure to the virus.

In some embodiments, the present invention provides a method of reducing the risk that an individual who has been exposed to a virus (e.g., an individual who has come into contact with an individual infected with a virus) will develop a pathological viral infection with clinical sequelae. In some of these embodiments, the methods involve administering an effective amount of a subject IFN-α/IFN-γ admixture within 48 hours of exposure to the virus.

Hepatitis Virus Infection

The present invention provides methods of treating a hepatitis virus infection. In particular embodiments, the present invention provides methods of treating a hepatitis C virus (HCV) infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection. The methods generally involve administering to the individual an effective amount of a subject IFN-α/IFN-γ admixture.

In many embodiments, a subject treatment method is effective to decrease viral load in the individual, and to achieve a sustained viral response. Optionally, the subject method further provides administering to the individual an effective amount of a nucleoside analog, such as ribavirin, levovirin, isatoribine and viramidine. Of particular interest in many embodiments is treatment of humans.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

In general, an effective amount of a subject IFN-α/IFN-γ admixture is an amount that is effective to reduce viral load to undetectable levels, e.g., to less than about 5000, less than about 1000, less than about 500, or less than about 200 genome copies/mL serum. In some embodiments, an effective amount of a subject agent is an amount that is effective to reduce viral load to less than 100 genome copies/mL serum. In many embodiments, the methods of the invention achieve a sustained viral response, e.g., the viral load is reduced to undetectable levels for a period of at least about one month, at least about two months, at least about three months, at least about four months, at least about five months, or at least about six months following cessation of treatment.

Whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a subject IFN-α/IFN-γ admixture for treatment of an HCV infection is an amount effective to reduce ALT levels to less than about 45 U/ml serum.

Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, in some embodiments, the present invention provides any of the above-described methods for the treatment of HCV infection in which the subject method is modified to treat a treatment failure patient for a duration of 48 weeks.

In other embodiments, the invention provides any of the above-described methods for treatment of an HCV infection in which the subject method is modified to treat a non-responder patient, where the patient receives a 48 week course of therapy.

In other embodiments, the invention provides any of the above-described methods for the treatment of HCV infection in which the subject method is modified to treat a relapser patient, where the patient receives a 48 week course of therapy.

In other embodiments, the invention provides any of the above-described methods for the treatment of HCV infection in which the subject method is modified to treat a naive patient infected with HCV genotype 1, where the patient receives a 48 week course of therapy.

In other embodiments, the invention provides any of the above-described methods for the treatment of HCV infection in which the subject method is modified to treat a naive patient infected with HCV genotype 4, where the patient receives a 48 week course of therapy.

In other embodiments, the invention provides any of the above-described methods for the treatment of HCV infection in which the subject method is modified to treat a naive patient infected with HCV genotype 1, where the patient has a high viral load (HVL), where “HVL” refers to an HCV viral load of greater than 2×10⁶ HCV genome copies per mL serum, and where the patient receives a 48 week course of therapy.

In one embodiment, the invention provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 24 weeks.

In another embodiment, the invention provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 24 weeks to about 48 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

In another embodiment, the invention provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

In another embodiment, the invention provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

In another embodiment, the invention provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

IFN-α/IFN-γ Admixture in Combination with IFN-αTherapy and/or IFN-γ Therapy

The methods of the invention contemplate the use of a subject IFN-α/IFN-γ admixture, in combination with separately administered IFN-α and/or separately administered IFN-γ, in the treatment of a disease in a patient. Thus, the invention contemplates any of the above-described methods for treating a disease in a patient, where the patient receives a therapeutically effective combination of at least one administration of a subject IFN-α/IFN-γ admixture and at least one separate administration of IFN-α and/or IFN-γ. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection.

In other embodiments, the invention provides any of the above-described methods for treating a disease in a patient, where the patient receives a therapeutically effective combination of an amount of a subject IFN-α/IFN-γ admixture and a separately administered amount of IFN-α. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. The separately administered IFN-α is in some embodiments administered on the same day as the IFN-α/IFN-γ admixture. In other embodiments, the separately administered IFN-γ is administered one, two, three, four, five, six, or more days before or after the day on which the IFN-α/IFN-γ admixture is administered. As one non-limiting example, the description of the foregoing regimens contemplates a dosing regimen in which the patient receives a subject IFN-α/IFN-γ admixture containing 9 μg INFERGEN® consensus IFN-α and 50 μg Actimmune® IFN-γ; and separately, the patient receives 9 μg IFN-α, e.g., 9 μg INFERGEN® consensus IFN-α. As one non-limiting example, in some embodiments, the patient receives a subject IFN-α/IFN-γ admixture containing 9 μg INFERGEN® consensus IFN-α and 50 μg Actimmune® IFN-γ, administered subcutaneously three times per week; and separately, the patient receives 9 μg INFERGEN® consensus IFN-α administered subcutaneously four times per week only on days on which the IFN-α/IFN-γ admixture is not administered.

In other embodiments, the invention provides any of the above-described methods for treating a disease in a patient, where the patient receives a therapeutically effective combination of an amount of a subject IFN-α/IFN-γ admixture and a separately administered amount of IFN-γ. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. The separately administered IFN-γ is in some embodiments administered on the same day as the IFN-α/IFN-γ admixture. In other embodiments, the separately administered IFN-γ is administered one, two, three, four, five, six, or more days before or after the day on which the IFN-α/IFN-γ admixture is administered. As one non-limiting example, the description of the foregoing regimens contemplates a dosing regimen in which the patient receives a subject IFN-α/IFN-γ admixture containing 9 μg INFERGEN® consensus IFN-α and 50 μg Actimmune® IFN-γ; and separately, the patient receives 50 μg IFN-γ, e.g., 50 μg Actimmune® IFN-γ.

In other embodiments, the invention provides any of the above-described methods for treating a disease in a patient, where the patient receives a therapeutically effective combination of (i) an amount of a subject IFN-α/IFN-γ admixture (ii) an amount of separately administered IFN-α and (iii) an amount of separately administered IFN-γ. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered one, two, three, four, five, six, or more days before or after the day on which the IFN-α/IFN-γ admixture is administered. As one non-limiting example, the description of the foregoing regimens contemplates a dosing regimen in which the patient receives a subject IFN-α/IFN-γ admixture containing 9 μg INFERGEN® consensus IFN-α and 50 μg Actimmune® IFN-γ; and separately, the patient receives 9 μg IFN-α, e.g., INFERGEN® IFN-α and 50 μg IFN-γ, e.g., 50 μg Actimmune® IFN-γ.

In other embodiments, the invention provides any of the above-described methods for treating a disease in a patient, where the patient receives a therapeutically effective combination of (i) an amount of a subject IFN-α/IFN-γ admixture administered once per week and (ii) an amount of IFN-α separately administered once per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-60 . In some of these embodiments, the IFN-γ is an unpegylated IFN-γ, such as interferon gamma-1b. The separately administered IFN-α is in some embodiments administered on the same day as the IFN-α/IFN-γ admixture. In other embodiments, the separately administered IFN-α is administered one, two, three, four, five, or six days before or after the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 150 μg IFN-α, e.g., 150 μg monoPEG (30 kD, linear)-ylated consensus IFN-α, once per week.

In another embodiment, the invention provides any of the above-described methods for treating a disease in a patient, where the patient receives a therapeutically effective combination of (i) an amount of a subject IFN-α/IFN-γ admixture administered once per week and (ii) an amount of IFN-γ separately administered once per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. The separately administered IFN-γ is in some embodiments administered on the same day as the IFN-α/IFN-γ admixture. In other embodiments, the separately administered IFN-γ is administered one, two, three, four, five, or six days before or after the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 100 μg IFN-γ, e.g., 100 μg Actimmune® IFN-γ, once per week.

In another embodiment, the invention provides any of the above-described methods for treating a disease in a patient, where the patient receives a therapeutically effective combination of (i) an amount of a subject IFN-α/IFN-γ admixture administered once per week and (ii) an amount of IFN-α separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. In some of these embodiments, the IFN-α is an unpegylated IFN-α, such as interferon alfa-2a, interferon alfa-2b, interferon alfa-2c or interferon alfacon-1. In other embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. The separately administered IFN-α is in some embodiments administered on the same day as the IFN-α/IFN-γ admixture. In other embodiments, the separately administered IFN-α is administered on different days from the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 150 μg IFN-α, e.g., 150 μg monoPEG (30 kD, linear)-ylated consensus IFN-α, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered once per week and (ii) an amount of IFN-γ separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. The separately administered IFN-γ is in some embodiments administered on the same day as the IFN-α/IFN-γ admixture. In other embodiments, the separately administered IFN-γ is administered on different days from the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 100 μg IFN-γ, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered two or more times per week and (ii) an amount of IFN-α separately administered once per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. In some of these embodiments, the IFN-α is an unpegylated IFN-α, such as interferon alfa-2a, interferon alfa-2b, interferon alfa-2c or interferon alfacon-1. In other embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. In another embodiment, the invention provides any of the above-described methods for treating a disease in a patient, where the patient receives a therapeutically effective combination of (i) an amount of a subject IFN-α/IFN-γ admixture administered once per week and (ii) an amount of IFN-α separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. In some of these embodiments, the IFN-α is an unpegylated IFN-α, such as interferon alfa-2a, interferon alfa-2b, interferon alfa-2c or interferon alfacon-1. In other embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. The separately administered IFN-α is in some embodiments administered on the same day as the one of the days on which the IFN-α/IFN-γ admixture is administered. In other embodiments, the separately administered IFN-α is administered on a different day from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 150 μg IFN-α, e.g., 150 μg monoPEG (30 kD, linear)-ylated consensus IFN-α, once per week.

In another embodiment, the invention provides any of the above-described methods for treating a disease in a patient, where the patient receives a therapeutically effective combination of (i) an amount of a subject IFN-α/IFN-γ admixture administered two or more times per week and (ii) an amount of IFN-γ separately administered once per week for the desired duration of therapy. In some embodiments, the subject IFN-α/IFN-γ admixture is presented as the combination of IFN-α and IFN-γ doses contained in any of the IFN-αL/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In another embodiment, the invention provides any of the above-described methods for treating a disease in a patient, where the patient receives a therapeutically effective combination of (i) an amount of a subject IFN-α/IFN-γ admixture administered once per week and (ii) an amount of IFN-γ separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In some of these embodiments, the IFN-γ is an unpegylated IFN-γ, such as interferon gamma-1b. The separately administered IFN-γ is in some embodiments administered on the same day as the one of the days on which the IFN-α/IFN-γ admixture is administered. In other embodiments, the separately administered IFN-γ is administered on a different day from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 100 μg IFN-γ, e.g., 100 μg Actimmune® IFN-γ, once per week.

In another embodiment, the invention provides any of the above-described methods for treating a disease in a patient, where the patient receives a therapeutically effective combination of (i) an amount of a subject IFN-α/IFN-γ admixture administered two or more times per week and (ii) an amount of IFN-α separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture and any separately administered IFN-α are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. The separately administered IFN-α is in some embodiments administered on the same days as the days on which the IFN-α/IFN-γ admixture is administered. In other embodiments, the separately administered IFN-α is administered on different days from the days on which the IFN-α/IFN-γ admixture is administered. For example, the separately administered IFN-α is in some embodiments administered on alternating days from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 150 μg IFN-α, e.g., 150 μg monoPEG (30 kD, linear)-ylated consensus IFN-α, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered two or more times per week and (ii) an amount of IFN-γ separately administered two or more times per week for the desired duration of therapy. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-γ is presented as an IFN-γ dose equal to the amount of IFN-γ in the subject IFN-α/IFN-γ admixture. In some of these embodiments, the subject IFN-α/IFN-γ admixture and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. The separately administered IFN-γ is in some embodiments administered on the same days as the days on which the IFN-α/IFN-γ admixture is administered. In other embodiments, the separately administered IFN-γ is administered on different days from the days on which the IFN-α/IFN-γ admixture is administered. For example, the separately administered IFN-γ is in some embodiments administered on alternating days from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 100 μg IFN-γ, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered once per week (ii) an amount of IFN-α separately administered two or more times per week and (iii) an amount of IFN-γ separately administered once per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on a different day(s) from the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 150 μg IFN-α, e.g., 150 μg monoPEG (30 kD, linear)-ylated consensus IFN-α, two or more times per week; and 100 μg IFN-α, e.g., 100 μg Actimmune® IFN-γ, once per week.

In another embodiment, the invention provides any of the above-described methods for treating a disease in a patient, where the patient receives a therapeutically effective combination of (i) an amount of a subject IFN-α/IFN-γ admixture administered once per week (ii) an amount of IFN-α separately administered once per week and (iii) an amount of IFN-γ separately administered two or more times per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon- 1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different day(s) from the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 150 μg IFN-α, e.g., 150 μg monoPEG (30 kD, linear)-ylated consensus IFN-α, once per week; and 100 μg IFN-α, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered once per week (ii) an amount of IFN-α separately administered two or more times per week and (iii) an amount of IFN-γ separately administered two or more times per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different days from the day on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, once per week; and separately, the patient receives 150 μg IFN-α, e.g., 150 μg monoPEG (30 kD, linear)-ylated consensus IFN-α, two or more times per week (e.g., tiw); and 100 μg IFN-α, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered two or more times per week (ii) an amount of IFN-α separately administered once per week and (iii) an amount of IFN-γ separately administered once per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different day(s) from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 150 μg IFN-α, e.g., 150 μg monoPEG (30 kD, linear)-ylated consensus IFN-α, once per week; and 100 μg IFN-α, e.g., 100 μg Actimmune® IFN-γ, once per week.

In another embodiment, the invention provides any of the above-described methods for treating a disease in a patient, where the patient receives a therapeutically effective combination of (i) an amount of a subject IFN-α/IFN-γ admixture administered two or more times per week (ii) an amount of IFN-α separately administered two or more times per week and (iii) an amount of IFN-γ separately administered once per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different day(s) from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg. Actimmune® IFN-γ, two or more times per week (e.g., tiw); and separately, the patient receives 150 μg IFN-α, e.g., 150 μg monoPEG (30 kD, linear)-ylated consensus IFN-α, two or more times per week (e.g., tiw); and 100 μg IFN-α, e.g., 100 μg Actimmune® IFN-γ, once per week.

In another embodiment, the invention provides any of the above-described methods for treating a disease in a patient, where the patient receives a therapeutically effective combination of (i) an amount of a subject IFN-α/IFN-γ admixture administered two or more times per week (ii) an amount of IFN-α separately administered once per week and (iii) an amount of IFN-γ separately administered two or more times per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different day(s) from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 150 μg IFN-α, e.g., 150 μg monoPEG (30 kD, linear)-ylated consensus IFN-α, once per week; and 100 μg IFN-α, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered two or more times per week (ii) an amount of IFN-α separately administered two or more times per week and (iii) an amount of IFN-γ separately administered two or more times per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, any separately administered IFN-α is presented as an IFN-α dose equal to the amount of IFN-α in the subject IFN-α/IFN-γ admixture, and any separately administered IFN-γ is presented as an IFN-γ dose equal to the amount of IFN-γ in the subject IFN-α/IFN-γ admixture. In some of these embodiments, the subject IFN-α/IFN-γ admixture, any separately administered IFN-α, and any separately administered IFN-γ are delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b. In some embodiments, the separately administered IFN-γ and the separately administered IFN-α are administered on the same day as the IFN-α/IFN-γ admixture. In other embodiments, the separately administered IFN-γ and/or the separately administered IFN-α is/are individually administered on different day(s) from the days on which the IFN-α/IFN-γ admixture is administered. As one non-limiting example, the description of the foregoing regimens contemplates a dosing regimen in which the patient receives a subject IFN-α/IFN-γ admixture containing 150 μg monoPEG (30 kD, linear)-ylated consensus IFN-α and 100 μg Actimmune® IFN-γ, two or more times per week (e.g., tiw); and separately, the patient receives 150 μg IFN-α, e.g., 150 μg monoPEG (30 kD, linear)-ylated consensus IFN-α, two or more times per week (e.g., tiw); and 100 μg IFN-α, e.g., 100 μg Actimmune® IFN-γ, two or more times per week (e.g., tiw).

In another embodiment, the invention provides any of the above-described methods for treating a disease in a patient, where the patient receives a therapeutically effective combination of (i) an amount of a subject IFN-α/IFN-γ admixture administered three times per week and (ii) an amount of IFN-α separately administered four times per week, for the desired treatment duration. In some embodiments, the subject IFN-α/IFN-γ admixture is presented as the combination of IFN-α and IFN-γ doses contained in any of the IFN-α/IFN-γ admixtures described above. In other embodiments, the subject IFN-α/IFN-γ admixture is presented as the combination of IFN-α and IFN-γ doses contained in any of the IFN-α/IFN-γ admixtures described above, and any separately administered IFN-α is presented as an IFN-α dose equal to the amount of IFN-α in the subject IFN-α/IFN-γ admixture. In some of these embodiments, the subject IFN-α/IFN-γ admixture, and any separately administered IFN-α are delivered to the patient by subcutaneous injection. In some of these embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b.

In another embodiment, the invention provides any of the above-described methods using combination therapy with a subject IFN-α/IFN-γ admixture and separately administered IFN-α and/or IFN-γ, where the patient receives no dose of the subject admixture on the same day as any dose of separately administered IFN-α or IFN-γ.

When practicing the above-described methods utilizing a subject IFN-α/IFN-γ admixture and separately administered IFN-α and/or IFN-γ, it will be appreciated that the aggregate dosage of IFN-α and IFN-γ received by the patient, i.e., the aggregate amount of IFN-α and IFN-γ received by the patient from administrations of the subject IFN-α/IFN-γ admixture and from separate administrations of IFN-α and/or IFN-γ, over the desired duration of therapy can remain constant despite the use of different drug configurations (i.e., IFN-α/IFN-γ admixtures, separately administered IFN-α, and separately administered IFN-γ) in the various dosing regimens. While many methods of the invention may share a common total dosage of IFN-α and IFN-γ delivered to the patient, such methods may nevertheless allocate different amounts of IFN-α and IFN-γ to the IFN-αIFN-γ admixture configuration, the separately administered IFN-αconfiguration, and the separately administered IFN-γ configuration.

IFN-α/IFN-γ) Admixture Without IFN-α Therapy/IFN-γ Therapy

In addition, the methods of the invention contemplate the use of a subject IFN-α/IFN-γ admixture, without any separately administered IFN-α or separately administered IFN-γ, in the treatment of a disease in a patient. Thus, the invention contemplates any of the above-described methods for treating a disease in a patient, where the patient receives a therapeutically effective amount of a subject IFN-α/IFN-γ admixture without any separate administration of IFN-α or IFN-γ. In some embodiments, the subject IFN-α/IFN-γ admixture is presented as the combination of IFN-α and IFN-γ doses contained in any of the IFN-α/IFN-γ admixtures described above. In some of these embodiments, the subject IFN-α/IFN-γ admixture is delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b.

In one embodiment, the invention provides any of the above-described methods of treating a disease in a patient, where the patient receives a therapeutically effective amount of a subject IFN-α/IFN-γ admixture in a single dose per week for the desired duration of treatment. In some embodiments, the subject IFN-α/IFN-γ admixture is presented as the combination of IFN-α and IFN-γ doses contained in any of the IFN-α/IFN-γ admixtures described above. In some of these embodiments, the subject IFN-α/IFN-γ admixture is delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b.

In another embodiment, the invention provides any of the above-described methods of treating a disease in a patient, where the patient receives a therapeutically effective amount of a subject IFN-α/IFN-γ admixture two or more times per week for the desired duration of treatment. In some embodiments, the subject IFN-α/IFN-γ admixture is presented as the combination of IFN-α and IFN-γ doses contained in any of the IFN-α/IFN-γ admixtures described above. In some of these embodiments, the subject IFN-α/IFN-γ admixture is delivered to the patient by subcutaneous injection. In some of these embodiments, the IFN-α is a pegylated IFN-α, such as peginterferon alfa-2a, peginterferon alfa-2b or monoPEG (30 kD, linear)-ylated consensus IFN-α. In other embodiments, the IFN-α is an unpegylated IFN-α, such as interferon alfa-2a, interferon alfa-2b, interferon alfa-2c or interferon alfacon-1. In some of these embodiments, the IFN-γ is an unpegylated IFN-γ, such as interferon gamma-1b.

In another embodiment, the invention provides any of the above-described methods of treating a disease in a patient, where the patient receives a therapeutically effective amount of a subject IFN-α/IFN-γ admixture three times per week for the desired duration of treatment. In some embodiments, the subject IFN-α/IFN-γ admixture is presented as the combination of IFN-α and IFN-γ doses contained in any of the IFN-α/IFN-γ admixtures described above. In some of these embodiments, the subject IFN-α/IFN-γ admixture is delivered to the patient by subcutaneous injection. In some of these embodiments, the IFN-α is an unpegylated IFN-α, such as interferon alfa-2a, interferon alfa-2b, interferon alfa-2c or interferon alfacon-1: In some of these embodiments, the IFN-γ is an unpegylated IFN-γ, such as interferon gamma-1b.

In another embodiment, the invention provides any of the above-described methods of treating a disease in a patient, where the patient receives a therapeutically effective amount of a subject IFN-α/IFN-γ admixture seven times per week for the desired duration of treatment. In some embodiments, the subject IFN-α/IFN-γ admixture is presented as the combination of IFN-α and IFN-γ doses contained in any of the IFN-α/IFN-γ admixtures described above. In some of these embodiments, the subject IFN-α/IFN-γ admixture is delivered to the patient by subcutaneous injection. In some of these embodiments, the IFN-α is an unpegylated IFN-α, such as interferon alfa-2a, interferon alfa-2b, interferon alfa-2c or interferon alfacon-1 . In some of these embodiments, the IFN-γ is an unpegylated IFN-γ, such as interferon gamma-1b.

Dosages, Formulations, and Routes of Administration

A therapeutic agent (e.g., a subject IFN-γ/IFN-α admixture; or other active agent discussed herein), also referred to herein as “an active agent,” is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent (e.g., a subject IFN-γ/IFN-α admixture, etc.) may be administered to the host using any convenient means capable of resulting in the desired therapeutic effect. Thus, an active agent (e.g., a subject IFN-γ/IFN-αadmixture) can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent (e.g., a subject IFN-γ/IFN-αadmixture; etc.) can be achieved in various ways, including oral, buccal, rectal, parenteral, intraperitoneal, intradermal, subcutaneous, intramuscular, transdermal, intratracheal, etc., administration. In some embodiments, two different routes of administration are used. For example, in some embodiments, in some embodiments, a subject IFN-α/IFN-γ admixture may be administered via subcutaneous injection, while another agent is administered orally.

Subcutaneous administration of a therapeutic agent (e.g., a subject IFN-γ/IFN-αadmixture; etc.) can be accomplished using standard methods and devices, e.g., needle and syringe, a dual chambered syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a therapeutic agent is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of an active agent (e.g., a subject IFN-γ/IFN-α admixture) can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred in some embodiments due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, an active agent (e.g., a subject IFN-γ/IFN-αadmixture, etc.) may be administered in the form of a pharmaceutically acceptable salt of the active agent, or an active agent may be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, a therapeutic agent is formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the active agent(s) in a composition as a solution in sterile water, normal saline or another pharmaceutically acceptable carrier.

For enteral delivery, a formulation will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

Suitable oral formulations also include an active agent formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B. V.).

Suitable oral formulations also include an active agent formulated with any of the following: a carrier such as Emisphere® (Emisphere Technologies, Inc.); TIMERx, a hydrophilic matrix combining xanthfan and locust bean gums which, in the presence of dextrose, form a strong binder gel in water (Penwest); Geminex™ (Penwest); Procisem (GlaxoSmithKline); SAVITT™ (Mistral Pharma Inc.); RingCap™ (Alza Corp.); Smartrix™ (Smartrix Technologies, Inc.); SQZgel™ (MacroMed, Inc.); Geomatrix™ (Skye Pharma, Inc.); Prosy Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Dosages

The term “it dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for the unit dosage forms of the present invention depend on the particular active agent employed and the effect to be achieved, and the pharmacodynamics associated with each compound in the host.

A subject IFN-α/IFN-γ admixture can be administered twice daily, daily, every other day, once a week, twice a week, three times a week, every other week, three times per month, or once monthly, or substantially continuously or continuously.

A subject IFN-α/IFN-γ admixture is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

Subjects Suitable for Treatment

The subject methods are suitable for treating individuals having, or susceptible to having, a variety of disorders. In many embodiments of interest, the individual is a human.

Fibrotic Disorders

The subject methods for treating fibrotic disorders are suitable for treatment of individuals diagnosed as having a fibrotic disorder. Fibrotic disorders include, but are not limited to, pulmonary fibrosis, including idiopathic pulmonary fibrosis (IPF) and pulmonary fibrosis from a known etiology, liver fibrosis, and renal fibrosis. Other exemplary fibrotic conditions include musculoskeletal fibrosis, cardiac fibrosis, post-surgical adhesions, scleroderma, glaucoma, and skin lesions such as keloids.

Cancer

Subjects suitable for treatment with a subject method for treating cancer include individuals having any type of cancer. Also suitable for treatment are individuals who have failed previous treatment for a cancer with a standard cancer chemotherapeutic agent. Also suitable for treatment are individuals who have been previously treated with a standard cancer chemotherapeutic agent, who initially responded to such treatment, and in whom the cancer subsequently reappeared. Also suitable for treatment are individuals who failed to respond to treatment with another agent for treating cancer.

HCV Infection

Individuals who are to be treated according to the methods of the invention for treating an HCV infection include individuals who have been clinically diagnosed as infected with HCV. Individuals who are infected with HCV are identified as having HCV RNA in their blood, and/or having anti-HCV antibody in their serum.

Individuals who are clinically diagnosed as infected with HCV include naive individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-αmonotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods of the present invention. In other embodiments, individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system.).

Therapy With Interferon Alphacon 1 and Interferon Gamma-1b

Interferons (IFNs) are cytokines induced as a consequence of viral and microbial infections and have pleiotropic biological effects that modulate innate and adaptive immunity. Type I and type II IFNs activate distinct signal transduction pathways involving Jak-STAT and other components, resulting in the execution of overlapping programs of gene regulation through induction of IFN-stimulated genes (ISGs).

While type I IFNs have been used clinically to treat a broad variety of viral infections, regimens containing type I IFNs constitute the standard of care for chronic hepatitis C virus infection. To help gauge the molecular processes impacting the sustained virologic response rate (SVR) following treatment with such regimens the kinetics of HCV RNA clearance in individual patients has been examined. HCV clearance in response to IFN-α-based therapy has been shown to follow a biphasic pattern, with a rapid initial phase attributed to induction of the direct antiviral effectors in infected hepatocytes by IFN-α and the possible RNA mutagenic properties and HCV RNA-dependent RNA-polymerase (RdRp)-inhibitory activities of ribavirin. The slower second phase of viral clearance has been attributed to cellular immune responses resulting in the death of cells harboring HCV. Type I IFNs are thought to potentiate the first phase of viral clearance by inducing direct antiviral components such as 2′-5′ OAS, PKR, and Mx proteins. Liver infiltration of macrophage, T-helper 1 (Th1), and natural killer (NK) cells mediated in part by the induction of IFN-γ may be responsible for the killing of infected cells in the second phase of clearance.

IFN-α induced activity during both of these phases is likely important for SVR following treatment of chronic hepatitis C patients. Type I IFN-mediated effects in the first phase of clearance are likely to be important for SVR because the down-regulation of type I IFN receptor in chronically infected patients, which would attenuate type I effects, has been correlated to treatment nonresponse. In the second phase of viral clearance, IFN-γ (type II IFN)-induced mobilization of Th1 responses has been correlated with SVR. In addition, although IFN-γ when used alone is a less potent antiviral agent in vitro than IFN-α on a per mass unit basis, it may potentiate type I IFN effects in the first phase of viral clearance since it has displayed synergistic antiviral activity with type I IFN in several systems and a HCV subgenomic replicon.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Analysis of Short-Term Stability of IFN-α/IFN-γ Admixture

IFN-α/IFN-γ Formulations

The feasibility to develop a single container configuration of interferons (alpha plus gamma) was assessed in a preliminary study. The objective of this study was to assess the physical and chemical integrity of Infergen® and Actimmune® after mixing them together in one container. This study was designed to evaluate the integrity and stability of this mixture by analyzing the mixed samples at various time points after mixing. The analyses included monitoring of turbidity at 320 nm and 600 nm; aggregation of the polypeptides by size exclusion chromatography and chemical integrity of the individual proteins by reversed phase high performance liquid chromatography (RP-HPLC). In the one-day stability study, the samples were evaluated right after mixing and after 2 hours, 4 hours, and 8 hours of incubation at room temperature.

Infergen® and Actimmune® are the preparations of consensus interferon alpha (IFN-alfacon-1) and interferon gamma-1b, respectively, that differ in their therapeutic properties. It was conceived that in the context of interferon-α and interferon-γ combination therapy for treatment of chronic hepatitis C, an ability to admix interferon-α and interferon-γ drugs could allow the administration of an admixture in place of separate administrations of interferon-α and interferon-γ, thereby reducing the number of repeat injections received by the patient. As a prototype, it was proposed to examine a combination of 15 pg of Infergen® and 100 μg of Actimmune® in 1 mL of buffered solution by mixing the contents of one vial of each of the two interferons. Succinate and phosphate buffers in which the two drugs are formulated are freely miscible. All aliquots of the drug combinations (Infergen plus Actimmune) were analyzed by size exclusion-HPLC (SEC-HPLC), RP-HPLC, UV-Vis spectroscopy (320 and 600 nm). Three assays (described below) were performed to obtain information on the physical and chemical integrity of the formulation. The presence of any precipitate or cloudiness in the container was judged by UV-Vis spectroscopy (i.e., turbidity). The aggregation state of the proteins was assessed by monitoring the monomer/oligomer compositions of the proteins (SEC-HPLC). Chemical integrity of the individual polypeptides was judged by monitoring the degradation of the components (RP-HPLC). Separately, a study to assess the biological effects of the cytokines was conducted using an antiviral assay. Since this assay was carried out in a different laboratory (MDS Pharmaceutical Services, Montreal), the formulation aliquots were treated differently and were analyzed for stability up to 24 hours at ambient temperature.

Product Formulations: Interferon alfacon-1 or Infergen was obtained as a sterile, clear, colorless, preservative-free liquid at a drug concentration of 30 μg/mL (0.5 mL fill volume). Interferon gamma-1b or Actimmune was obtained as sterile, clear, colorless solution at a drug concentration of 200 μg/mL (fill volume of 0.5 mL). Infergen was formulated in a phosphate buffered sodium chloride solution containing 5.9 mg/mL sodium chloride and 3.8 mg/mL sodium phosphate in Water for Injection at a pH of 7.0. Actimmune was formulated in 20 mg of mannitol, 0.36 mg sodium succinate, 0.05 mg polysorbate 20 and sterile Water for Injection.

Stability assessments for a day (defined as a working day of 8 hours) were carried out using three vials of Infergen and three vials of Actimmune. An aliquot of 0.5 mL of Infergen (15 μg) was withdrawn from the drug product vial using a 1 mL sterile disposable syringe with a needle and deposited into a screw-cap conical cryo vial. Approximately 0.5 mL of Actimmune (100 μg) was similarly withdrawn from the other drug product vial and deposited into the same cryo vial. The content of the cryo vial (fill volume of 1 mL) was mixed by gentle inversion of the vial ten times. Three such vials were prepared similarly and stored at ambient temperature. An aliquot of 200 μL was withdrawn from each vial at time zero (immediately after mixing), 2 hr, 4 hr and 8 hr using a sterile 1 mL syringe with neddle. The aliquots were immediately analyzed by UV-Vis and then recovered from the cell and split between two glass auto sampler vials ˜75 μL each. Following this, one vial was analyzed by SEC-HPLC and the other by RP-HPLC. The turbidity scans were recorded on an HP 8453 UV-Vis spectrophotometer. HPLC studies were conducted with an Hewlett Packard 1100 HPLC system or equivalent equipped with a binary pumping system, UV detector and an auto sampler. Either a Zorbax 300 SB-CS 4.6×150 mm column or Tosoh TSK-Gel G2000SWXL 7.8×30 cm column was used for the measurements.

Antiviral Assay

A mixture of IFN-alfacon-1 and IFN gamma-1b were mixed to provide drug concentrations of 15 μg/mL (alpha) and 100 μg/mL (gamma). This stock solution was diluted further with blank human serum to yield an interferon alpha concentrations of 0.15 μg/mL and gamma concentrations of 1.0 μg/mL as working solution. These Infergen and Actimmune drug combinations i.e., working solutions were used to prepare the standards by serial dilution. Standards were prepared fresh daily. Infergen, Actimmune and drug combination working solutions were incubated at ambient temperature for 0 hr, 2 hrs, 4 hrs, 8 hrs, and 24 hours before preparing standards.

The antiviral protection potential of a combination of IFN-alpha/IFN-gamma in a cell culture system was evaluated. The maximum standard range of quantitation was 1.0 μg/mL for IFN-gamma or 150 ng/mL for IFN-alpha and the combo IFN-alpha/IFN-gamma and was serially diluted to 5.64 pg/mL for IFN-gamma or 0.85 pg/mL for IFN-alpha and the combo IFN-alpha/IFN-gamma.

This assay employs a cell culture system in which A-549 cells are seeded into 96-well plates. After an incubation period allowing cells to grow and form a tight monolayer, IFN standards are added to the plates containing cells. After a 24 hours incubation period, the plates were washed to remove any IFN subtances. L-encephalomyocarditis (L-EMC) virus was added to the plates and incubated with the cells until cytopathic effect (CPE) is observed. Then, the reaction was stopped by the addition of a 10% formalin. A dye-uptake method using Crystal Violet solution was performed to evaluate the cell viability level after the incubation with the virus. Acetic acid was used to release the Crystal Violet solution contained in living cells. The end-result was measured by optical density at 570 nm. A dark color represents a strong antiviral protection effect.

Results: Samples of Infergen and Actimmune were used as references for the UV-Vis scans. As shown in Tables 1 and 2 below, the stability samples of the mixture of Infergen and Actimmune showed similar data dispersion as the interferons alone and showed no increases in absorbance at either wavelength at all time points.

TABLE 1
UV-Vis Analysis of Infergen Controls

| | Absorbance (320 nm) | | | Absorbance (600 nm) | | |
|---|---|---|---|---|---|---|
| sample | reading 1 | reading 2 | Average | reading 1 | reading 2 | Average |
| Infergen, Time 0 hr | 0.017383 | 0.048803 | 0.033093 | 0.012756 | 0.070708 | 0.041732 |
| Infergen, Time 2 hr | 0.040709 | 0.039518 | 0.040114 | 0.0071058 | 0.029376 | 0.018241 |
| Infergen, Time 4 hr | 0.0092783 | −0.00010939 | 0.004584 | 0.0035024 | −0.0099974 | −0.003248 |
| Infergen, Time 8 hr | 0.045300 | 0.034539 | 0.039920 | 0.027896 | 0.014994 | 0.021445 |

TABLE 2
One Day Stability of Infergen Actimmune Mixture as Analyzed by UV-Vis

| | Absorbance (320 nm) | | | | | Absorbance (600 nm) | | | | |
|---|---|---|---|---|---|---|---|---|---|---|
| sample | reading 1 | reading 2 | reading 3 | Average | std. dev. | reading 1 | reading 2 | reading 3 | Average | std. dev. |
| Mix 1, RT, time 0 hr | 0.082423 | 0.086586 | 0.077288 | 0.082099 | 0.005 | 0.022366 | 0.029802 | 0.023974 | 0.025381 | 0.004 |
| Mix 2, RT, time 0 hr | 0.076357 | 0.073568 | 0.072526 | 0.074150 | 0.002 | 0.029605 | 0.030308 | 0.031088 | 0.030334 | 0.001 |
| Mix 3, RT, time 0 hr | 0.068736 | 0.059093 | 0.056078 | 0.061302 | 0.007 | 0.018480 | 0.013278 | 0.021780 | 0.017846 | 0.004 |
| Mix 1, RT, time 2 hr | 0.092560 | 0.086369 | 0.080667 | 0.086532 | 0.006 | 0.027419 | 0.020116 | 0.020467 | 0.022667 | 0.004 |
| Mix 2, RT, time 2 hr | 0.075894 | 0.063271 | 0.050681 | 0.063282 | 0.013 | 0.051250 | 0.030181 | 0.014080 | 0.031837 | 0.019 |
| Mix 3, RT, time 2 hr | 0.065324 | 0.055395 | 0.045119 | 0.055279 | 0.010 | 0.033156 | 0.023933 | 0.054011 | 0.037033 | 0.015 |
| Mix 1, RT, time 4 hr | 0.084654 | 0.064146 | 0.044081 | 0.064294 | 0.020 | 0.044900 | 0.023582 | −0.0097647 | 0.019572 | 0.028 |
| Mix 2, RT, time 4 hr | 0.044361 | 0.029112 | 0.013756 | 0.029076 | 0.015 | 0.010466 | −0.0082397 | −0.022449 | −0.006741 | 0.017 |
| Mix 3, RT, time 4 hr | 0.01457 | 0.011817 | 0.0089564 | 0.011781 | 0.003 | −0.015921 | −0.017818 | −0.017153 | −0.016964 | 0.001 |
| Mix 1, RT, time 8 hr | 0.088333 | 0.078293 | 0.065112 | 0.077246 | 0.012 | 0.031637 | 0.026564 | 0.014203 | 0.024135 | 0.009 |
| Mix 2, RT, time 8 hr | 0.053024 | 0.054938 | 0.054643 | 0.054202 | 0.001 | 0.030066 | 0.032248 | 0.031713 | 0.031342 | 0.001 |
| Mix 3, RT, time 8 hr | 0.046886 | 0.43438 | 0.041700 | 0.044008 | 0.003 | 0.022069 | 0.018144 | 0.019783 | 0.019999 | 0.002 |

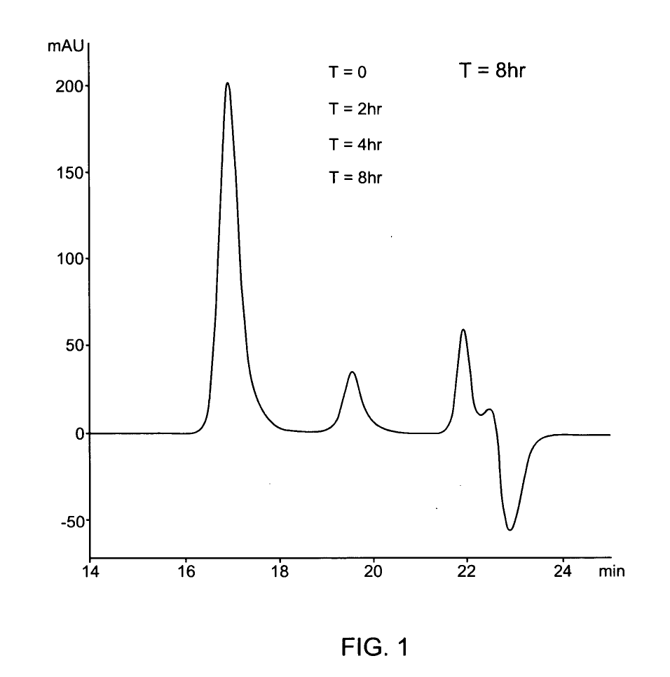

FIG. 1 shows the chromatographic overlays for all time points of the SEC-HPLC traces. No difference is observed in the chromatographic profiles between Infergen and Actimmune samples right after mixing and up to 8 hours of storage at ambient temperature. The tabulated data of peak areas and retention times for all time points and individual samples also did not indicate any change in mixture upon storage.

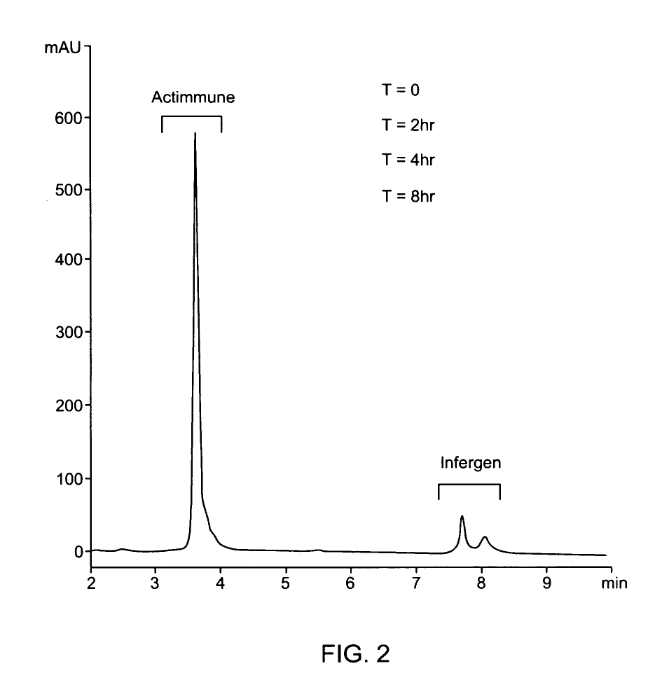

FIG. 2 shows the chromatographic overlays for all time points for the RP-HPLC traces. Again, no difference is observed in the chromatograms between the combined polypeptides immediately after mixing and storage at ambient temperature for up to 8 hours. A comparison of the peak properties of the individual curves, such as peak retention times and areas, also indicated no change in the alpha/gamma mixture upon storage.

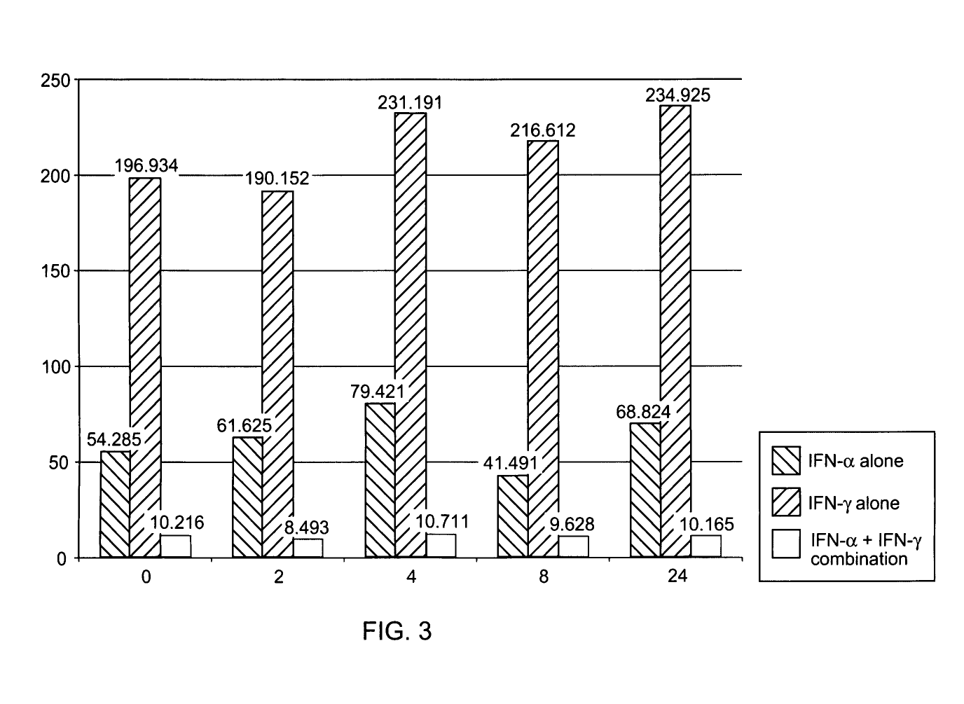

The antiviral activities of the interferons were expressed by calculating mean EC50 values. A mean EC50 value of 61.1292±14.37487 pg/mL was obtained for Infergen, 213.9628±20.00111 pg/mL for Actimmune and 9.8426±0.84631 pg/mL for alpha/gamma combination. The stability of the single container mixture was assessed by plotting the individual measurements of the antiviral activities at different time points as a histogram. As shown in FIG. 3, there were no significant changes in the EC50 values of the mixture upon storage up to 24 hours at room temperature. This indicates that the mixture retained its full biological antiviral activity upon storage in a single container. Based on all analytical methods and test results, it is believed that the drug product combination (interferons alpha plus gamma) is compatible for single container storage and administration and is suitable for clinical administration.

### Example 2

### Analysis of Synergistic Antiviral Properties of IFN-α/IFN-γ Admixture IFN-α/IFN-γ Formulations

The objective of this study was to assess whether an admixture of interferon-alpha and interferon-gamma have a synergistic antiviral effect. This study was designed to evaluate the inhibition of dengue virus in the presence of either interferon alphacon 1 or interferon-gamma1b or in the presence of both interferon alphacon 1 and interferon-gamma 1b. The analyses included determining the antiviral activity of the interferons by measuring the titer of dengue virus in cultures exposed to either interferon alphacon 1 or to interferon-gamma1b or to both interferon alphacon 1 and interferon-gamma 1b, and calculating combination index (CI) values to assess any synergistic antiviral effect. In addition, the effect of interferon alphacon 1, interferon-gamma 1b, and the combination of interferon alphacon 1 and interferon-gamma 1b on the regulation of antiviral genes in hepatocytes was determined.

Antiviral Assay

A549, Dengue Infection and Interferon Treatment

A549 cells were seeded in 6 well plates at about 5×10⁵ cells per well. The next day the media was removed and the appropriate concentration of each interferon was added in 1 ml volume according to the experimental protocol. After 24 hours an additional ml was added containing the dengue virus. The supernatant was collected after 7 days. Cell debris was removed by centrifugation and the viral supernatant was stored at −80° C. until the RNA was extracted.

RNA Extraction, RTqPCR

Viral RNA was extracted using the Qiagen QIAamp Viral RNA Kit or Roche High Pure Viral RNA kit using the manufacturer's instructions.

The reverse transcription (RT) reaction was carried out with 10 μl of RNA and the following:

| | | |
|---|---|---|
| 10X RT Buffer | 2 | μl |
| Random Hexamer | 2 | μl (0.2 μg) |
| RNAsin | 0.5 | μl (20 U) |
| DNTP | 2 | μl (250 μM) |
| MMLV-RT | 1 | μl (200 U) |
| Water | 2.5 | μl |

The RNA was heated to 80° C. for 5 minutes then iced. A master mix was made and 10 μl added per reaction. The RT was done at 42° C. for 1 hour, 65° C. for 15 minutes and hold at 4° C. in an Eppendorf Mastercycler Gradient.

The quantitative polymerase chain reaction (qPCR) primer and probe sequences were provided by Intermune. The primers were diluted to 50 μM and the probe to 10 M. Several concentrations were tested to come up with the most efficient. 5 μl of the RT reaction was used with the following:

| | | |
|---|---|---|
| 2X SuperMix | 20 | μl |
| Primer F | 1 | μl |
| Primer R | 1 | μl |
| Probe | 0.5 | μl |
| Water | 12.5 | μl |

The reactions were carried out in 96 well plates and each run included a set of standards from RT carried out with the rest of the samples with RNA standard stocks of known virus titer. The stock was 10⁶ pfu/ml and 1:10 serial dilutions were made to construct a standard curve. The PCR program consisted of a 15 minute activation cycle at 95° C., 50 cycles of 95° C. for 10 seconds and 65° C. for 45 seconds, followed by 72° C. for 3 minutes and hold at 4° C. The program was set up so the standard curve was constructed and used to calculate the virus concentrations of the unknowns. This was done on Bio-Rad iCycler iQReal-Time PCR Detection System.

The dengue virus present was determined from standard curves generated with each set of unknowns. This was organized into a Potentiation of Antiviral Activity Table as used in Oleszak, E., William StewartII, Journal of Interferon Research 5:361-371. and Fleischmann, W. et.al., Journal of Interferon Research, Vol. 4 No. 2.

Combination Index

IFNs and cell lines. Human hepatoma (Huh7) cells were treated for 24 hrs with IFN alfacon-1 (Infergen®, InterMune, Inc., Brisbane, Calif.), IFN γ-1b (Actimmune®, InterMune, Inc., Brisbane, Calif.), or both, at levels equivalent to human maximum plasma concentrations (Cₘₐₓ) obtained following FDA-approved clinical dosing schedules, which corresponded to 80 pg/mL IFN alfacon-1 and 300 pg/mL IFN γ-1b (data on file). Specific activity of IFN alfacon-1 and IFN γ-1b in antiviral assays was 1×10⁹ IU/mg and 2×10⁷ IU/mg, respectively. All experiments and calculations were performed in reference to mass amounts rather than international unit amounts. Human lung carcinoma (A549) cells were treated for 24 hrs with an empirically determined concentration range of IFN alfacon-1, IFN γ-1b, or both, as described in the text and infected with vesicular stomatitis virus (VSV) at a multiplicity of infection (M.O.I.) of 0.1. For IFN combination studies, IFN alfacon-1 to IFN γ-1b ratios of 1:60 and 1:120 were used and IFN concentration varied as described in the text. A549 cells harvested for global expression profiling were treated for 24 hrs with IFN alfacon-1, IFN γ-1b, or both, as described in the text.

Analysis of synergy. Cells were harvested and RNA extracted by standard protocols. Interferon concentrations yielding biological effects were empirically determined and concentration ranges centered on these values were used to quantitatively assess antiviral activity. The percent inhibition of viral replication was calculated for each treatment regimen by dividing VSV copy number in treated samples by the VSV copy number observed in untreated controls. Fifty and ninety percent effective concentration values (EC₅₀ and EC₉₀, respectively) were determined by median effect analysis performed on at least three experiments. Type I/type II IFN synergy in combination regimens was analyzed by examining both mutually exclusive (i.e., independent) and mutually nonexclusive (i.e., nonindependent) models for the combined action of IFN alfacon-1 and IFN γ-1b using Chou-Talalay methods in CalcuSyn (Biosoft, Cambridge, United Kingdom). As recommended for Chou-Talalay synergy analysis, the ratio of type I IFN and type II IFN used in combination studies was equal to, or 2-fold greater than, the ratio of EC₅₀s for type II and type I IFNs, respectively. The CI is plotted as the solid line versus the percent inhibition. CI values of <1, 1 and >1 indicate synergy, additive effect and antagonism respectively.

Antiviral Gene Regulation

Microarray experiments. Analysis was performed using Affymetrix (Santa Clara, Calif.) Genechip® U133 Plus 2.0 arrays (hepatocyte expression profiles) or Genechip® U133A arrays (A549 expression profiles) in accordance with manufacturer-recommended protocols. Four independent samples of each treatment regimen were used for both experiments. In one case, hepatocyte-derived cells treated with 80 pg/mL IFN alfacon-1, a microarray corresponding to one of the four replicates was not used for subsequent analysis because of poor signal quality. Data were exported from Affymetrix MAS5 software and only those probe sets called “present” in all replicates of at least one condition were used for further analysis in order to purge the data set of unreliable information. Statistical analysis was performed in Partek Pro 5.1 (Partek, St. Paul, Minn.) and clustering was performed in GeneSpring 6.1 (Silicon Genetics, Redwood City, Calif.) as described in the text. To increase statistical power in each experiment (Huh7 treated at Cₘₐₓ and A549 cells treated at EC₅₀), master lists of probe sets that detected differences in expression level were created, defined by ANOVA analysis of all treatment conditions with statistically significant differences defined by a Benjamini and Hochberg multiple test corrected p<0.05. Master lists were used as the basis for subsequent analysis as described in the text. K-means and hierarchical gene clustering was performed in GeneSpring 6.1. K-means gene clusters were created following 100 iterations using a standard correlation similarity measure. Statistical analysis was performed on log₁₀ transformed data. Genes were functionally classified using Gene Ontology designations in NetAffx (Affymetrix, Santa Clara, Calif.).

RNA quantification by real-time PCR. All real-time PCR was carried out on an ABI Prism 7900HT Sequence Detection System (Applied Biosystems, Foster City, Calif.). Expression levels of cellular RNAs were quantified in the cDNA samples used for microarray experiments and run in triplicate. Pre-existing reagents specific for human HLA-E, WARS, ISG-20, OAS2, IRF-1, STAT1, STAT2, IFIT4, MDA5, ISG20, GlP2/ISG15, C1S, PSMB8, SP110, IF144 and GAPDH were used (ABI catalogue numbers Hs00428366, Hs00188259, Hs00158122, Hs00159719, Hs00233698, Hs00234829, Hs0013115, Hs00155468, Hs00223420, Hs00158122, Hs00192713, Hs00156159, Hs00188149, Hs00270142, Hs00197427 and 4326317E, respectively). To determine cellular RNA concentrations, message levels were related to a standard curve based on linear regression of signal derived from 4-fold dilutions of cDNA from Huh7 or A549 cells treated with IFN alfacon-1 and IFN γ-1b and normalized to GAPDH expression as indicated in the text. For viral RNA quantification, RNA was reverse transcribed using antisense primer 5′-ATA ACC TAA GAA CTG GCC CAT AAC TC-3′ (SEQ ID NO:1). Real-time PCR was carried out using the same antisense primer, a 5′ sense primer (5′-CGG CCA AGG ATT GAA GTC A-3′; SEQ ID NO:2) and a Taqman probe (5′-FAM/ATT GAT TAC GAA AAA TGG AAT AAC CAC CAA AGG A/BHQ-3′; SEQ ID NO:3; where FAM=Fluorescein and 3BHQ=3′Black Hole Quencher). Primers and probe for viral RNA quantification were obtained from Integrated DNA Technologies (Coralville, Iowa) as HPLC-purified oligonucleotides. To determine viral RNA concentrations, viral RNA levels were related to a standard curve based on linear regression of signal derived from an in vitro transcribed VSV RNA fragment and normalized to raw cell count. Data analyses were performed using GeneAmp software (Applied Biosystems, Foster City, Calif.).

Results

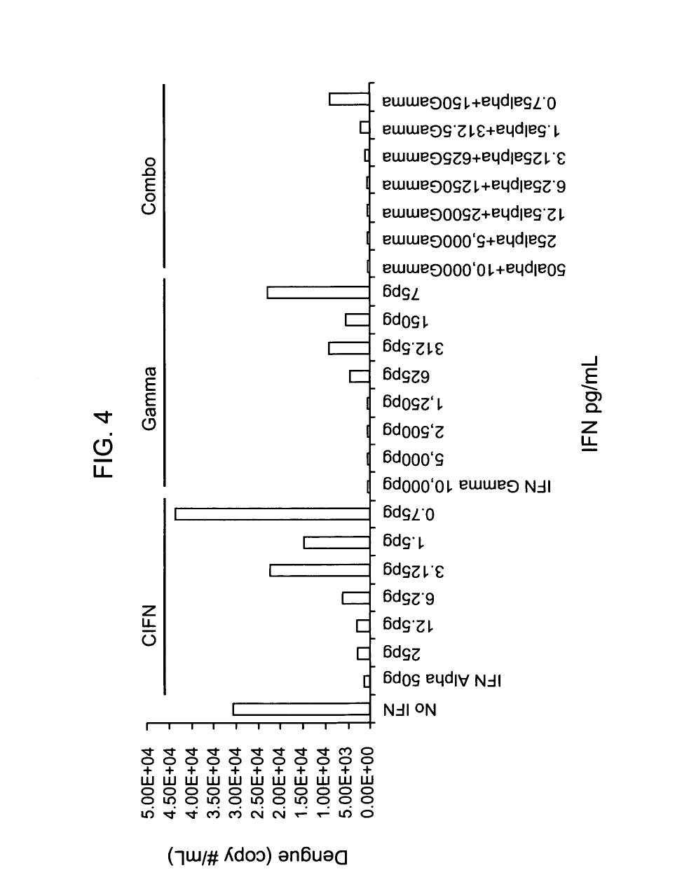

FIG. 4 shows the reduction in viral titer in the presence of each interferon alone and in the presence of the combination of both interferons. A reduction in viral titer is evident in the presence of the combination of both interferons as compared to either interferon alone.

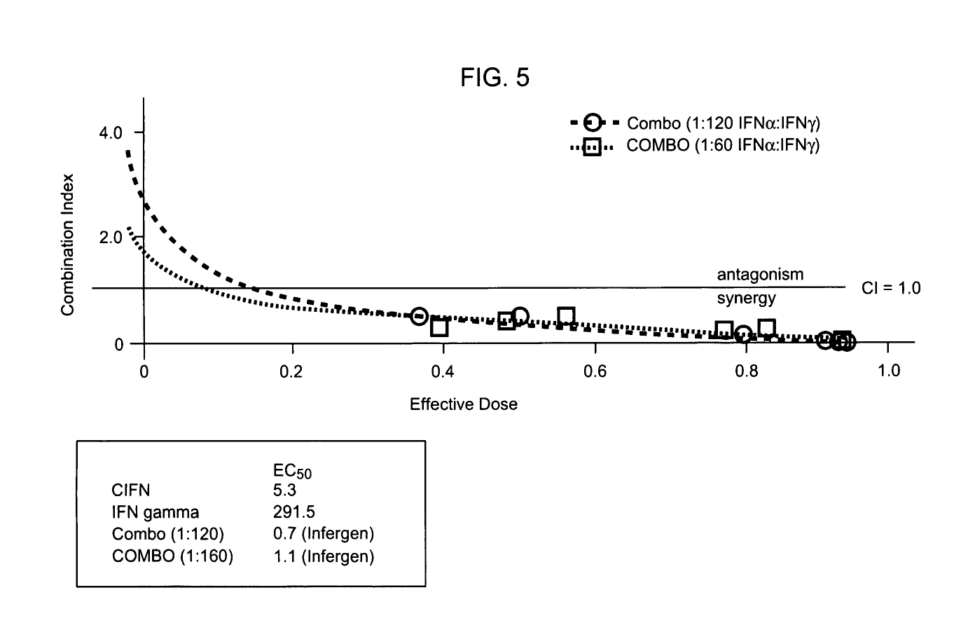

FIG. 5 shows the combination index (CI) plot of the 1:120 and 1:60 combination of interferon alphacon 1 and interferon-gamma 1b at varying effective doses, and also reports the EC₅₀ determined for each interferon alone and for the 1:120 and 1:60 combinations. Both the 1:120 and 1:60 combinations exhibit synergism at all effective doses.

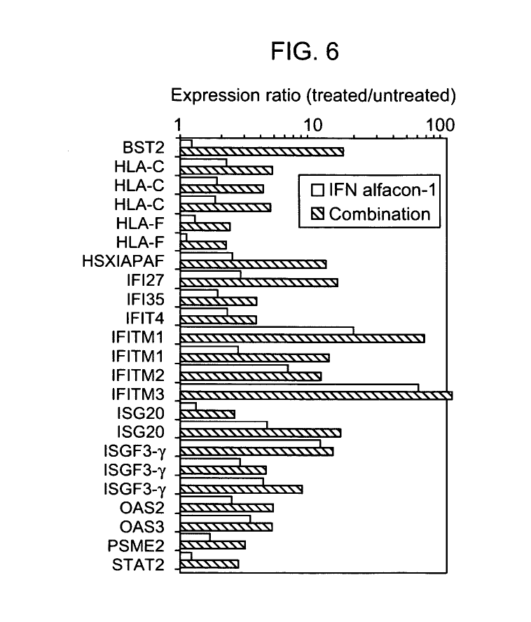

FIG. 6 shows the level of expression in a select antiviral-associated gene set in hepatocyte-derived cells following exposure to interferon alphacon 1 alone or to interferon alphacon 1 in combination with interferon gamma-1b. Expression levels are enhanced following exposure to interferon alphacon 1 in combination with interferon gamma-1b as compared to interferon alphacon 1 alone.

Table 3 shows genes correlated with direct antiviral effects in A549 cells.

TABLE 3
Genes correlated with direct antiviral effects in A549 cells

| | | | EC₅₀ cor- | |
|---|---|---|---|---|
| Description | Process | Gene | relation¹ | Probe³ |
| Antiviral/ | Undefined | G1P2/ | A, B | 205483_s_at |
| Immune | | ISG15 | | |
| | | G1P3 | A | 204415_at |
| | | IFI16 | A | 208966_x_at |
| | | IFI27 | A, B | 202411_at |
| | | IFI35 | B | 209417_s_at |
| | | IFI44 | A, B | 214453_s_at |
| | | IFIT1 | A | 203153_at |
| | | IFIT4 | B | 204747_at |
| | | IFIT4 | B | 229450_at |
| | | IFIT5 | A | 203595_s_at |
| | | IFITM1 | A, B | 201601_x_at |
| | | IFITM1 | A, B | 214022_s_at |
| | | IFITM2 | A, B | 201315_x_at |
| | | IFITM3 | B | 212203_x_at |
| | | SAMHD1 | B | 204502_at |
| | Classic | MX1 | A, B | 202086_at |
| | antiviral | | | |
| | | OAS1 | A | 202869_at |
| | | OAS1 | A | 205552_s_at |
| | | OAS2 | B | 204972_at |
| | | OAS3 | A, B | 218400_at |
| | | OASL | B | 210797_s_at |
| | | PRKR | A | 204211_x_at |
| | Antigen | HLA-C | B | 214459_x_at |
| | presentation | | | |
| | | HLA-F | B | 221875_x_at |
| | | PSME1 | B | 200814_at |
| | | PSME2 | B | 201762_s_at |
| | | UBL3 | B | 201535_at |
| | | USP18 | A, B | 219211_at |
| | Recruitment/ | BST2 | B | 201641_at |
| | Activation | | | |
| RNA-related | RNA | FLJ20035 | A, B | 218986_s_at |
| | helicase | | | |
| | | MDA5 | A, B | 219209_at |
| | | RIG-I | A | 222793_at |
| | | RIG-I | A | 242961_x_at |
| | Ribonuclease | ISG20 | B | 204698_at |
| | | ISG20 | B | 33304_at |
| | | PNPT1 | A | 225291_at |
| | Deaminase | ADAR | A | 201786_s_at |
| | RNA | ADPRHL1 | A, B | 1553163_at |
| | hydrolyase | | | |
| Signaling | Signal | ASB3 | A | 224524_s_at |
| | transduction | | | |
| | Transcription | HIC2 | B | 212966_at |
| | | IRF7 | A | 208436_s_at |
| | | ISGF3-γ | A, B | 203882_at |
| | | SP100 | A, B | 202864_s_at |
| | | SP110 | A | 208012_x_at |
| | | SP110 | A | 209761_s_at |
| | | SP110 | A, B | 209762_x_at |
| | | SP110 | A, B | 223980_s_at |
| | | STAT2 | B | 225636_at |
| | | ZNF347 | A | 238819_at |
| Cell Growth/ | Apoptosis | HSXIAPAF1 | B | 228617_at |
| Viability | | | | |
| | | PLSCR1 | A | 202430_s_at |
| | | PLSCR1 | A | 202446_s_at |
| | | PLSCR1 | A | 241916_at |
| | | SCOTIN | A, B | 222986_s_at |
| | Cell | FXYD5 | B | 218084_x_at |
| | adhesion | | | |
| Protein | Protein | FKBP11 | B | 228308_at |
| metabolism | folding | GRPEL1 | B | 212434_at |
| | Protein | FLJ20637 | A | 219352_at |
| | degradation | | | |
| Other | Unkown | — | A | 1565635_at |
| | | — | A, B | 230314_at |
| | | — | B | 235964_x_at |
| | | — | A | 244628_at |
| | | — | B | 228567_at |
| | | — | B | 238327_at |
| | | — | B | 239784_at |
| | | BAL | B | 223220_s_at |
| | | BAL | B | 227807_at |
| | | C1orf29 | A, B | 204439_at |
| | | C20orf18 | B | 221827_at |
| | | DKFZp434D1 | | |
| | | 77 | B | 236273_at |
| | | FLJ20073 | A | 219691_at |
| | | FLJ20073 | A | 228531_at |
| | | FLJ38348 | A | 213294_at |
| | | FLJ38348 | A | 238743_at |
| | | HOXB7 | A | 216973_s_at |
| | | KIAA1268 | B | 224701_at |
| | | LOC129607 | B | 226702_at |
| | | LOC253827 | B | 225782_at |
| | | MGC4054 | A | 220444_at |
| | | NA | B | 234987_at |
| | | NA | B | 235529_x_at |
| | | NPC2 | B | 200701_at |
| | | PCTAIRE2B | | |
| | | P | A | 213361_at |
| | | TRIM14 | A | 203148_s_at |
| | | UNC93B1 | A | 220998_s_at |
| | | WHSC1 | B | 222777_s_at |
| | Hepatic | MGST1 | B | 239001_at |
| | metabolism | | | |
| | Methyl | METTL2 | A | 221079_s_at |
| | transferase | | | |
| ¹A = equal expression following treatment of A549 cells with EC₅₀ IFN alfacon-1 (5.3 pg/mL), EC₅₀ IFN γ-1b (291.5 pg/mL), or IFN alfacon-1 and IFN γ-1b 1:60 combination required for EC₅₀ (1.1 pg/mL and 65.1 pg/mL, respectively); B = distinct expression level enhanced in A549 cells treated with IFN alfacon-1 and IFN γ-1b 1:60 combination required for EC₅₀ (1.1 pg/mL and 65.1 pg/mL, respectively) | | | | |
| # relative to A549 cells treated with IFN alfacon-1 or IFN γ-1b individually at the same dose. | | | | |

²Statistically different expression level in Huh7 liver-derived cells treated with a Cₘₐₓ dose of IFN alfacon-1 plus IFN γ-1b relative to IFN alfacon-1 alone (Welsh T-test, Benjamini and Hochberg adjusted p <0.05)., See text.
³Affymetfix U133 plus 2.0 microarray probe set ID

Table 4 shows quantitative enhancement of expression levels in select events in both A549 cells and in Huh7 cells after treatment with a combination of interferon alphacon 1 and interferon gamma-1b as compared to interferon alphacon 1 alone.

Table 5 shows the effect of interferon alphacon or the combination of interferon alphacon and interferon-gamma 1b on the expression ratios of several antiviral genes in hepatocyte-derived cells. Gene expression of the antiviral gene set is increased upon exposure to the interferon alphacon 1 and interferon-gamma 1b combination as compared to exposure to interferon alphacon 1 alone.

TABLE 5
Genes showing statistically significant expression differences in Huh7 cells with Cₘₐₓ IFN alfacon-1 plus Cₘₐₓ IFN γ-1b
relative to Cₘₐₓ IFN alfacon-1 alone¹

| | | | | | | Corrected | COMBO = IFN | |
|---|---|---|---|---|---|---|---|---|
| Description | Process | Gene | COMBO^{2,3} | IFN alfacon-1² | Change⁴ | p value⁵ | γ-1b⁶ | Probe⁷ |
| Immune system | Antigen presentation | HLA-E | 4.7 +/− 0.3 | 1.0 +/− 0.2 | 4.81 | 8.7E−03 | NO | 200905_x_at |
| | | HLA-B | 5.4 +/− 0.2 | 1.5 +/− 0.2 | 3.58 | 6.6E−03 | NO | 208729_x_at |
| | | HLA-B | 7.0 +/− 0.3 | 2.0 +/− 0.2 | 3.49 | 2.3E−03 | NO | 209140_x_at |
| | | HLA-B | 4.9 +/− 0.4 | 1.8 +/− 0.1 | 2.73 | 3.1E−04 | NO | 211911_x_at |
| | | HLA-E | 2.5 +/− 0.1 | 1.0 +/− 0.2 | 2.58 | 1.9E−02 | | 217456_x_at |
| | | HLA-C | 4.7 +/− 0.3 | 1.8 +/− 0.2 | 2.55 | 5.1E−03 | NO | 214459_x_at |
| | | HLA-A | 4.4 +/− 0.1 | 1.7 +/− 0.1 | 2.55 | 2.3E−03 | NO | 215313_x_at |
| | | HLA-G | 3.6 +/− 0.4 | 1.5 +/− 0.1 | 2.48 | 1.1E−03 | NO | 211529_x_at |
| | | HLA-C | 4.2 +/− 0.2 | 1.9 +/− 0.3 | 2.21 | 1.8E−02 | NO | 216526_x_at |
| | | HLA-C | 4.9 +/− 0.3 | 2.2 +/− 0.4 | 2.18 | 2.8E−02 | NO | 208812_x_at |
| | | HLA-A | 4.8 +/− 0.5 | 2.3 +/− 0.3 | 2.08 | 7.4E−03 | NO | 213932_x_at |
| | | HLA-G | 3.3 +/− 0.3 | 1.6 +/− 0.2 | 2.08 | 2.1E−03 | NO | 211528_x_at |
| | | HLA-G | 3.0 +/− 0.3 | 1.5 +/− 0.3 | 2.01 | 3.6E−02 | NO | 211530_x_at |
| | | HLA-F | 2.2 +/− 0.1 | 1.1 +/− 0.1 | 1.96 | 7.6E−03 | NO | 204806_x_at |
| | | HLA-F | 2.3 +/− 0.1 | 1.3 +/− 0.1 | 1.81 | 2.3E−03 | NO | 221875_x_at |
| | | B2M | 1.8 +/− 0.1 | 1.3 +/− 0.0 | 1.34 | 5.3E−04 | | 216231_s_at |
| | Antigen processing | PSMB8 | 101.8 +/− 9.9 | 1.5 +/− 1.0 | 65.91 | 1.2E−02 | NO | 209040_s_at |
| | | PSMB9 | 20.3 +/− 2.2 | 1.4 +/− 0.2 | 14.05 | 6.5E−04 | | 204279_at |
| | | TAP1 | 23.4 +/− 1.3 | 2.2 +/− 0.6 | 10.87 | 7.4E−03 | NO | 202307_s_at |
| | | UBD | 13.2 +/− 0.8 | 2.0 +/− 0.1 | 6.52 | 1.1E−05 | NO | 205890_s_at |
| | | UBE1L | 9.2 +/− 0.7 | 1.6 +/− 0.1 | 5.57 | 5.7E−03 | | 203281_s_at |
| | | PSMB10 | 3.8 +/− 0.3 | 1.0 +/− 0.2 | 3.80 | 8.1E−03 | | 202659_at |
| | | TAPBP-R | 6.0 +/− 0.6 | 1.7 +/− 0.4 | 3.54 | 1.2E−02 | | 218746_at |
| | | UBE2L6 | 6.7 +/− 0.2 | 2.1 +/− 0.1 | 3.20 | 1.4E−03 | NO | 201649_at |
| | | TAPBP-R | 4.3 +/− 0.6 | 1.5 +/− 0.2 | 2.83 | 9.5E−04 | | 218747_s_at |
| | | TAPBP | 5.8 +/− 0.3 | 2.3 +/− 0.1 | 2.50 | 6.3E−05 | NO | 208829_at |
| | | PSME2 | 3.1 +/− 0.3 | 1.7 +/− 0.3 | 1.84 | 1.4E−02 | | 201762_s_at |
| | | PSMA3 | 1.2 +/− 0.1 | 1.0 +/− 0.0 | 1.19 | 4.7E−03 | | 201532_at |
| | Undefined | GBP1 | 24.5 +/− 1.4 | 2.4 +/− 0.7 | 10.19 | 1.2E−02 | NO | 202269_x_at |
| | | GBP1 | 13.1 +/− 0.5 | 1.4 +/− 1.2 | 9.43 | 4.7E−02 | NO | 202270_at |
| | | IFI27 | 15.2 +/− 1.6 | 2.9 +/− 0.3 | 5.31 | 6.5E−04 | NO | 202411_at |
| | | IFITM1 | 13.1 +/− 0.5 | 2.8 +/− 0.7 | 4.68 | 1.5E−02 | NO | 214022_s_at |
| | | IFITM1 | 67.9 +/− 1.9 | 20.4 +/− 1.8 | 3.33 | 4.0E−03 | NO | 201601_x_at |
| | | 1FI30 | 3.1 +/− 0.3 | 1.5 +/− 0.3 | 2.14 | 2.2E−02 | | 201422_at |
| | | 1FI35 | 3.7 +/− 0.2 | 1.9 +/− 0.3 | 1.94 | 1.9E−02 | NO | 209417_s_at |
| | | IFITM3 | 110.8 +/− 2.9 | 62.3 +/− 1.0 | 1.78 | 2.3E−05 | NO | 212203_x_at |
| | | IFITM2 | 11.3 +/− 0.3 | 6.5 +/− 1.1 | 1.74 | 4.7E−02 | NO | 201315_x_at |
| | | IFIT4 | 3.7 +/− 0.3 | 2.3 +/− 0.1 | 1.65 | 1.0E−03 | NO | 204747_at |
| | Complement activation | C4A | 10.7 +/− 0.9 | 1.1 +/− 0.5 | 9.58 | 2.2E−02 | | 208451_s_at |
| | | C1S | 10.7 +/− 0.7 | 1.7 +/− 0.2 | 6.34 | 1.5E−03 | NO | 208747_s_at |
| | | — | 8.4 +/− 0.2 | 1.5 +/− 0.3 | 5.81 | 1.1E−02 | | 212067_s_at |
| | | IF | 3.0 +/− 0.5 | 1.2 +/− 0.1 | 2.61 | 1.4E−03 | | 203854_at |
| | | HF1 | 2.6 +/− 0.2 | 1.1 +/− 0.2 | 2.42 | 8.1E−03 | NO | 213800_at |
| | | SERPING1 | 2.2 +/− 0.0 | 1.1 +/− 0.1 | 2.04 | 1.2E−02 | NO | 200986_at |
| | | BF | 3.7 +/− 0.4 | 2.0 +/− 0.1 | 1.87 | 2.1E−03 | NO | 202357_s_at |
| | | C3 | 2.8 +/− 0.2 | 1.7 +/− 0.1 | 1.60 | 2.9E−03 | NO | 217767_at |
| | Recruitment/activation | BST2 | 17.0 +/− 1.4 | 1.2 +/− 0.1 | 14.05 | 1.1E−05 | NO | 201641_at |
| | | LGALS3BP | 9.8 +/− 1.0 | 2.8 +/− 0.3 | 3.51 | 5.3E−04 | NO | 200923_at |
| | | IL15 | 4.4 +/− 1.2 | 1.3 +/− 0.0 | 3.26 | 8.7E−03 | | 205992_s_at |
| | | CSF1 | 2.9 +/− 0.1 | 1.4 +/− 0.3 | 2.10 | 4.5E−02 | NO | 209716_at |
| | | WARS | 2.2 +/− 0.1 | 1.1 +/− 0.2 | 2.01 | 3.0E−02 | | 200628_s_at |
| | Tentative immune | SLC15A3 | 17.7 +/− 3.4 | 1.0 +/− 0.4 | 17.92 | 7.4E−03 | NO | 219593_at |
| | | — | 9.0 +/− 0.9 | 4.0 +/− 0.4 | 2.26 | 2.3E−03 | NO | 216565_x_at |
| | | BTN3A3 | 3.7 +/− 0.2 | 1.6 +/− 0.4 | 2.24 | 3.8E−02 | | 38241_at |
| | | — | 3.3 +/− 0.4 | 1.8 +/− 0.3 | 1.85 | 1.0E−02 | NO | 217436_x_at |
| | | TCIRG1 | 1.6 +/− 0.1 | 1.2 +/− 0.1 | 1.33 | 2.3E−03 | | 204158_s_at |
| | Direct antiviral | ISG20 | 16.1 +/− 1.2 | 4.5 +/− 0.4 | 3.56 | 5.3E−04 | NO | 204698_at |
| | | OAS2 | 5.1 +/− 0.5 | 2.5 +/− 0.6 | 2.03 | 4.2E−02 | NO | 204972_at |
| | | ISG20 | 2.5 +/− 0.2 | 1.3 +/− 0.1 | 1.92 | 6.2E−03 | NO | 33304_at |
| | | OAS3 | 4.9 +/− 0.2 | 3.4 +/− 0.3 | 1.44 | 1.9E−02 | NO | 218400_at |
| Growth/ | Apoptosis | HSXIAPAF1 | 12.4 +/− 0.9 | 2.6 +/− 1.2 | 4.73 | 3.4E−02 | NO | 206133_at |
| viability | | CASP1 | 2.5 +/− 0.3 | 0.8 +/− 0.2 | 3.22 | 2.3E−02 | | 209970_x_at |
| | | CASP1 | 2.2 +/− 0.3 | 1.2 +/− 0.0 | 1.78 | 6.4E−03 | | 211366_x_at |
| | | TNFSF10 | 1.8 +/− 0.1 | 1.2 +/− 0.1 | 1.50 | 8.9E−03 | | 202688_at |
| | | TNFSF10 | 1.9 +/− 0.2 | 1.2 +/− 0.1 | 1.49 | 1.2E−02 | | 202687_s_at |
| | | NFKBIA | 1.2 +/− 0.0 | 1.5 +/− 0.1 | 0.80 | 3.6E−03 | | 201502_s_at |
| | | TNFSF9 | 1.1 +/− 0.1 | 1.4 +/− 0.0 | 0.80 | 2.3E−03 | | 206907_at |
| | Cell adhesion | NK4 | 20.5 +/− 1.2 | 5.1 +/− 0.6 | 4.05 | 2.5E−03 | NO | 203828_s_at |
| | | ICAM1 | 2.4 +/− 0.1 | 1.2 +/− 0.2 | 1.98 | 2.1E−02 | | 202638_s_at |
| | | ICAM1 | 2.3 +/− 0.3 | 1.2 +/− 0.1 | 1.86 | 4.9E−03 | | 215485_s_at |
| | | HABP2 | 2.3 +/− 0.1 | 1.3 +/− 0.2 | 1.86 | 2.1E−02 | NO | 206010_at |
| | | CSPG2 | 0.9 +/− 0.0 | 1.1 +/− 0.0 | 0.88 | 2.2E−02 | NO | 221731_x_at |
| | Cell cycle/proliferation | RARRES3 | 52.0 +/− 3.5 | 2.3 +/− 0.3 | 22.48 | 7.2E−04 | | 204070_at |
| | | LAMP3 | 14.1 +/− 1.2 | 7.0 +/− 0.9 | 2.02 | 6.8E−03 | NO | 205569_at |
| | | CDC25B | 1.3 +/− 0.0 | 1.0 +/− 0.1 | 1.28 | 3.2E−02 | | 201853_s_at |
| | | NBS1 | 1.2 +/− 0.0 | 1.0 +/− 0.0 | 1.23 | 8.8E−04 | | 202907_s_at |
| | Cytoskeleton | MARCKS | 1.0 +/− 0.0 | 1.1 +/− 0.0 | 0.921 | 2.3E−03 | NO | 201670_s_at |
| Signaling | Transcription | NMI | 3.7 +/− 0.1 | 1.4 +/− 0.2 | 2.58 | 1.1E−02 | NO | 203964_at |
| | | IRF1 | 2.8 +/− 0.3 | 1.2 +/− 0.1 | 2.36 | 1.6E−03 | | 202531_at |
| | | STAT2 | 2.8 +/− 0.2 | 1.2 +/− 0.3 | 2.24 | 3.5E−02 | NO | 205170_at |
| | | | | | | | | AFFX- |
| | | | | | | | | HUMISGF3A/ |
| | | ISGF3-γ | 8.2 +/− 0.6 | 4.2 +/− 0.4 | 1.95 | 3.8E−03 | NO | M97935_3_at |
| | | STAT1 | 12.3 +/− 0.9 | 6.8 +/− 1.0 | 1.83 | 1.9E−02 | NO | 209969_s_at |
| | | | | | | | | AFFX- |
| | | | | | | | | HUMISGF3A/ |
| | | ISGF3-γ | 6.6 +/− 0.3 | 3.7 +/− 0.2 | 1.77 | 1.4E−03 | NO | M97935_5_at |
| | | | | | | | | AFFX- |
| | | | | | | | | HUMISGF3A/ |
| | | ISGF3-γ | 7.2 +/− 0.2 | 4.3 +/− 0.2 | 1.67 | 6.0E−03 | NO | M97935_5_at |
| | | | | | | | | AFFX- |
| | | | | | | | | HUMISGF3A/ |
| | | ISGF3-γ | 4.4 +/− 0.1 | 2.8 +/− 0.2 | 1.57 | 7.2E−03 | NO | M97935_3_at |
| | | TEAD4 | 1.1 +/− 0.0 | 0.8 +/− 0.0 | 1.36 | 5.3E−03 | | 204281_at |
| | | ZNF337 | 0.9 +/− 0.1 | 0.7 +/− 0.0 | 1.28 | 4.5E−02 | | 37860_at |
| | | RCOR | 1.0 +/− 0.0 | 0.8 +/− 0.0 | 1.27 | 1.2E−03 | | 212612_at |
| | | ISGF3-γ | 14.1 +/− 1.2 | 11.4 +/− 0.6 | 1.24 | 1.9E−02 | NO | 203882_at |
| Other | Unknown | — | 3.2 +/− 2.0 | 0.3 +/− 0.0 | 10.27 | 6.0E−03 | | 213317_at |
| | | TRIM22 | 8.3 +/− 0.6 | 1.4 +/− 0.4 | 5.88 | 1.9E−02 | NO | 213293_s_at |
| | | — | 2.6 +/− 0.3 | 0.9 +/− 0.1 | 2.94 | 7.2E−04 | NO | 211456_x_at |
| | | — | 2.8 +/− 0.2 | 1.3 +/− 0.1 | 2.13 | 5.3E−04 | | 202637_s_at |
| | | — | 2.8 +/− 0.4 | 1.6 +/− 0.3 | 1.74 | 4.2E−02 | NO | 216336_x_at |
| | | FLJ11000 | 1.9 +/− 0.2 | 1.1 +/− 0.1 | 1.71 | 2.3E−03 | NO | 218999_at |
| | | NUCB1 | 1.9 +/− 0.1 | 1.2 +/− 0.1 | 1.57 | 1.4E−02 | | 200649_at |
| | | C11orf25 | 1.5 +/− 0.1 | 0.9 +/− 0.0 | 1.56 | 5.3E−04 | | 215241_at |
| | | TRIM31 | 1.5 +/− 0.1 | 1.1 +/− 0.1 | 1.42 | 1.0E−02 | | 215444_s_at |
| | | HNOEL-iso | 1.5 +/− 0.0 | 1.2 +/− 0.1 | 1.27 | 3.9E−02 | | 218162_at |
| | | TRIM31 | 1.5 +/− 0.1 | 1.2 +/− 0.0 | 1.25 | 7.6E−03 | | 208170_s_at |
| | | LEPROTL1 | 1.2 +/− 0.0 | 1.0 +/− 0.0 | 1.25 | 7.2E−04 | | 202594_at |
| | | HRASLS3 | 1.3 +/− 0.0 | 1.1 +/− 0.0 | 1.20 | 1.3E−03 | | 209531_at |
| | | CGI-109 | 1.0 +/− 0.0 | 0.9 +/− 0.0 | 1.16 | 9.5E−04 | NO | 209404_s_at |
| | | BEX1 | 0.8 +/− 0.0 | 1.1 +/− 0.0 | 0.76 | 2.0E−03 | | 218332_at |
| | Carb. & lipid related | APOL6 | 5.9 +/− 0.7 | 1.5 +/− 0.2 | 3.83 | 1.0E−03 | NO | 219716_at |
| | | ANXA1 | 1.7 +/− 0.1 | 1.3 +/− 0.1 | 1.31 | 2.2E−02 | NO | 201012_at |
| | | HMGCR | 1.1 +/− 0.1 | 0.9 +/− 0.0 | 1.20 | 1.0E−02 | | 202540_s_at |
| | | SORD | 0.9 +/− 0.0 | 1.0 +/− 0.0 | 0.90 | 1.1E−02 | NO | 201563_at |
| | | GYG2 | 0.7 +/− 0.0 | 0.8 +/− 0.0 | 0.84 | 1.6E−02 | NO | 215695_s_at |
| | | GBA3 | 0.8 +/− 0.0 | 1.0 +/− 0.0 | 0.82 | 1.8E−02 | NO | 219954_s_at |
| | | SOAT2 | 1.1 +/− 0.1 | 1.3 +/− 0.0 | 0.81 | 1.8E−02 | | 210677_at |
| | Protein degradation | CTSS | 3.5 +/− 0.2 | 1.2 +/− 0.2 | 2.94 | 7.4E−03 | NO | 202902_s_at |
| | | ARTS-1 | 3.1 +/− 0.2 | 1.7 +/− 0.2 | 1.85 | 5.8E−03 | | 209788_s_at |
| | | ARTS-1 | 2.6 +/− 0.2 | 1.5 +/− 0.0 | 1.76 | 7.2E−04 | | 210385_s_at |
| | | DNPEP | 1.8 +/− 0.1 | 1.1 +/− 0.2 | 1.75 | 3.3E−02 | | 201937_s_at |
| | | DNPEP | 1.6 +/− 0.1 | 1.0 +/− 0.1 | 1.52 | 8.9E−03 | | 38703_at |
| | | LAP3 | 2.1 +/− 0.0 | 1.7 +/− 0.1 | 1.29 | 8.7E−03 | NO | 217933_s_at |
| | Metal ion homeostasis | MT1X | 2.9 +/− 0.3 | 1.3 +/− 0.2 | 2.15 | 4.4E−03 | NO | 208581_x_at |
| | | MT2A | 2.3 +/− 0.1 | 1.4 +/− 0.1 | 1.69 | 7.5E−03 | NO | 212185_x_at |
| | | MT2A | 1.8 +/− 0.0 | 1.2 +/− 0.1 | 1.47 | 1.9E−02 | NO | 212859_x_at |
| | | MT1X | 1.7 +/− 0.1 | 1.2 +/− 0.0 | 1.44 | 9.5E−04 | NO | 204326_x_at |
| | Response to stress | SOD2 | 1.5 +/− 0.2 | 1.0 +/− 0.1 | 1.55 | 1.3E−02 | | 216841_s_at |
| | | SOD2 | 2.1 +/− 0.1 | 1.4 +/− 0.1 | 1.50 | 7.5E−03 | | 215223_s_at |
| | | NDRG4 | 0.8 +/− 0.0 | 1.2 +/− 0.1 | 0.62 | 4.4E−03 | | 209159_s_at |
| | Mitochondrial | UCP2 | 1.4 +/− 0.1 | 1.2 +/− 0.0 | 1.14 | 8.7E−03 | | 208998_at |
| | | ABLIM1 | 0.8 +/− 0.0 | 0.9 +/− 0.0 | 0.91 | 1.4E−02 | NO | 200965_s_at |
| | Cell activation | LGALS8 | 1.2 +/− 0.0 | 1.1 +/− 0.0 | 1.09 | 3.0E−02 | | 208934_s_at |
| | Creatine kinase | CKB | 1.2 +/− 0.0 | 1.1 +/− 0.0 | 1.09 | 3.0E−03 | NO | 200884_at |
| | Lysine hydroxylase | PLOD2 | 1.3 +/− 0.0 | 1.1 +/− 0.1 | 1.23 | 3.0E−02 | | 202620_s_at |
| | Neurotensin | NTS | 1.0 +/− 0.1 | 1.2 +/− 0.0 | 0.86 | 2.3E−02 | | 206291_at |
| | Nucleic acid binding | FLJ22693 | 2.3 +/− 0.1 | 1.8 +/− 0.1 | 1.29 | 1.9E−02 | NO | 218543_s_at |
| | Nucleolar protein | ANKT | 0.9 +/− 0.0 | 1.0 +/− 0.0 | 0.95 | 1.7E−03 | NO | 218039_at |
| | Prostaglandin | HPGD | 1.5 +/− 0.1 | 1.2 +/− 0.1 | 1.21 | 4.2E−02 | NO | 203914_x_at |
| | metabolism | | | | | | | |
| | Translation | EIF4A2 | 1.1 +/− 0.0 | 1.0 +/− 0.0 | 1.11 | 1.7E−03 | NO | 200912_s_at |
| | tRNA processing | FLJ10140 | 0.7 +/− 0.0 | 1.2 +/− 0.1 | 0.60 | 1.8E−03 | NO | 213634_s_at |
| | Wound healing | SERPINE1 | 1.8 +/− 0.0 | 1.4 +/− 0.1 | 1.30 | 2.3E−02 | NO | 202627_s_at |
| | Zinc finger | ZDHHC4 | 0.9 +/− 0.0 | 1.0 +/− 0.0 | 0.89 | 6.9E−04 | | 220261_s_at |

¹Cₘₐₓ concentrations are IFN alfacon-1 = 80 pg/mL, IFN γ-1b = 300 pg/mL
²Expressed as signal in treated cells/signal in untreated cells
³IFN alfacon-1 plus IFN γ-1b
⁴Change in expression level following combined IFN alfacon-1 plus IFN γ-1b treatment relative to IFN alfacon-1 treatment expressed as ratio
⁵Benjamini and Hochberg multiple test corrected p value
⁶NO indicates statistically significant expression differences between cells treated with IFN alfacon-1 plus IFN γ-1b and cells treated with IFN γ-1b alone (Welsh T-test, Benjamini and Hochberg adjusted p < 0.05)
⁷Affymetrix U133A microarray probe set ID

Table 6 further shows the effect of exposure of maximal human plasma concentrations of interferon alphacon 1 or interferon gamma-1b or the combination of interferon alphacon 1 and interferon-gamma 1b on the expression ratios of several antiviral genes in hepatocyte-derived cells. Gene expression of the antiviral gene set is increased upon exposure to the interferon alphacon 1 and interferon-gamma 1b combination as compared to exposure to interferon alphacon 1 or interferon gamma-1b alone.

TABLE 6
Genes showing statistically significant differences in expression in Huh7 cells following exposure to maximum human plasma concentrations
of IFN alfacon-1, IFN γ-1b, or both

| | | | | | | Expression change ratio in | | |
|---|---|---|---|---|---|---|---|---|
| | | | | | | different treatment regimes² | | |
| | | | | | | | | IFN γ- |
| | | | Expression ratio¹ | | | COMBO⁵/IFN | COMBO⁵/ | 1b/IFN |
| Gene Symbol | Probe Set ID³ | OMIM⁴ | Combination⁵ | IFN γ-1b | IFN alfacon-1 | alfacon-1 | IFN γ-1b | alfacon-1 |
| PSMB8 | 209040_s_at | 177046 | 101.8 +/− 9.86 | 78.6 +/− 3.47 | 1.5 +/− 0.95 | 65.91 | 1.30 | 50.84 |
| RARRES3 | 204070_at | 605092 | 52.0 +/− 3.48 | 50.6 +/− 7.34 | 2.3 +/− 0.28 | 22.48 | 1.03 | 21.85 |
| SLC15A3 | 219593_at | — | 17.7 +/− 3.43 | 6.5 +/− 2.85 | 1.0 +/− 0.39 | 17.92 | 2.74 | 6.55 |
| PSMB9 | 204279_at | 177045 | 20.3 +/− 2.17 | 16.7 +/− 1.80 | 1.4 +/− 0.21 | 14.05 | 1.21 | 11.58 |
| BST2 | 201641_at | 600534 | 17.0 +/− 1.41 | 3.4 +/− 2.09 | 1.2 +/− 0.05 | 14.05 | 4.98 | 2.82 |
| TAP1 | 202307_s_at | 170260 | 23.4 +/− 1.30 | 17.6 +/− 0.98 | 2.2 +/− 0.55 | 10.87 | 1.33 | 8.18 |
| — | 213317_at | — | 3.2 +/− 1.96 | 2.4 +/− 0.93 | 0.3 +/− 0.05 | 10.27 | 1.31 | 7.83 |
| GBP1 | 202269_x_at | 600411 | 24.5 +/− 1.42 | 19.4 +/− 1.55 | 2.4 +/− 0.68 | 10.19 | 1.27 | 8.05 |
| C4A | 208451_s_at | 120810 | 10.7 +/− 0.89 | 9.7 +/− 0.26 | 1.1 +/− 0.46 | 9.58 | 1.10 | 8.74 |
| GBP1 | 202270_at | 600411 | 13.1 +/− 0.49 | 9.4 +/− 1.56 | 1.4 +/− 1.16 | 9.43 | 1.39 | 6.77 |
| CASP1 | 211368_s_at | 147678 | 25.9 +/− 3.30 | 28.3 +/− 2.64 | 3.5 +/− 3.40 | 7.36 | 0.92 | 8.02 |
| UBD | 205890_s_at | 606050 | 13.2 +/− 0.80 | 11.1 +/− 0.80 | 2.0 +/− 0.08 | 6.52 | 1.19 | 5.47 |
| C1S | 208747_s_at | 120580 | 10.7 +/− 0.71 | 9.2 +/− 0.32 | 1.7 +/− 0.21 | 6.34 | 1.16 | 5.48 |
| TRIM22 | 213293_s_at | 606559 | 8.3 +/− 0.58 | 2.2 +/− 0.76 | 1.4 +/− 0.43 | 5.88 | 3.72 | 1.58 |
| — | 212067_s_at | — | 8.4 +/− 0.20 | 7.6 +/− 0.50 | 1.5 +/− 0.30 | 5.81 | 1.11 | 5.22 |
| IFI27 | 202411_at | 600009 | 15.2 +/− 1.63 | 1.3 +/− 0.58 | 2.9 +/− 0.34 | 5.31 | 11.29 | 0.47 |
| HLA-E | 200905_x_at | 143010 | 4.7 +/− 0.34 | 3.8 +/− 0.21 | 1.0 +/− 0.19 | 4.81 | 1.24 | 3.87 |
| HSXIAPAF1 | 206133_at | 606717 | 12.4 +/− 0.86 | 2.2 +/− 0.53 | 2.6 +/− 1.17 | 4.73 | 5.60 | 0.85 |
| IFITM1 | 214022_s_at | 604456 | 13.1 +/− 0.45 | 3.9 +/− 0.22 | 2.8 +/− 0.69 | 4.68 | 3.37 | 1.39 |
| BTN3A2 | 212613_at | — | 12.3 +/− 4.64 | 10.8 +/− 3.14 | 2.6 +/− 2.18 | 4.65 | 1.14 | 4.08 |
| HLA-C | 211799_x_at | 142840 | 13.4 +/− 1.45 | 5.6 +/− 1.40 | 3.1 +/− 2.87 | 4.33 | 2.42 | 1.79 |
| NNMT | 202237_at | 600008 | 3.5 +/− 0.19 | 3.5 +/− 0.27 | 0.8 +/− 0.46 | 4.25 | 1.00 | 4.24 |
| NK4 | 203828_s_at | 606001 | 20.5 +/− 1.23 | 17.3 +/− 1.62 | 5.1 +/− 0.60 | 4.05 | 1.18 | 3.42 |
| APOL6 | 219716_at | 607256 | 5.9 +/− 0.69 | 4.0 +/− 0.53 | 1.5 +/− 0.21 | 3.82 | 1.48 | 2.59 |
| PSMB10 | 202659_at | 176847 | 3.8 +/− 0.25 | 4.1 +/− 0.30 | 1.0 +/− 0.17 | 3.80 | 0.93 | 4.07 |
| HLA-B | 208729_x_at | 142830 | 5.4 +/− 0.24 | 3.6 +/− 0.44 | 1.5 +/− 0.21 | 3.58 | 1.51 | 2.37 |
| ISG20 | 204698_at | 604533 | 16.1 +/− 1.22 | 8.8 +/− 0.87 | 4.5 +/− 0.35 | 3.56 | 1.84 | 1.94 |
| TAPBP-R | 218746_at | 607081 | 6.0 +/− 0.63 | 4.7 +/− 0.89 | 1.7 +/− 0.42 | 3.54 | 1.28 | 2.77 |
| TEAD | 204281_at | | 0.8 +/− 0.11 | 0.3 +/− 0.45 | 0.2 +/− 0.17 | 3.53 | 2.62 | 1.35 |
| LGALS3BP | 200923_at | 600626 | 9.8 +/− 0.98 | 4.5 +/− 0.45 | 2.8 +/− 0.26 | 3.51 | 2.21 | 1.59 |
| HLA-B | 209140_x_at | 142830 | 7.0 +/− 0.32 | 3.5 +/− 0.25 | 2.0 +/− 0.19 | 3.49 | 1.97 | 1.77 |
| UBE1L | 203281_s_at | 191325 | 4.9 +/− 0.98 | 2.0 +/− 2.78 | 1.4 +/− 0.90 | 3.43 | 2.50 | 2.59 |
| IFITM1 | 201601_x_at | 604456 | 67.9 +/− 1.91 | 21.5 +/− 1.29 | 20.4 +/− 1.82 | 3.33 | 3.15 | 1.06 |
| IL15 | 205992_s_at | 600554 | 4.4 +/− 1.15 | 4.1 +/− 0.14 | 1.3 +/− 0.03 | 3.26 | 1.06 | 3.06 |
| CASP1 | 209970_x_at | 147678 | 2.5 +/− 0.26 | 2.5 +/− 0.04 | 0.8 +/− 0.23 | 3.22 | 1.02 | 3.16 |
| UBE2L6 | 201649_at | 603890 | 6.7 +/− 0.15 | 5.6 +/− 0.29 | 2.1 +/− 0.11 | 3.20 | 1.18 | 2.71 |
| — | 211456_x_at | — | 2.6 +/− 0.29 | 1.4 +/− 0.45 | 0.9 +/− 0.09 | 2.94 | 1.92 | 1.53 |
| CTSS | 202902_s_at | 116845 | 3.5 +/− 0.22 | 2.6 +/− 0.17 | 1.2 +/− 0.18 | 2.93 | 1.32 | 2.22 |
| TAPBP-R | 218747_s_at | 607081 | 4.3 +/− 0.56 | 4.8 +/− 0.45 | 1.5 +/− 0.15 | 2.83 | 0.90 | 3.15 |
| HLA-B | 211911_x_at | 142830 | 4.9 +/− 0.39 | 2.9 +/− 0.11 | 1.8 +/− 0.07 | 2.73 | 1.68 | 1.63 |
| IF | 203854_at | 217030 | 3.0 +/− 0.45 | 2.7 +/− 0.34 | 1.2 +/− 0.08 | 2.61 | 1.12 | 2.34 |
| NMI | 203964_at | 603525 | 3.7 +/− 0.13 | 2.9 +/− 0.12 | 1.4 +/− 0.19 | 2.58 | 1.28 | 2.02 |
| HLA-E | 217456_x_at | 143010 | 2.5 +/− 0.14 | 2.2 +/− 0.31 | 1.0 +/− 0.19 | 2.57 | 1.12 | 2.31 |
| HLA-C | 214459_x_at | 142840 | 4.7 +/− 0.31 | 2.8 +/− 0.09 | 1.8 +/− 0.22 | 2.55 | 1.68 | 1.52 |
| HLA-A | 215313_x_at | 142800 | 4.4 +/− 0.09 | 3.2 +/− 0.19 | 1.7 +/− 0.10 | 2.55 | 1.35 | 1.89 |
| TAPBP | 208829_at | 601962 | 5.8 +/− 0.28 | 4.9 +/− 0.43 | 2.3 +/− 0.06 | 2.50 | 1.19 | 2.11 |
| HLA-G | 211529_x_at | 142871 | 3.6 +/− 0.38 | 2.6 +/− 0.33 | 1.5 +/− 0.14 | 2.48 | 1.40 | 1.78 |
| HF1 | 213800_at | 134370 | 2.6 +/− 0.21 | 2.1 +/− 0.16 | 1.1 +/− 0.16 | 2.41 | 1.27 | 1.90 |
| IRF1 | 202531_at | 147575 | 2.8 +/− 0.28 | 2.8 +/− 0.14 | 1.2 +/− 0.13 | 2.36 | 1.02 | 2.31 |
| — | 216565_x_at | — | 9.0 +/− 0.92 | 3.0 +/− 0.22 | 4.0 +/− 0.43 | 2.26 | 2.96 | 0.76 |
| STAT2 | 205170_at | 600556 | 2.8 +/− 0.15 | 1.4 +/− 0.17 | 1.2 +/− 0.28 | 2.24 | 2.00 | 1.12 |
| BTN3A3 | 38241_at | — | 3.7 +/− 0.15 | 3.5 +/− 0.62 | 1.6 +/− 0.36 | 2.24 | 1.06 | 2.11 |
| HLA-C | 216526_x_at | 142840 | 4.2 +/− 0.18 | 2.6 +/− 0.06 | 1.9 +/− 0.28 | 2.21 | 1.64 | 1.35 |
| HLA-C | 208812_x_at | 142840 | 4.9 +/− 0.32 | 3.1 +/− 0.27 | 2.2 +/− 0.44 | 2.18 | 1.58 | 1.38 |
| MT1X | 208581_x_at | 156359 | 2.9 +/− 0.31 | 1.9 +/− 0.15 | 1.3 +/− 0.17 | 2.15 | 1.51 | 1.42 |
| IFI30 | 201422_at | 604664 | 3.1 +/− 0.29 | 2.8 +/− 0.15 | 1.5 +/− 0.28 | 2.14 | 1.13 | 1.90 |
| — | 202637_s_at | — | 2.8 +/− 0.23 | 2.7 +/− 0.16 | 1.3 +/− 0.07 | 2.13 | 1.04 | 2.04 |
| CSF1 | 209716_at | 120420/// | 2.9 +/− 0.14 | 2.1 +/− 0.17 | 1.4 +/− 0.34 | 2.10 | 1.40 | 1.51 |
| | | 120420 | | | | | | |
| HLA-A | 213932_x_at | 142800 | 4.8 +/− 0.46 | 3.5 +/− 0.62 | 2.3 +/− 0.31 | 2.08 | 1.38 | 1.50 |
| HLA-G | 211528_x_at | 142871 | 3.3 +/− 0.31 | 2.3 +/− 0.27 | 1.6 +/− 0.15 | 2.08 | 1.42 | 1.46 |
| SERPING1 | 200986_at | 606860 | 2.2 +/− 0.01 | 2.0 +/− 0.08 | 1.1 +/− 0.10 | 2.04 | 1.13 | 1.82 |
| OAS2 | 204972_at | 603350 | 5.1 +/− 0.52 | 1.1 +/− 0.19 | 2.5 +/− 0.61 | 2.03 | 4.41 | 0.46 |
| LAMP3 | 205569_at | 605883 | 14.1 +/− 1.15 | 4.2 +/− 0.29 | 7.0 +/− 0.88 | 2.02 | 3.35 | 0.60 |
| HLA-G | 211530_x_at | 142871 | 3.0 +/− 0.27 | 2.4 +/− 0.27 | 1.5 +/− 0.33 | 2.01 | 1.24 | 1.62 |
| WARS | 200628_s_at | 191050 | 2.2 +/− 0.11 | 2.1 +/− 0.09 | 1.1 +/− 0.19 | 2.01 | 1.02 | 1.96 |
| ICAM1 | 202638_s_at | 147840 | 2.4 +/− 0.10 | 2.2 +/− 0.13 | 1.2 +/− 0.17 | 1.98 | 1.11 | 1.79 |
| HLA-F | 204806_x_at | 143110 | 2.2 +/− 0.10 | 1.6 +/− 0.14 | 1.1 +/− 0.11 | 1.95 | 1.38 | 1.41 |
| ISGF3-γ | AFFX- | — | 8.2 +/− 0.58 | 6.7 +/− 0.51 | 4.2 +/− 0.44 | 1.95 | 1.24 | 1.58 |
| | HUMISGF3A/M97935_{—} | | | | | | | |
| | 3_at | | | | | | | |
| IFI35 | 209417_s_at | 600735 | 3.7 +/− 0.16 | 2.0 +/− 0.16 | 1.9 +/− 0.26 | 1.94 | 1.85 | 1.05 |
| ISG20 | 33304_at | 604533 | 2.5 +/− 0.14 | 1.7 +/− 0.06 | 1.3 +/− 0.12 | 1.92 | 1.45 | 1.33 |
| BF | 202357_s_at | 138470 | 3.7 +/− 0.42 | 2.1 +/− 0.30 | 2.0 +/− 0.11 | 1.86 | 1.76 | 1.06 |
| ICAM1 | 215485_s_at | 147840 | 2.3 +/− 0.33 | 2.1 +/− 0.15 | 1.2 +/− 0.14 | 1.86 | 1.09 | 1.71 |
| HABP2 | 206010_at | 603924 | 2.3 +/− 0.05 | 1.5 +/− 0.25 | 1.3 +/− 0.16 | 1.86 | 1.53 | 1.22 |
| — | 217436_x_at | — | 3.3 +/− 0.43 | 2.1 +/− 0.32 | 1.8 +/− 0.27 | 1.85 | 1.54 | 1.21 |
| ARTS-1 | 209788_s_at | 606832 | 3.1 +/− 0.19 | 2.9 +/− 0.13 | 1.7 +/− 0.15 | 1.85 | 1.08 | 1.71 |
| PSME2 | 201762_s_at | 602161 | 3.1 +/− 0.33 | 2.9 +/− 0.13 | 1.7 +/− 0.25 | 1.84 | 1.06 | 1.74 |
| STAT1 | 209969_s_at | 600555 | 12.3 +/− 0.92 | 8.8 +/− 0.60 | 6.8 +/− 1.04 | 1.83 | 1.40 | 1.31 |
| HLA-F | 221875_x_at | 143110 | 2.3 +/− 0.09 | 1.6 +/− 0.11 | 1.3 +/− 0.08 | 1.81 | 1.45 | 1.25 |
| CASP1 | 211366_x_at | 147678 | 2.2 +/− 0.26 | 2.0 +/− 0.06 | 1.2 +/− 0.03 | 1.78 | 1.10 | 1.62 |
| IFITM3 | 212203_x_at | 605579 | 110.8 +/− 2.90 | 26.8 +/− 2.12 | 62.3 +/− 1.02 | 1.78 | 4.13 | 0.43 |
| ISGF3-γ | AFFX- | — | 6.6 +/− 0.25 | 5.4 +/− 0.24 | 3.7 +/− 0.19 | 1.77 | 1.21 | 1.46 |
| | HUMISGF3A/M97935_{—} | | | | | | | |
| | 5_at | | | | | | | |
| ARTS-1 | 210385_s_at | 606832 | 2.6 +/− 0.17 | 2.3 +/− 0.17 | 1.5 +/− 0.04 | 1.76 | 1.13 | 1.56 |
| DNPEP | 201937_s_at | — | 1.8 +/− 0.12 | 1.9 +/− 0.10 | 1.1 +/− 0.17 | 1.75 | 0.98 | 1.78 |
| — | 216336_x_at | — | 2.8 +/− 0.37 | 2.0 +/− 0.24 | 1.6 +/− 0.33 | 1.74 | 1.38 | 1.27 |
| IFITM2 | 201315_x_at | 605578 | 11.3 +/− 0.31 | 4.1 +/− 0.72 | 6.5 +/− 1.12 | 1.74 | 2.75 | 0.63 |
| FLJ11000 | 218999_at | — | 1.9 +/− 0.17 | 1.0 +/− 0.11 | 1.1 +/− 0.09 | 1.71 | 1.82 | 0.94 |
| MT2A | 212185_x_at | 156360 | 2.3 +/− 0.08 | 1.8 +/− 0.13 | 1.4 +/− 0.10 | 1.69 | 1.28 | 1.32 |
| ISGF3-γ | AFFX- | — | 7.2 +/− 0.21 | 5.6 +/− 0.49 | 4.3 +/− 0.23 | 1.67 | 1.28 | 1.30 |
| | HUMISGF3A/M97935_{—} | | | | | | | |
| | 5_at | | | | | | | |
| IFIT4 | 204747_at | 604650 | 3.7 +/− 0.26 | 2.2 +/− 0.16 | 2.3 +/− 0.12 | 1.65 | 1.68 | 0.98 |
| C3 | 217767_at | 120700 | 2.8 +/− 0.24 | 2.0 +/− 0.12 | 1.7 +/− 0.05 | 1.60 | 1.39 | 1.15 |
| NUCB1 | 200649_at | 601323 | 1.9 +/− 0.07 | 1.7 +/− 0.22 | 1.2 +/− 0.11 | 1.57 | 1.13 | 1.39 |
| ISGF3-γ | AFFX- | — | 4.4 +/− 0.08 | 3.6 +/− 0.10 | 2.8 +/− 0.15 | 1.57 | 1.23 | 1.27 |
| | HUMISGF3A/M97935_{—} | | | | | | | |
| | 3_at | | | | | | | |
| C11orf25 | 215241_at | — | 1.5 +/− 0.07 | 1.2 +/− 0.12 | 0.9 +/− 0.03 | 1.56 | 1.19 | 1.31 |
| SOD2 | 216841_s_at | 147460 | 1.5 +/− 0.18 | 1.6 +/− 0.12 | 1.0 +/− 0.11 | 1.55 | 0.93 | 1.66 |
| DNPEP | 38703_at | — | 1.6 +/− 0.08 | 1.5 +/− 0.08 | 1.0 +/− 0.08 | 1.52 | 1.04 | 1.46 |
| FLJ20637 | 219352_at | — | 11.7 +/− 1.81 | 3.7 +/− 0.77 | 7.8 +/− 1.83 | 1.51 | 3.15 | 0.48 |
| SOD2 | 215223_s_at | 147460 | 2.1 +/− 0.12 | 2.1 +/− 0.35 | 1.4 +/− 0.11 | 1.50 | 1.01 | 1.48 |
| TNFSF10 | 202688_at | 603598 | 1.8 +/− 0.06 | 1.7 +/− 0.19 | 1.2 +/− 0.08 | 1.50 | 1.09 | 1.38 |
| TNFSF10 | 202687_s_at | 603598 | 1.9 +/− 0.17 | 1.6 +/− 0.10 | 1.2 +/− 0.13 | 1.49 | 1.15 | 1.29 |
| MT2A | 212859_x_at | 156360 | 1.8 +/− 0.03 | 1.3 +/− 0.09 | 1.2 +/− 0.09 | 1.47 | 1.34 | 1.09 |
| OAS3 | 218400_at | 603351 | 4.9 +/− 0.15 | 1.6 +/− 0.09 | 3.4 +/− 0.27 | 1.44 | 3.11 | 0.46 |
| TGM2 | 211003_x_at | 190196 | 1.8 +/− 0.11 | 1.8 +/− 0.22 | 1.3 +/− 0.27 | 1.44 | 0.98 | 1.46 |
| MT1X | 204326_x_at | 156359 | 1.7 +/− 0.07 | 1.3 +/− 0.07 | 1.2 +/− 0.02 | 1.44 | 1.31 | 1.10 |
| OSMR | 205729_at | 601743 | 1.5 +/− 0.05 | 1.3 +/− 0.18 | 1.1 +/− 0.14 | 1.42 | 1.21 | 1.18 |
| TRIM31 | 215444_s_at | — | 1.5 +/− 0.08 | 1.7 +/− 0.17 | 1.1 +/− 0.09 | 1.42 | 0.90 | 1.57 |
| ANGPTL6 | 53720_at | — | 2.5 +/− 0.27 | 2.0 +/− 0.28 | 1.8 +/− 0.30 | 1.39 | 1.26 | 1.10 |
| SERPINE1 | 202628_s_at | 173360 | 1.7 +/− 0.09 | 1.0 +/− 0.51 | 1.2 +/− 0.59 | 1.38 | 1.67 | 0.83 |
| RI58 | 203595_s_at | — | 3.3 +/− 0.19 | 1.6 +/− 0.19 | 2.4 +/− 0.55 | 1.35 | 2.00 | 0.67 |
| B2M | 216231_s_at | 109700 | 1.8 +/− 0.05 | 1.6 +/− 0.13 | 1.3 +/− 0.03 | 1.33 | 1.13 | 1.18 |
| TCIRG1 | 204158_s_at | 604592 | 1.6 +/− 0.07 | 1.4 +/− 0.14 | 1.2 +/− 0.05 | 1.33 | 1.18 | 1.13 |
| MDA5 | 219209_at | 606951 | 14.1 +/− 2.15 | 4.0 +/− 1.21 | 10.7 +/− 1.29 | 1.33 | 3.51 | 0.38 |
| STAT1 | 200887_s_at | 600555 | 3.9 +/− 0.24 | 3.5 +/− 0.27 | 2.9 +/− 0.49 | 1.32 | 1.13 | 1.17 |
| ANXA1 | 201012_at | 151690 | 1.7 +/− 0.07 | 1.0 +/− 0.18 | 1.3 +/− 0.10 | 1.31 | 1.66 | 0.79 |
| SERPINE1 | 202627_s_at | 173360 | 1.8 +/− 0.04 | 1.2 +/− 0.03 | 1.4 +/− 0.09 | 1.30 | 1.46 | 0.89 |
| SAMHD1 | 204502_at | 606754 | 3.0 +/− 0.44 | 2.1 +/− 0.29 | 2.3 +/− 0.53 | 1.30 | 1.43 | 0.91 |
| FLJ22693 | 218543_s_at | — | 2.3 +/− 0.06 | 1.5 +/− 0.04 | 1.8 +/− 0.10 | 1.29 | 1.47 | 0.88 |
| LAP3 | 217933_s_at | 170250 | 2.1 +/− 0.04 | 1.5 +/− 0.06 | 1.7 +/− 0.07 | 1.28 | 1.46 | 0.88 |
| CDC25B | 201853_s_at | 116949 | 1.3 +/− 0.01 | 1.4 +/− 0.06 | 1.0 +/− 0.07 | 1.28 | 0.97 | 1.33 |
| ZNF337 | 37860_at | — | 0.9 +/− 0.12 | 0.8 +/− 0.13 | 0.7 +/− 0.02 | 1.28 | 1.07 | 1.20 |
| RCOR | 212612_at | 607675 | 1.0 +/− 0.04 | 0.9 +/− 0.11 | 0.8 +/− 0.01 | 1.27 | 1.05 | 1.21 |
| HNOEL-iso | 218162_at | — | 1.5 +/− 0.04 | 1.2 +/− 0.12 | 1.2 +/− 0.09 | 1.27 | 1.18 | 1.08 |
| PSME1 | 200814_at | 600654 | 1.8 +/− 0.05 | 1.5 +/− 0.09 | 1.4 +/− 0.24 | 1.26 | 1.14 | 1.10 |
| TRIM31 | 208170_s_at | — | 1.5 +/− 0.08 | 1.5 +/− 0.05 | 1.2 +/− 0.02 | 1.25 | 0.99 | 1.27 |
| LEPROTL1 | 202594_at | 607338 | 1.2 +/− 0.03 | 1.2 +/− 0.04 | 1.0 +/− 0.01 | 1.25 | 1.01 | 1.24 |
| ISGF3G | 203882_at | 147574 | 14.1 +/− 1.21 | 7.2 +/− 0.14 | 11.4 +/− 0.62 | 1.24 | 1.95 | 0.64 |
| NBS1 | 202907_s_at | 602667 | 1.2 +/− 0.01 | 1.2 +/− 0.18 | 1.0 +/− 0.01 | 1.23 | 1.03 | 1.19 |
| PLOD2 | 202620_s_at | 601865 | 1.3 +/− 0.02 | 1.2 +/− 0.09 | 1.1 +/− 0.06 | 1.23 | 1.12 | 1.09 |
| HPGD | 203914_x_at | 601688 | 1.5 +/− 0.09 | 1.3 +/− 0.05 | 1.2 +/− 0.10 | 1.21 | 1.17 | 1.03 |
| B2M | 201891_s_at | 109700 | 1.8 +/− 0.09 | 1.7 +/− 0.19 | 1.5 +/− 0.14 | 1.20 | 1.06 | 1.13 |
| HRASLS3 | 209581_at | — | 1.3 +/− 0.04 | 1.3 +/− 0.07 | 1.1 +/− 0.02 | 1.20 | 0.99 | 1.21 |
| HMGCR | 202540_s_at | 142910 | 1.1 +/− 0.05 | 1.0 +/− 0.06 | 0.9 +/− 0.01 | 1.20 | 1.08 | 1.11 |
| PSMA3 | 201532_at | 176843 | 1.2 +/− 0.05 | 1.2 +/− 0.03 | 1.0 +/− 0.01 | 1.19 | 1.01 | 1.18 |
| TSC | 218872_at | — | 0.9 +/− 0.09 | 0.7 +/− 0.02 | 0.8 +/− 0.28 | 1.18 | 1.31 | 0.90 |
| OAS1 | 205552_s_at | 164350 | 7.2 +/− 0.20 | 1.7 +/− 0.28 | 6.1 +/− 1.08 | 1.17 | 4.29 | 0.27 |
| PLOD2 | 202619_s_at | 601865 | 1.3 +/− 0.02 | 1.2 +/− 0.05 | 1.2 +/− 0.10 | 1.16 | 1.13 | 1.02 |
| CGI-109 | 209404_s_at | — | 1.0 +/− 0.02 | 1.0 +/− 0.03 | 0.9 +/− 0.01 | 1.16 | 1.07 | 1.08 |
| TDO2 | 205943_at | 191070 | 1.6 +/− 0.08 | 0.8 +/− 0.06 | 1.4 +/− 0.15 | 1.15 | 2.07 | 0.56 |
| USP18 | 219211_at | 607057 | 4.6 +/− 0.40 | 1.6 +/− 0.24 | 4.0 +/− 0.40 | 1.15 | 2.84 | 0.40 |
| UCP2 | 208998_at | 601693 | 1.4 +/− 0.05 | 1.4 +/− 0.06 | 1.2 +/− 0.03 | 1.14 | 0.95 | 1.20 |
| RIG-I | 218943_s_at | — | 5.2 +/− 0.52 | 2.0 +/− 0.40 | 4.6 +/− 0.27 | 1.13 | 2.59 | 0.44 |
| CEACAM1 | 206576_s_at | 109770 | 1.4 +/− 0.07 | 1.5 +/− 0.05 | 1.3 +/− 0.08 | 1.12 | 0.96 | 1.17 |
| CEACAM1 | 209498_at | 109770 | 1.4 +/− 0.04 | 1.4 +/− 0.04 | 1.3 +/− 0.09 | 1.12 | 1.04 | 1.08 |
| EIF4A2 | 200912_s_at | 601102 | 1.1 +/− 0.02 | 1.0 +/− 0.02 | 1.0 +/− 0.01 | 1.11 | 1.10 | 1.01 |
| TRIM14 | 210846_x_at | 606556 | 1.7 +/− 0.05 | 1.4 +/− 0.34 | 1.5 +/− 0.30 | 1.11 | 1.17 | 0.95 |
| FACL3 | 201661_s_at | 602371 | 1.2 +/− 0.02 | 1.3 +/− 0.03 | 1.1 +/− 0.11 | 1.10 | 0.94 | 1.16 |
| CKB | 200884_at | 123280 | 1.2 +/− 0.02 | 1.1 +/− 0.06 | 1.1 +/− 0.01 | 1.09 | 1.10 | 0.99 |
| KPNA2 | 201088_at | 600685 | 0.9 +/− 0.06 | 0.9 +/− 0.04 | 0.8 +/− 0.01 | 1.09 | 0.99 | 1.10 |
| LGALS8 | 208934_s_at | 606099 | 1.2 +/− 0.01 | 1.2 +/− 0.01 | 1.1 +/− 0.03 | 1.09 | 1.02 | 1.07 |
| TRIM14 | 203148_s_at | 606556 | 2.4 +/− 0.21 | 1.7 +/− 0.14 | 2.2 +/− 0.24 | 1.09 | 1.38 | 0.79 |
| SP110 | 209762_x_at | 604457 | 2.7 +/− 0.11 | 1.5 +/− 0.19 | 2.5 +/− 0.23 | 1.08 | 1.81 | 0.60 |
| SP110 | 208012_x_at | 604457 | 2.8 +/− 0.15 | 1.5 +/− 0.08 | 2.6 +/− 0.07 | 1.08 | 1.90 | 0.57 |
| TXNL | 201588_at | 603049 | 1.1 +/− 0.05 | 1.1 +/− 0.01 | 1.0 +/− 0.04 | 1.06 | 0.96 | 1.11 |
| STAT3 | 208991_at | 102582 | 1.3 +/− 0.03 | 1.1 +/− 0.07 | 1.2 +/− 0.06 | 1.05 | 1.15 | 0.91 |
| STAT6 | 201331_s_at | 601512 | 1.3 +/− 0.11 | 1.2 +/− 0.07 | 1.2 +/− 0.02 | 1.05 | 1.10 | 0.95 |
| SFN | 33322_i_at | 601290 | 1.3 +/− 0.05 | 1.1 +/− 0.04 | 1.3 +/− 0.08 | 1.04 | 1.19 | 0.87 |
| BTF | 214499_s_at | — | 0.9 +/− 0.05 | 1.1 +/− 0.04 | 0.9 +/− 0.02 | 1.04 | 0.79 | 1.31 |
| NDUFA9 | 208969_at | 603834 | 1.1 +/− 0.03 | 1.2 +/− 0.01 | 1.0 +/− 0.02 | 1.03 | 0.93 | 1.11 |
| — | 215071_s_at | — | 1.1 +/− 0.03 | 0.9 +/− 0.01 | 1.1 +/− 0.04 | 1.03 | 1.22 | 0.84 |
| ADAR | 201786_s_at | 601059 | 1.6 +/− 0.06 | 1.3 +/− 0.03 | 1.5 +/− 0.12 | 1.03 | 1.26 | 0.81 |
| NFIL3 | 203574_at | 605327 | 1.1 +/− 0.04 | 1.1 +/− 0.05 | 1.1 +/− 0.01 | 1.02 | 1.03 | 0.99 |
| ORMDL2 | 218556_at | — | 1.2 +/− 0.03 | 1.0 +/− 0.02 | 1.2 +/− 0.02 | 1.02 | 1.17 | 0.87 |
| OAZIN | 212461_at | 607909 | 0.9 +/− 0.02 | 0.9 +/− 0.03 | 0.9 +/− 0.01 | 1.02 | 0.92 | 1.11 |
| PCTAIRE2BP | 213361_at | — | 1.5 +/− 0.01 | 1.1 +/− 0.08 | 1.5 +/− 0.07 | 1.00 | 1.35 | 0.74 |
| PGLS | 218388_at | 604951 | 1.2 +/− 0.09 | 1.0 +/− 0.04 | 1.1 +/− 0.01 | 1.00 | 1.12 | 0.90 |
| CCNB1 | 214710_s_at | 123836 | 0.9 +/− 0.03 | 1.0 +/− 0.03 | 0.9 +/− 0.00 | 1.00 | 0.91 | 1.09 |
| H1FX | 204805_s_at | 602785 | 1.2 +/− 0.05 | 0.9 +/− 0.05 | 1.2 +/− 0.03 | 0.98 | 1.33 | 0.74 |
| PLSCR1 | 202446_s_at | 604170 | 4.0 +/− 0.24 | 1.1 +/− 0.07 | 4.0 +/− 0.28 | 0.98 | 3.47 | 0.28 |
| DLG1 | 217208_s_at | 601014 | 0.7 +/− 0.05 | 0.9 +/− 0.06 | 0.8 +/− 0.03 | 0.98 | 0.87 | 1.12 |
| AGR2 | 209173_at | 606358 | 1.2 +/− 0.07 | 0.8 +/− 0.01 | 1.2 +/− 0.07 | 0.97 | 1.45 | 0.67 |
| PTTG1IP | 200677_at | 603784 | 1.1 +/− 0.05 | 1.0 +/− 0.01 | 1.1 +/− 0.01 | 0.97 | 1.02 | 0.95 |
| PROM1 | 204304_s_at | 604365 | 1.0 +/− 0.02 | 0.8 +/− 0.01 | 1.0 +/− 0.06 | 0.96 | 1.16 | 0.83 |
| — | 200776_s_at | — | 1.0 +/− 0.05 | 1.2 +/− 0.03 | 1.0 +/− 0.04 | 0.96 | 0.81 | 1.19 |
| BCL3 | 204908_s_at | 109560 | 1.3 +/− 0.11 | 1.5 +/− 0.08 | 1.4 +/− 0.15 | 0.95 | 0.89 | 1.08 |
| G1P3 | 204415_at | 147572 | 26.4 +/− 2.28 | 4.3 +/− 0.52 | 27.7 +/− 2.83 | 0.95 | 6.09 | 0.16 |
| G1P2 | 205483_s_at | 147571 | 21.2 +/− 0.83 | 3.3 +/− 0.13 | 22.3 +/− 0.76 | 0.95 | 6.45 | 0.15 |
| ACVR1B | 213198_at | 601300 | 1.0 +/− 0.06 | 1.0 +/− 0.02 | 1.1 +/− 0.01 | 0.95 | 1.03 | 0.92 |
| ANKT | 218039_at | — | 0.9 +/− 0.01 | 1.0 +/− 0.02 | 1.0 +/− 0.01 | 0.95 | 0.95 | 1.00 |
| OAS1 | 202869_at | 164350 | 5.2 +/− 0.36 | 1.5 +/− 0.25 | 5.5 +/− 0.59 | 0.94 | 3.43 | 0.27 |
| FLJ38348 | 213294_at | — | 2.3 +/− 0.04 | 1.1 +/− 0.08 | 2.5 +/− 0.33 | 0.94 | 2.06 | 0.45 |
| LIPA | 201847_at | 278000 | 1.1 +/− 0.05 | 1.0 +/− 0.05 | 1.2 +/− 0.02 | 0.93 | 1.13 | 0.83 |
| PRKR | 204211_x_at | 176871 | 2.6 +/− 0.15 | 1.5 +/− 0.16 | 2.8 +/− 0.17 | 0.93 | 1.77 | 0.53 |
| TGFBR2 | 208944_at | 190182 | 1.2 +/− 0.02 | 1.0 +/− 0.07 | 1.3 +/− 0.06 | 0.93 | 1.14 | 0.81 |
| TTC3 | 210645_s_at | 602259 | 0.8 +/− 0.05 | 0.8 +/− 0.01 | 0.8 +/− 0.05 | 0.93 | 0.98 | 0.94 |
| MARCKS | 201670_s_at | 177061 | 1.0 +/− 0.01 | 1.1 +/− 0.03 | 1.1 +/− 0.00 | 0.92 | 0.93 | 0.99 |
| ABLIM1 | 200965_s_at | 602330 | 0.8 +/− 0.02 | 0.9 +/− 0.02 | 0.9 +/− 0.02 | 0.91 | 0.91 | 1.00 |
| STAU | 213037_x_at | 601716 | 1.0 +/− 0.07 | 1.1 +/− 0.05 | 1.1 +/− 0.02 | 0.90 | 0.94 | 0.96 |
| SORD | 201563_at | 182500 | 0.9 +/− 0.02 | 1.0 +/− 0.01 | 1.0 +/− 0.02 | 0.90 | 0.87 | 1.03 |
| ZDHHC4 | 220261_s_at | — | 0.9 +/− 0.01 | 0.9 +/− 0.06 | 1.0 +/− 0.01 | 0.89 | 0.93 | 0.96 |
| PLSCR1 | 202430_s_at | 604170 | 3.9 +/− 0.12 | 1.6 +/− 0.43 | 4.4 +/− 0.27 | 0.89 | 2.42 | 0.37 |
| MX1 | 202086_at | 147150 | 20.4 +/− 0.69 | 1.7 +/− 0.16 | 23.0 +/− 2.34 | 0.89 | 11.82 | 0.08 |
| HRBL | 206821_x_at | 604019 | 1.1 +/− 0.09 | 1.3 +/− 0.03 | 1.2 +/− 0.08 | 0.88 | 0.85 | 1.04 |
| CSPG2 | 221731_x_at | 118661 | 0.9 +/− 0.03 | 0.8 +/− 0.03 | 1.1 +/− 0.04 | 0.88 | 1.25 | 0.71 |
| NTS | 206291_at | 162650 | 1.0 +/− 0.06 | 0.9 +/− 0.03 | 1.2 +/− 0.01 | 0.86 | 1.07 | 0.81 |
| IFIT1 | 203153_at | 147690 | 55.0 +/− 10.34 | 2.1 +/− 0.68 | 63.9 +/− 6.29 | 0.86 | 26.41 | 0.03 |
| GYG2 | 215695_s_at | 300198 | 0.7 +/− 0.04 | 0.9 +/− 0.03 | 0.8 +/− 0.04 | 0.84 | 0.77 | 1.10 |
| KNG | 206054_at | 228960 | 0.7 +/− 0.04 | 0.8 +/− 0.07 | 0.8 +/− 0.07 | 0.83 | 0.84 | 0.99 |
| GBA3 | 219954_s_at | 606619 | 0.8 +/− 0.02 | 0.7 +/− 0.02 | 1.0 +/− 0.05 | 0.82 | 1.10 | 0.75 |
| NMT1 | 201158_at | 160993 | 1.1 +/− 0.18 | 1.2 +/− 0.02 | 1.3 +/− 0.00 | 0.82 | 0.93 | 0.89 |
| SOAT2 | 210677_at | 601311 | 1.1 +/− 0.08 | 0.9 +/− 0.02 | 1.3 +/− 0.03 | 0.81 | 1.13 | 0.71 |
| NFKBIA | 201502_s_at | 164008 | 1.2 +/− 0.03 | 1.3 +/− 0.07 | 1.5 +/− 0.05 | 0.80 | 0.96 | 0.83 |
| TNFSF9 | 206907_at | 606182 | 1.1 +/− 0.05 | 1.1 +/− 0.11 | 1.4 +/− 0.03 | 0.80 | 1.04 | 0.76 |
| FLJ12442 | 218051_s_at | — | 0.7 +/− 0.03 | 0.9 +/− 0.06 | 0.9 +/− 0.18 | 0.78 | 0.80 | 0.97 |
| BEX1 | 218332_at | — | 0.8 +/− 0.01 | 0.8 +/− 0.07 | 1.1 +/− 0.03 | 0.76 | 1.01 | 0.75 |
| NDRG4 | 209159_s_at | — | 0.8 +/− 0.02 | 0.8 +/− 0.09 | 1.2 +/− 0.07 | 0.62 | 0.97 | 0.64 |
| FLJ10140 | 213634_s_at | — | 0.7 +/− 0.02 | 1.1 +/− 0.08 | 1.2 +/− 0.05 | 0.60 | 0.65 | 0.93 |

¹Expression level as ratio of signal following IFN treatment relative to untreated cells following dosing at human Cₘₐₓ (IFN alfacon-1 = 80 pg/mL, IFN γ-1b = 300 pg/mL)
²Ratio of expression change in the two listed treatments as a percent
³Affymetrix U133A microarray probe set ID
⁴Online Mendelian Inheritance in Man reference number; www.ncbi.nlm.nih.gov/entrez/query.fcgi?CMD=search&DB=omim
⁵IFN alfacon-1 plus IFN γ-1b

Table 7 shows the effect of exposure to an EC 50 level of interferon alphacon or or interferon-gamma 1b or the combination of interferon alphacon and interferon-gamma 1b on the expression ratios of several antiviral genes in A-549 cells. Gene expression of the antiviral gene set is increased upon exposure to the interferon alphacon 1 and interferon-gamma 1b combination as compared to exposure to either interferon alphacon 1 or to interferon-gamma 1b alone.

TABLE 7
Genes showing statistically significant differences in expression in A549 cells following EC₅₀ exposure
to IFN alfacon-1, IFN γ-1b, or both

| | | | Expression Level (treated/untreated)¹ | | | | |
|---|---|---|---|---|---|---|---|
| | | | IFN γ-1b (pg/mL) | | | | |
| | | | 65.1 | 291.5 | | 65.1 | |
| | | | IFN alfacon-1 (pg/mL) | | | | |
| Gene | OMIM² | Probe³ | 1.1 | | 5.3 | | 1.1 |
| AACS | — | 218434_s_at | 0.7 +/− 0.05 | 0.6 +/− 0.06 | 1.0 +/− 0.15 | 0.8 +/− 0.10 | 0.9 +/− 0.04 |
| ABCC1 | 158343 | 202804_at | 1.1 +/− 0.10 | 1.3 +/− 0.05 | 1.0 +/− 0.09 | 1.2 +/− 0.10 | 1.0 +/− 0.04 |
| ABCG1 | 603076 | 204567_s_at | 0.7 +/− 0.17 | 0.5 +/− 0.09 | 1.0 +/− 0.13 | 0.8 +/− 0.08 | 1.0 +/− 0.14 |
| ABR | 600365 | 212895_s_at | 0.8 +/− 0.07 | 0.7 +/− 0.05 | 1.0 +/− 0.08 | 0.8 +/− 0.08 | 0.9 +/− 0.07 |
| ACAD8 | 604773 | 221669_s_at | 0.8 +/− 0.06 | 0.7 +/− 0.05 | 1.0 +/− 0.10 | 0.8 +/− 0.11 | 0.9 +/− 0.05 |
| ACATE2 | — | 221641_s_at | 0.9 +/− 0.10 | 0.9 +/− 0.08 | 1.0 +/− 0.14 | 0.7 +/− 0.05 | 1.0 +/− 0.07 |
| ACY-3 | — | 230481_at | 2.6 +/− 0.21 | 3.8 +/− 0.56 | 1.4 +/− 0.49 | 3.3 +/− 0.88 | 1.2 +/− 0.29 |
| ADAR | 601059 | 201786_s_at | 1.5 +/− 0.15 | 1.7 +/− 0.10 | 1.5 +/− 0.11 | 1.3 +/− 0.10 | 1.2 +/− 0.03 |
| ADPRHL1 | — | 1553163_at | 1.0 +/− 0.09 | 1.0 +/− 0.30 | 1.1 +/− 0.29 | 1.6 +/− 0.26 | 1.7 +/− 0.25 |
| ADPRTL3 | 607726 | 209940_at | 1.2 +/− 0.12 | 1.6 +/− 0.21 | 0.9 +/− 0.13 | 1.3 +/− 0.14 | 0.9 +/− 0.07 |
| AGPAT3 | — | 223184_s_at | 1.1 +/− 0.08 | 1.2 +/− 0.05 | 0.9 +/− 0.07 | 1.0 +/− 0.08 | 1.0 +/− 0.08 |
| AGRN | 103320 | 217419_x_at | 1.0 +/− 0.09 | 0.8 +/− 0.09 | 1.1 +/− 0.05 | 0.8 +/− 0.07 | 1.0 +/− 0.10 |
| AKAP12 | 604698 | 227529_s_at | 1.1 +/− 0.13 | 1.4 +/− 0.08 | 1.0 +/− 0.15 | 1.1 +/− 0.08 | 1.0 +/− 0.05 |
| AKAP12 | 604698 | 227530_at | 1.1 +/− 0.16 | 1.4 +/− 0.06 | 1.0 +/− 0.09 | 1.2 +/− 0.14 | 1.0 +/− 0.11 |
| AKAP12 | 604698 | 231067_s_at | 1.3 +/− 0.03 | 1.6 +/− 0.13 | 1.0 +/− 0.08 | 1.2 +/− 0.18 | 1.1 +/− 0.05 |
| ALEX3 | 300364 | 222444_at | 0.9 +/− 0.01 | 0.7 +/− 0.04 | 1.0 +/− 0.14 | 0.8 +/− 0.07 | 0.9 +/− 0.07 |
| ALS2CR9 | — | 1552482_at | 1.1 +/− 0.12 | 0.6 +/− 0.12 | 1.0 +/− 0.12 | 0.7 +/− 0.07 | 1.1 +/− 0.23 |
| ANGPTL6 | — | 53720_at | 1.6 +/− 0.11 | 2.0 +/− 0.30 | 1.7 +/− 0.22 | 1.5 +/− 0.18 | 1.2 +/− 0.04 |
| APOL1 | 603743 | 209546_s_at | 6.8 +/− 0.50 | 12.7 +/− 0.55 | 1.1 +/− 0.32 | 7.2 +/− 0.40 | 1.2 +/− 0.28 |
| APOL2 | 607252 | 221653_x_at | 4.6 +/− 1.18 | 10.2 +/− 1.02 | 2.1 +/− 0.60 | 3.8 +/− 0.77 | 1.6 +/− 0.43 |
| APOL3 | 607253 | 221087_s_at | 7.5 +/− 1.11 | 18.6 +/− 1.97 | 1.1 +/− 0.84 | 8.7 +/− 1.53 | 0.9 +/− 0.39 |
| APOL6 | 607256 | 219716_at | 3.8 +/− 1.07 | 7.6 +/− 1.00 | 1.4 +/− 0.42 | 4.2 +/− 0.62 | 1.3 +/− 0.22 |
| APOL6 | 607256 | 241869_at | 4.2 +/− 0.27 | 6.2 +/− 0.66 | 1.5 +/− 0.25 | 4.4 +/− 0.61 | 1.2 +/− 0.18 |
| AQP3 | 600170 | 39248_at | 0.7 +/− 0.12 | 0.5 +/− 0.08 | 1.0 +/− 0.16 | 0.7 +/− 0.12 | 0.9 +/− 0.10 |
| ARK5 | 608130 | 204589_at | 0.8 +/− 0.10 | 0.7 +/− 0.14 | 1.1 +/− 0.10 | 0.8 +/− 0.10 | 1.0 +/− 0.15 |
| ARNTL2 | — | 223586_at | 1.2 +/− 0.24 | 1.5 +/− 0.16 | 0.9 +/− 0.24 | 1.2 +/− 0.11 | 0.8 +/− 0.15 |
| ARTS-1 | 606832 | 209788_s_at | 2.0 +/− 0.22 | 2.4 +/− 0.45 | 1.4 +/− 0.34 | 2.1 +/− 0.14 | 1.1 +/− 0.48 |
| ARTS-1 | 606832 | 210385_s_at | 1.8 +/− 0.28 | 2.2 +/− 0.19 | 1.3 +/− 0.28 | 2.0 +/− 0.26 | 1.3 +/− 0.26 |
| ARTS-1 | 606832 | 214012_at | 3.9 +/− 1.18 | 5.2 +/− 0.82 | 1.5 +/− 0.28 | 4.8 +/− 0.71 | 1.2 +/− 0.29 |
| ASB3 | 605760 | 224524_s_at | 1.0 +/− 0.03 | 1.0 +/− 0.04 | 1.1 +/− 0.14 | 1.0 +/− 0.07 | 1.2 +/− 0.09 |
| ASF1B | — | 218115_at | 0.9 +/− 0.05 | 0.8 +/− 0.05 | 1.0 +/− 0.07 | 0.9 +/− 0.09 | 1.0 +/− 0.02 |
| ASPM | 605481 | 219918_s_at | 1.2 +/− 0.11 | 1.3 +/− 0.14 | 1.0 +/− 0.08 | 1.1 +/− 0.10 | 1.0 +/− 0.09 |
| ATF3 | 603148 | 202672_s_at | 1.1 +/− 0.19 | 1.6 +/− 0.07 | 1.1 +/− 0.03 | 1.1 +/− 0.12 | 1.1 +/− 0.16 |
| ATP10D | — | 213238_at | 1.5 +/− 0.09 | 2.3 +/− 0.17 | 1.0 +/− 0.22 | 1.6 +/− 0.23 | 1.0 +/− 0.09 |
| ATP6V1C1 | 603097 | 226463_at | 1.0 +/− 0.06 | 1.0 +/− 0.04 | 0.9 +/− 0.06 | 1.1 +/− 0.05 | 0.9 +/− 0.09 |
| ATP8B1 | 602397 | 226302_at | 0.7 +/− 0.08 | 0.6 +/− 0.02 | 0.9 +/− 0.16 | 0.8 +/− 0.04 | 0.9 +/− 0.12 |
| AZ2 | — | 227904_at | 0.8 +/− 0.09 | 0.8 +/− 0.05 | 1.0 +/− 0.03 | 0.9 +/− 0.10 | 1.0 +/− 0.04 |
| B2M | 109700 | 201891_s_at | 1.6 +/− 0.14 | 1.7 +/− 0.15 | 1.2 +/− 0.06 | 1.4 +/− 0.08 | 1.2 +/− 0.08 |
| B2M | 109700 | 216231_s_at | 1.8 +/− 0.11 | 2.0 +/− 0.18 | 1.3 +/− 0.16 | 1.7 +/− 0.13 | 1.2 +/− 0.11 |
| B4GALT1 | 137060 | 238987_at | 1.2 +/− 0.14 | 1.4 +/− 0.24 | 0.9 +/− 0.08 | 1.3 +/− 0.07 | 1.0 +/− 0.03 |
| BAD | 603167 | 1861_at | 0.9 +/− 0.07 | 0.7 +/− 0.10 | 0.9 +/− 0.07 | 0.8 +/− 0.03 | 1.0 +/− 0.07 |
| BAD | 603167 | 209364_at | 0.8 +/− 0.14 | 0.7 +/− 0.08 | 1.0 +/− 0.08 | 0.7 +/− 0.03 | 1.0 +/− 0.07 |
| BAL | — | 223220_s_at | 5.0 +/− 0.26 | 7.2 +/− 0.26 | 2.9 +/− 0.26 | 3.8 +/− 0.29 | 1.8 +/− 0.08 |
| BAL | — | 227807_at | 2.2 +/− 0.13 | 2.8 +/− 0.25 | 1.7 +/− 0.20 | 1.8 +/− 0.16 | 1.5 +/− 0.21 |
| BANK | — | 1558662_s_at | 1.4 +/− 0.14 | 1.5 +/− 0.27 | 1.0 +/− 0.18 | 1.5 +/− 0.19 | 0.9 +/− 0.15 |
| BAT5 | 142620 | 224756_s_at | 1.3 +/− 0.14 | 1.4 +/− 0.02 | 1.1 +/− 0.07 | 1.3 +/− 0.10 | 1.0 +/− 0.13 |
| BCDO1 | 605748 | 220087_at | 1.7 +/− 0.23 | 2.2 +/− 0.12 | 1.0 +/− 0.19 | 1.7 +/− 0.24 | 1.2 +/− 0.27 |
| BENE | 602222 | 209373_at | 0.8 +/− 0.10 | 0.6 +/− 0.08 | 1.0 +/− 0.10 | 0.8 +/− 0.14 | 1.0 +/− 0.09 |
| BRI3BP | — | 231810_at | 0.9 +/− 0.04 | 0.9 +/− 0.03 | 1.0 +/− 0.04 | 0.9 +/− 0.02 | 0.9 +/− 0.02 |
| BST2 | 600534 | 201641_at | 47.8 +/− 8.25 | 71.6 +/− 9.40 | 11.5 +/− 5.85 | 24.2 +/− 9.26 | 3.7 +/− 2.12 |
| BTN3A1 | — | 209770_at | 1.3 +/− 0.14 | 1.8 +/− 0.21 | 1.0 +/− 0.15 | 1.2 +/− 0.24 | 1.2 +/− 0.31 |
| BTN3A2 | — | 209846_s_at | 2.7 +/− 0.49 | 3.2 +/− 0.11 | 1.2 +/− 0.10 | 2.2 +/− 0.18 | 1.0 +/− 0.22 |
| BTN3A2 | — | 212613_at | 3.0 +/− 0.43 | 3.2 +/− 0.67 | 0.6 +/− 0.20 | 3.0 +/− 0.58 | 0.7 +/− 0.43 |
| BTN3A3 | — | 204820_s_at | 2.6 +/− 0.22 | 3.0 +/− 0.10 | 1.3 +/− 0.11 | 2.6 +/− 0.19 | 1.1 +/− 0.31 |
| BTN3A3 | — | 204821_at | 4.0 +/− 0.94 | 6.5 +/− 1.11 | 1.1 +/− 0.56 | 5.0 +/− 0.86 | 1.1 +/− 0.42 |
| BTN3A3 | — | 38241_at | 4.6 +/− 1.25 | 8.7 +/− 1.00 | 1.1 +/− 0.32 | 5.7 +/− 1.06 | 0.8 +/− 0.30 |
| C13orf11 | — | 226050_at | 0.7 +/− 0.07 | 0.6 +/− 0.07 | 0.9 +/− 0.11 | 0.8 +/− 0.07 | 1.0 +/− 0.10 |
| C13orf12 | — | 217769_s_at | 1.1 +/− 0.05 | 1.2 +/− 0.07 | 1.0 +/− 0.07 | 1.0 +/− 0.05 | 1.0 +/− 0.06 |
| C14orf1 | 604576 | 202562_s_at | 0.9 +/− 0.07 | 0.7 +/− 0.05 | 1.0 +/− 0.05 | 0.9 +/− 0.03 | 1.0 +/− 0.02 |
| C14orf122 | — | 219203_at | 1.0 +/− 0.06 | 0.8 +/− 0.04 | 1.1 +/− 0.12 | 0.9 +/− 0.04 | 1.1 +/− 0.03 |
| C14orf147 | — | 212460_at | 1.2 +/− 0.13 | 1.4 +/− 0.03 | 1.0 +/− 0.08 | 1.2 +/− 0.15 | 1.1 +/− 0.08 |
| C14orf159 | — | 218298_s_at | 1.6 +/− 0.22 | 1.7 +/− 0.16 | 1.1 +/− 0.17 | 1.5 +/− 0.26 | 1.1 +/− 0.11 |
| C14orf31 | — | 225464_at | 0.8 +/− 0.10 | 0.6 +/− 0.06 | 0.9 +/− 0.09 | 0.7 +/− 0.06 | 1.0 +/− 0.07 |
| C1orf29 | — | 204439_at | 13.4 +/− 1.64 | 13.0 +/− 2.72 | 15.7 +/− 3.34 | 2.9 +/− 1.76 | 2.1 +/− 0.98 |
| C1RL | — | 218983_at | 1.2 +/− 0.06 | 1.4 +/− 0.09 | 0.9 +/− 0.08 | 1.3 +/− 0.11 | 1.0 +/− 0.10 |
| C1S | 120580 | 1555229_a_at | 11.2 +/− 4.20 | 14.5 +/− 5.64 | 1.9 +/− 0.57 | 10.8 +/− 6.54 | 1.4 +/− 0.22 |
| C1S | 120580 | 208747_s_at | 9.5 +/− 1.20 | 14.0 +/− 0.74 | 1.4 +/− 0.38 | 10.0 +/− 1.05 | 1.0 +/− 0.34 |
| C20orf139 | — | 225252_at | 1.0 +/− 0.04 | 1.2 +/− 0.05 | 1.0 +/− 0.02 | 1.0 +/− 0.07 | 1.0 +/− 0.06 |
| C20orf18 | — | 207713_s_at | 1.3 +/− 0.12 | 1.5 +/− 0.16 | 1.0 +/− 0.19 | 1.3 +/− 0.12 | 0.9 +/− 0.17 |
| C20orf18 | — | 221827_at | 1.3 +/− 0.04 | 1.6 +/− 0.06 | 1.1 +/− 0.07 | 1.2 +/− 0.05 | 1.1 +/− 0.05 |
| C21orf63 | — | 227188_at | 1.2 +/− 0.05 | 1.4 +/− 0.08 | 1.0 +/− 0.09 | 1.2 +/− 0.06 | 1.0 +/− 0.06 |
| C22orf2 | 607757 | 203450_at | 0.8 +/− 0.03 | 0.7 +/− 0.09 | 1.0 +/− 0.07 | 0.8 +/− 0.10 | 1.0 +/− 0.11 |
| C3 | 120700 | 217767_at | 1.5 +/− 0.27 | 2.1 +/− 0.31 | 1.0 +/− 0.14 | 1.5 +/− 0.38 | 0.9 +/− 0.07 |
| C3orf8 | — | 201906_s_at | 0.7 +/− 0.04 | 0.6 +/− 0.03 | 0.8 +/− 0.10 | 0.8 +/− 0.03 | 0.8 +/− 0.06 |
| C7orf32 | — | 213587_s_at | 0.8 +/− 0.01 | 0.7 +/− 0.01 | 0.9 +/− 0.06 | 0.8 +/− 0.07 | 1.0 +/− 0.03 |
| CACNB3 | 601958 | 34726_at | 0.7 +/− 0.11 | 0.5 +/− 0.06 | 0.9 +/− 0.19 | 0.7 +/− 0.17 | 0.9 +/− 0.10 |
| CASP1 | 147678 | 209970_x_at | 4.8 +/− 0.91 | 10.8 +/− 0.64 | 1.0 +/− 0.32 | 5.6 +/− 1.09 | 0.8 +/− 0.20 |
| CASP1 | 147678 | 211366_x_at | 4.3 +/− 0.61 | 7.9 +/− 0.60 | 1.2 +/− 0.17 | 4.9 +/− 0.87 | 0.8 +/− 0.47 |
| CASP1 | 147678 | 211367_s_at | 21.3 +/− 3.58 | 43.6 +/− 7.81 | 1.2 +/− 1.04 | 19.0 +/− 3.34 | 2.5 +/− 1.31 |
| CASP1 | 147678 | 211368_s_at | 81.2 +/− 18.16 | 223.4 +/− 34.81 | 1.9 +/− 0.72 | 92.8 +/− 20.86 | 1.9 +/− 0.49 |
| CASP4 | 602664 | 209310_s_at | 1.9 +/− 0.15 | 2.6 +/− 0.26 | 1.1 +/− 0.13 | 1.8 +/− 0.32 | 1.1 +/− 0.19 |
| CASP7 | 601761 | 207181_s_at | 1.4 +/− 0.13 | 1.9 +/− 0.23 | 1.1 +/− 0.20 | 1.5 +/− 0.24 | 1.2 +/− 0.12 |
| CBR3 | 603608 | 205379_at | 1.6 +/− 0.12 | 2.2 +/− 0.22 | 1.0 +/− 0.04 | 1.5 +/− 0.19 | 0.9 +/− 0.07 |
| CCRL1 | 606065 | 220351_at | 1.1 +/− 0.39 | 2.4 +/− 0.24 | 1.2 +/− 0.24 | 1.5 +/− 0.18 | 1.3 +/− 0.29 |
| CCT8 | — | 200873_s_at | 1.1 +/− 0.04 | 1.2 +/− 0.06 | 1.0 +/− 0.02 | 1.0 +/− 0.04 | 1.0 +/− 0.02 |
| CD109 | — | 226545_at | 0.8 +/− 0.07 | 0.7 +/− 0.03 | 0.9 +/− 0.07 | 0.8 +/− 0.06 | 0.9 +/− 0.04 |
| CD151 | 602243 | 204306_s_at | 0.9 +/− 0.06 | 0.8 +/− 0.04 | 1.0 +/− 0.05 | 0.9 +/− 0.04 | 0.9 +/− 0.01 |
| CD38 | 107270 | 205692_a_at | 1.7 +/− 0.37 | 2.1 +/− 0.43 | 1.1 +/− 0.45 | 1.6 +/− 0.30 | 1.0 +/− 0.15 |
| CD47 | 601028 | 213857_s_at | 1.4 +/− 0.08 | 1.8 +/− 0.21 | 1.0 +/− 0.09 | 1.4 +/− 0.15 | 0.9 +/− 0.04 |
| CD47 | 601028 | 226016_at | 1.2 +/− 0.10 | 1.5 +/− 0.13 | 1.0 +/− 0.12 | 1.2 +/− 0.07 | 1.0 +/− 0.04 |
| CD47 | 601028 | 211075_s_at | 1.5 +/− 0.07 | 1.8 +/− 0.12 | 1.1 +/− 0.06 | 1.4 +/− 0.09 | 1.1 +/− 0.03 |
| CD9 | 143030 | 201005_at | 0.7 +/− 0.11 | 0.6 +/− 0.10 | 1.0 +/− 0.16 | 0.7 +/− 0.14 | 1.0 +/− 0.14 |
| CDC42EP3 | 606133 | 209286_at | 0.7 +/− 0.04 | 0.7 +/− 0.06 | 1.0 +/− 0.05 | 0.8 +/− 0.06 | 1.0 +/− 0.05 |
| CDC42EP4 | 605468 | 214721_x_at | 1.2 +/− 0.14 | 1.4 +/− 0.03 | 1.1 +/− 0.09 | 1.3 +/− 0.12 | 1.0 +/− 0.05 |
| CDC42EP4 | 605468 | 218062_x_at | 1.1 +/− 0.05 | 1.3 +/− 0.12 | 1.0 +/− 0.07 | 1.1 +/− 0.10 | 0.9 +/− 0.05 |
| CEACAM1 | 109770 | 206576_s_at | 1.6 +/− 0.39 | 2.2 +/− 0.13 | 1.4 +/− 0.20 | 1.6 +/− 0.40 | 1.4 +/− 0.27 |
| CEACAM1 | 109770 | 209498_at | 3.6 +/− 0.85 | 7.8 +/− 0.91 | 1.2 +/− 0.30 | 4.3 +/− 0.93 | 1.1 +/− 0.96 |
| CEACAM1 | 109770 | 211889_x_at | 2.0 +/− 0.51 | 3.2 +/− 0.21 | 1.1 +/− 0.11 | 1.9 +/− 0.35 | 1.9 +/− 0.60 |
| CEBPB | 189965 | 212501_at | 1.1 +/− 0.03 | 1.3 +/− 0.06 | 1.1 +/− 0.03 | 1.1 +/− 0.03 | 1.1 +/− 0.04 |
| CENTA1 | 608114 | 90265_at | 1.0 0.22 | 1.7 0.36 | 1.1 0.22 | 1.5 0.16 | 0.9 0.15 |
| CERK | — | 218421_at | 0.7 +/− 0.08 | 0.6 +/− 0.06 | 0.9 +/− 0.12 | 0.9 +/− 0.05 | 0.9 +/− 0.03 |
| CGI-48 | — | 203721_s_at | 1.0 +/− 0.03 | 1.1 +/− 0.02 | 1.0 +/− 0.04 | 1.1 +/− 0.03 | 1.0 +/− 0.06 |
| CKB | 123280 | 200884_at | 1.0 +/− 0.15 | 0.8 +/− 0.08 | 1.1 +/− 0.11 | 0.8 +/− 0.05 | 1.1 +/− 0.04 |
| CKLF | — | 223451_s_at | 0.9 +/− 0.02 | 0.9 +/− 0.05 | 1.0 +/− 0.03 | 0.9 +/− 0.06 | 1.0 +/− 0.05 |
| CLECSF2 | 603242 | 209732_at | 1.7 +/− 0.30 | 2.3 +/− 0.09 | 1.2 +/− 0.14 | 1.8 +/− 0.43 | 1.2 +/− 0.16 |
| CLMN | — | 221042_s_at | 0.8 +/− 0.09 | 0.7 +/− 0.05 | 0.8 +/− 0.09 | 0.7 +/− 0.02 | 0.9 +/− 0.08 |
| CLSTN1 | — | 201561_s_at | 0.8 +/− 0.04 | 0.7 +/− 0.04 | 1.0 +/− 0.08 | 0.8 +/− 0.08 | 1.0 +/− 0.04 |
| CLTB | 118970 | 206284_x_at | 0.9 +/− 0.05 | 0.8 +/− 0.11 | 0.9 +/− 0.08 | 0.8 +/− 0.05 | 1.0 +/− 0.08 |
| CLTB | 118970 | 211043_s_at | 0.9 +/− 0.05 | 0.7 +/− 0.03 | 1.0 +/− 0.08 | 0.9 +/− 0.10 | 0.9 +/− 0.04 |
| CML66 | 606109 | 225438_at | 1.0 +/− 0.10 | 1.2 +/− 0.11 | 0.9 +/− 0.14 | 1.0 +/− 0.15 | 0.8 +/− 0.08 |
| CML66 | 606109 | 225439_at | 1.1 +/− 0.04 | 1.3 +/− 0.05 | 1.1 +/− 0.06 | 1.1 +/− 0.03 | 1.1 +/− 0.06 |
| CNTN1 | 600016 | 227202_at | 0.7 +/− 0.07 | 0.6 +/− 0.06 | 0.9 +/− 0.10 | 0.8 +/− 0.10 | 0.9 +/− 0.11 |
| CNTN1 | 600016 | 227209_at | 0.8 +/− 0.08 | 0.6 +/− 0.06 | 1.0 +/− 0.10 | 0.8 +/− 0.10 | 1.0 +/− 0.07 |
| COP | — | 1552703_s_at | 7.3 +/− 2.30 | 16.7 +/− 3.94 | 1.6 +/− 0.72 | 7.9 +/− 1.87 | 1.3 +/− 0.35 |
| CREM | 123812 | 209967_s_at | 1.2 +/− 0.07 | 1.4 +/− 0.15 | 0.9 +/− 0.14 | 1.2 +/− 0.07 | 0.9 +/− 0.07 |
| CSF1 | 120420 | 209716_at | 1.3 +/− 0.21 | 1.6 +/− 0.12 | 1.0 +/− 0.20 | 1.2 +/− 0.17 | 0.9 +/− 0.20 |
| CST | 602300 | 205670_at | 0.6 +/− 0.03 | 0.4 +/− 0.13 | 0.9 +/− 0.11 | 0.7 +/− 0.12 | 0.8 +/− 0.09 |
| CTH | 607657 | 217127_at | 1.6 +/− 0.30 | 2.1 +/− 0.27 | 1.1 +/− 0.08 | 1.6 +/− 0.23 | 1.1 +/− 0.13 |
| CTSC | 602365 | 201487_at | 1.1 +/− 0.10 | 1.3 +/− 0.05 | 1.0 +/− 0.06 | 1.2 +/− 0.07 | 1.0 +/− 0.05 |
| CTSL | 116880 | 202087_s_at | 1.3 +/− 0.11 | 1.5 +/− 0.15 | 1.2 +/− 0.16 | 1.2 +/− 0.05 | 1.1 +/− 0.06 |
| CTSL2 | 603308 | 210074_at | 0.8 +/− 0.05 | 0.8 +/− 0.03 | 1.0 +/− 0.08 | 0.9 +/− 0.08 | 1.0 +/− 0.09 |
| CTSS | 116845 | 202901_x_at | 3.9 +/− 0.78 | 6.4 +/− 1.08 | 1.1 +/− 0.18 | 4.1 +/− 1.03 | 1.1 +/− 0.10 |
| CTSS | 116845 | 202902_s_at | 2.3 +/− 0.26 | 4.5 +/− 0.82 | 1.2 +/− 0.40 | 3.1 +/− 1.34 | 1.2 +/− 0.07 |
| CTSS | 116845 | 232617_at | 2.6 +/− 0.18 | 4.2 +/− 0.58 | 1.2 +/− 0.16 | 2.6 +/− 0.12 | 1.3 +/− 0.35 |
| CXCL16 | 605398 | 223454_at | 1.3 +/− 0.17 | 1.4 +/− 0.09 | 1.1 +/− 0.21 | 1.3 +/− 0.09 | 0.9 +/− 0.09 |
| CXCL2 | 139110 | 209774_x_at | 1.3 +/− 0.13 | 1.7 +/− 0.21 | 1.2 +/− 0.13 | 1.2 +/− 0.15 | 1.1 +/− 0.06 |
| CXX1 | 300213 | 201828_x_at | 0.9 +/− 0.07 | 0.8 +/− 0.07 | 1.0 +/− 0.08 | 0.8 +/− 0.05 | 0.9 +/− 0.03 |
| CYT19 | — | 223652_at | 0.9 +/− 0.08 | 0.6 +/− 0.11 | 1.0 +/− 0.08 | 0.9 +/− 0.14 | 1.1 +/− 0.07 |
| DAAM1 | 606626 | 216060_s_at | 0.6 +/− 0.12 | 0.6 +/− 0.08 | 0.8 +/− 0.07 | 0.7 +/− 0.16 | 0.7 +/− 0.10 |
| DAF | 125240 | 201925_s_at | 1.2 +/− 0.11 | 1.4 +/− 0.07 | 1.0 +/− 0.07 | 1.3 +/− 0.16 | 1.0 +/− 0.05 |
| DAP4 | — | 202572_s_at | 0.9 +/− 0.08 | 0.8 +/− 0.04 | 0.8 +/− 0.07 | 0.9 +/− 0.04 | 0.8 +/− 0.03 |
| DCDC2 | 605755 | 222925_at | 0.9 +/− 0.03 | 0.8 +/− 0.02 | 0.9 +/− 0.12 | 0.9 +/− 0.06 | 1.0 +/− 0.04 |
| DEFB1 | 602056 | 210397_at | 0.8 +/− 0.11 | 0.6 +/− 0.10 | 1.0 +/− 0.14 | 0.7 +/− 0.10 | 1.0 +/− 0.15 |
| DIP13B | 606231 | 218218_at | 0.9 +/− 0.06 | 0.7 +/− 0.04 | 1.1 +/− 0.04 | 0.9 +/− 0.04 | 1.0 +/− 0.07 |
| DIRC2 | 602773 | 226026_at | 0.8 +/− 0.04 | 0.7 +/− 0.04 | 0.9 +/− 0.05 | 0.9 +/− 0.04 | 1.0 +/− 0.03 |
| DJ473B4 | — | 218853_s_at | 0.8 +/− 0.08 | 0.7 +/− 0.05 | 0.9 +/− 0.07 | 0.9 +/− 0.07 | 1.0 +/− 0.11 |
| DKFZP434B | — | 233092_s_at | 0.7 +/− 0.06 | 0.7 +/− 0.06 | 0.8 +/− 0.11 | 0.8 +/− 0.04 | 0.8 +/− 0.06 |
| [symbol] | | | | | | | |
| DKFZp434D | — | 236273_at | 0.7 +/− 0.06 | 0.6 +/− 0.06 | 0.9 +/− 0.11 | 0.9 +/− 0.10 | 0.9 +/− 0.11 |
| [symbol] | | | | | | | |
| DNAJA1 | 602837 | 200880_at | 1.1 +/− 0.05 | 1.2 +/− 0.04 | 1.1 +/− 0.07 | 1.1 +/− 0.05 | 1.0 +/− 0.02 |
| DNM1 | 602377 | 215116_s_at | 0.9 +/− 0.05 | 0.7 +/− 0.06 | 1.0 +/− 0.07 | 0.9 +/− 0.13 | 0.9 +/− 0.03 |
| DNPEP | — | 201937_s_at | 1.7 +/− 0.03 | 2.3 +/− 0.20 | 1.0 +/− 0.09 | 1.8 +/− 0.18 | 1.0 +/− 0.05 |
| DNPEP | — | 38703_at | 1.5 +/− 0.14 | 1.9 +/− 0.38 | 1.0 +/− 0.13 | 1.5 +/− 0.15 | 1.0 +/− 0.05 |
| DOCK4 | 607679 | 205003_at | 1.1 +/− 0.08 | 1.5 +/− 0.13 | 1.0 +/− 0.11 | 1.3 +/− 0.13 | 1.0 +/− 0.18 |
| DPYD | 274270 | 204646_at | 1.5 +/− 0.20 | 2.2 +/− 0.22 | 1.0 +/− 0.14 | 1.7 +/− 0.28 | 0.9 +/− 0.19 |
| DUSP16 | 607175 | 224832_at | 1.4 +/− 0.17 | 1.5 +/− 0.07 | 1.1 +/− 0.12 | 1.3 +/− 0.08 | 1.1 +/− 0.09 |
| ECGF1 | 131222 | 204858_s_at | 1.9 +/− 0.19 | 2.1 +/− 0.34 | 1.4 +/− 0.14 | 1.6 +/− 0.24 | 1.2 +/− 0.14 |
| EDG3 | 601965 | 228176_at | 1.0 +/− 0.02 | 0.8 +/− 0.07 | 1.0 +/− 0.09 | 0.9 +/− 0.04 | 1.0 +/− 0.05 |
| EMP2 | 602334 | 204975_at | 0.9 +/− 0.08 | 0.8 +/− 0.05 | 1.0 +/− 0.06 | 0.9 +/− 0.05 | 1.0 +/− 0.04 |
| EMR1 | 600493 | 207111_at | 2.4 +/− 0.38 | 2.6 +/− 0.38 | 1.6 +/− 0.26 | 2.1 +/− 0.22 | 1.2 +/− 0.13 |
| EPB41L1 | 602879 | 212336_at | 0.9 +/− 0.08 | 0.7 +/− 0.06 | 0.9 +/− 0.07 | 0.8 +/− 0.10 | 0.9 +/− 0.10 |
| EPPB9 | — | 210534_s_at | 0.8 +/− 0.07 | 0.7 +/− 0.02 | 1.0 +/− 0.14 | 0.8 +/− 0.04 | 1.0 +/− 0.05 |
| F3 | 134390 | 204363_at | 0.7 +/− 0.09 | 0.6 +/− 0.03 | 1.0 +/− 0.11 | 0.7 +/− 0.12 | 1.0 +/− 0.16 |
| FACL3 | 602371 | 201662_s_at | 1.1 +/− 0.12 | 1.3 +/− 0.05 | 0.9 +/− 0.10 | 1.1 +/− 0.14 | 0.9 +/− 0.05 |
| FACL5 | 605677 | 218322_s_at | 1.5 +/− 0.20 | 2.0 +/− 0.17 | 1.1 +/− 0.24 | 1.4 +/− 0.11 | 1.2 +/− 0.09 |
| FACL5 | 605677 | 222592_s_at | 7.0 +/− 2.81 | 13.6 +/− 3.87 | 1.0 +/− 0.65 | 6.1 +/− 1.26 | 0.9 +/− 0.69 |
| FAXDC1 | — | 219429_at | 0.9 +/− 0.10 | 0.7 +/− 0.01 | 1.0 +/− 0.09 | 0.8 +/− 0.09 | 1.0 +/− 0.07 |
| FBLP-1 | 607747 | 225258_at | 0.7 +/− 0.10 | 0.6 +/− 0.07 | 0.8 +/− 0.20 | 0.8 +/− 0.09 | 0.9 +/− 0.14 |
| FBXO6 | 605647 | 231769_at | 1.9 +/− 0.18 | 2.5 +/− 0.24 | 1.2 +/− 0.16 | 1.8 +/− 0.22 | 1.3 +/− 0.08 |
| FBXO9 | — | 210638_s_at | 0.9 +/− 0.04 | 0.8 +/− 0.02 | 0.9 +/− 0.07 | 0.9 +/− 0.03 | 0.9 +/− 0.01 |
| FDFT1 | 184420 | 210950_s_at | 1.0 +/− 0.08 | 0.9 +/− 0.07 | 1.1 +/− 0.11 | 1.0 +/− 0.05 | 1.1 +/− 0.04 |
| FKBP11 | — | 219117_s_at | 1.6 +/− 0.15 | 1.8 +/− 0.08 | 1.3 +/− 0.18 | 1.5 +/− 0.13 | 1.2 +/− 0.17 |
| FKBP11 | — | 219118_at | 1.3 +/− 0.13 | 1.5 +/− 0.14 | 1.0 +/− 0.15 | 1.3 +/− 0.10 | 1.0 +/− 0.15 |
| FKBP11 | — | 228308_at | 1.3 +/− 0.08 | 1.3 +/− 0.14 | 0.9 +/− 0.13 | 1.1 +/− 0.08 | 1.0 +/− 0.08 |
| FLJ10116 | — | 219648_at | 1.3 +/− 0.15 | 1.8 +/− 0.17 | 1.0 +/− 0.03 | 1.4 +/− 0.12 | 1.1 +/− 0.11 |
| FLJ10276 | — | 222200_s_at | 0.8 +/− 0.05 | 0.7 +/− 0.07 | 0.9 +/− 0.07 | 0.9 +/− 0.10 | 0.9 +/− 0.05 |
| FLJ10381 | — | 218396_at | 1.1 +/− 0.07 | 1.3 +/− 0.16 | 0.9 +/− 0.08 | 1.1 +/− 0.07 | 1.0 +/− 0.08 |
| FLJ10525 | — | 218465_at | 1.0 +/− 0.03 | 1.1 +/− 0.07 | 0.9 +/− 0.04 | 1.1 +/− 0.08 | 0.9 +/− 0.04 |
| FLJ10579 | — | 218126_at | 1.0 +/− 0.02 | 1.2 +/− 0.05 | 1.0 +/− 0.03 | 1.0 +/− 0.04 | 0.9 +/− 0.06 |
| FLJ10980 | — | 225327_at | 0.7 +/− 0.11 | 0.5 +/− 0.05 | 0.8 +/− 0.12 | 0.8 +/− 0.11 | 0.9 +/− 0.18 |
| FLJ11259 | — | 218627_at | 1.3 +/− 0.12 | 2.0 +/− 0.15 | 0.9 +/− 0.17 | 1.3 +/− 0.16 | 0.9 +/− 0.13 |
| FLJ11294 | — | 222763_s_at | 0.9 +/− 0.06 | 0.8 +/− 0.07 | 1.0 +/− 0.08 | 0.9 +/− 0.03 | 1.0 +/− 0.05 |
| FLJ11342 | — | 218633_x_at | 0.9 +/− 0.08 | 0.8 +/− 0.08 | 1.0 +/− 0.05 | 0.8 +/− 0.05 | 1.0 +/− 0.05 |
| FLJ12150 | — | 1556242_a_at | 1.2 +/− 0.05 | 1.4 +/− 0.12 | 0.9 +/− 0.04 | 1.1 +/− 0.06 | 0.9 +/− 0.16 |
| FLJ12150 | — | 218154_at | 2.0 +/− 0.44 | 2.2 +/− 0.14 | 1.3 +/− 0.30 | 1.9 +/− 0.32 | 1.0 +/− 0.18 |
| FLJ12442 | — | 218051_s_at | 0.8 +/− 0.09 | 0.6 +/− 0.07 | 0.7 +/− 0.07 | 0.8 +/− 0.11 | 0.8 +/− 0.09 |
| FLJ12505 | — | 235343_at | 2.3 +/− 0.77 | 3.9 +/− 0.76 | 1.2 +/− 0.36 | 2.6 +/− 0.90 | 1.1 +/− 0.37 |
| FLJ12584 | — | 219637_at | 0.8 +/− 0.13 | 0.6 +/− 0.07 | 1.2 +/− 0.21 | 0.7 +/− 0.22 | 0.9 +/− 0.06 |
| FLJ12953 | — | 225898_at | 0.9 +/− 0.06 | 0.8 +/− 0.06 | 1.0 +/− 0.04 | 0.9 +/− 0.05 | 1.0 +/− 0.05 |
| FLJ14054 | — | 219054_at | 1.1 +/− 0.05 | 1.2 +/− 0.06 | 1.0 +/− 0.06 | 1.1 +/− 0.04 | 1.0 +/− 0.04 |
| FLJ14464 | — | 229350_x_at | 20.5 +/− 3.26 | 43.3 +/− 10.13 | 4.2 +/− 2.09 | 16.4 +/− 3.97 | 1.8 +/− 0.62 |
| FLJ20013 | — | 231406_at | 0.8 +/− 0.05 | 0.5 +/− 0.09 | 1.1 +/− 0.11 | 0.7 +/− 0.16 | 1.0 +/− 0.14 |
| FLJ20035 | — | 218986_s_at | 6.6 +/− 0.70 | 5.8 +/− 0.32 | 4.6 +/− 0.38 | 3.6 +/− 0.36 | 3.0 +/− 0.64 |
| FLJ20073 | — | 219691_at | 2.5 +/− 0.40 | 2.7 +/− 0.68 | 3.1 +/− 0.08 | 2.0 +/− 0.33 | 2.3 +/− 0.51 |
| FLJ20073 | — | 228531_at | 2.3 +/− 0.38 | 2.0 +/− 0.39 | 2.7 +/− 0.34 | 1.5 +/− 0.30 | 1.5 +/− 0.54 |
| FLJ20156 | — | 219338_s_at | 0.8 +/− 0.11 | 0.7 +/− 0.05 | 1.0 +/− 0.06 | 0.9 +/− 0.06 | 0.9 +/− 0.10 |
| FLJ20160 | — | 225325_at | 0.8 +/− 0.04 | 0.7 +/− 0.08 | 1.0 +/− 0.10 | 0.9 +/− 0.05 | 1.0 +/− 0.08 |
| FLJ20366 | — | 218692_at | 0.8 +/− 0.07 | 0.8 +/− 0.08 | 1.0 +/− 0.06 | 0.9 +/− 0.10 | 1.0 +/− 0.06 |
| FLJ20637 | — | 219352_at | 1.9 +/− 0.39 | 1.4 +/− 0.11 | 3.0 +/− 0.46 | 1.2 +/− 0.06 | 1.7 +/− 0.36 |
| FLJ20651 | — | 222708_s_at | 1.1 +/− 0.09 | 1.2 +/− 0.11 | 1.0 +/− 0.03 | 1.2 +/− 0.10 | 0.9 +/− 0.02 |
| FLJ20651 | — | 226662_at | 1.0 +/− 0.09 | 1.1 +/− 0.05 | 0.9 +/− 0.06 | 1.2 +/− 0.10 | 0.9 +/− 0.06 |
| FLJ20651 | — | 228091_at | 1.1 +/− 0.04 | 1.3 +/− 0.12 | 1.0 +/− 0.08 | 1.3 +/− 0.13 | 1.0 +/− 0.04 |
| FLJ21347 | — | 218164_at | 0.8 +/− 0.07 | 0.7 +/− 0.04 | 0.9 +/− 0.06 | 0.8 +/− 0.12 | 0.9 +/− 0.07 |
| FLJ22573 | — | 219690_at | 1.8 +/− 0.19 | 2.6 +/− 0.20 | 1.0 +/− 0.14 | 1.7 +/− 0.25 | 0.9 +/− 0.09 |
| FLJ22693 | — | 218543_s_at | 2.3 +/− 0.35 | 2.7 +/− 0.28 | 2.0 +/− 0.37 | 2.1 +/− 0.32 | 1.4 +/− 0.17 |
| FLJ22794 | — | 218248_at | 1.2 +/− 0.08 | 1.4 +/− 0.03 | 1.1 +/− 0.15 | 1.2 +/− 0.06 | 1.0 +/− 0.19 |
| FLJ23309 | — | 222603_at | 0.8 +/− 0.08 | 0.7 +/− 0.05 | 0.9 +/− 0.07 | 0.8 +/− 0.06 | 0.9 +/− 0.03 |
| FLJ23654 | — | 243582_at | 0.7 +/− 0.04 | 0.6 +/− 0.13 | 0.9 +/− 0.07 | 0.7 +/− 0.07 | 0.9 +/− 0.04 |
| FLJ23878 | — | 1553315_at | 1.0 +/− 0.11 | 1.3 +/− 0.40 | 0.5 +/− 0.21 | 1.4 +/− 0.10 | 1.0 +/− 0.21 |
| FLJ31033 | — | 228152_s_at | 2.3 +/− 0.17 | 2.9 +/− 0.26 | 1.7 +/− 0.22 | 2.2 +/− 0.21 | 1.2 +/− 0.22 |
| FLJ31842 | — | 234980_at | 0.7 +/− 0.09 | 0.7 +/− 0.08 | 0.9 +/− 0.11 | 0.7 +/− 0.05 | 0.9 +/− 0.02 |
| FLJ32122 | — | 226875_at | 0.8 +/− 0.12 | 0.6 +/− 0.04 | 1.0 +/− 0.12 | 0.8 +/− 0.11 | 1.0 +/− 0.11 |
| FLJ33718 | — | 240633_at | 0.8 +/− 0.12 | 0.8 +/− 0.19 | 1.2 +/− 0.20 | 0.9 +/− 0.05 | 1.1 +/− 0.14 |
| FLJ34233 | — | 226785_at | 1.2 +/− 0.06 | 1.2 +/− 0.10 | 1.0 +/− 0.11 | 1.3 +/− 0.06 | 1.0 +/− 0.12 |
| FLJ34389 | — | 238025_at | 1.1 +/− 0.06 | 1.3 +/− 0.10 | 1.1 +/− 0.03 | 1.1 +/− 0.07 | 1.1 +/− 0.06 |
| FLJ36874 | — | 225466_at | 1.1 +/− 0.08 | 1.2 +/− 0.04 | 0.9 +/− 0.04 | 1.0 +/− 0.08 | 0.9 +/− 0.10 |
| FLJ38348 | — | 213294_at | 1.5 +/− 0.10 | 1.4 +/− 0.09 | 1.8 +/− 0.03 | 1.3 +/− 0.06 | 1.4 +/− 0.03 |
| FLJ38348 | — | 238743_at | 1.5 +/− 0.31 | 1.7 +/− 0.30 | 1.8 +/− 0.20 | 1.2 +/− 0.25 | 1.7 +/− 0.25 |
| FLJ39553 | — | 1552389_at | 0.7 +/− 0.32 | 0.4 +/− 0.05 | 0.8 +/− 0.14 | 0.4 +/− 0.19 | 0.7 +/− 0.19 |
| FLJ39553 | — | 1552390_a_at | 0.6 +/− 0.13 | 0.7 +/− 0.03 | 0.9 +/− 0.10 | 0.6 +/− 0.06 | 0.9 +/− 0.01 |
| FLJ39885 | — | 226603_at | 2.5 +/− 0.26 | 4.2 +/− 0.28 | 1.5 +/− 0.21 | 2.1 +/− 0.28 | 1.2 +/− 0.20 |
| FLJ39885 | — | 230036_at | 4.3 +/− 2.13 | 10.8 +/− 1.24 | 2.7 +/− 1.22 | 5.1 +/− 1.47 | 3.1 +/− 2.13 |
| FMR1 | 309550 | 203689_s_at | 1.1 +/− 0.10 | 1.4 +/− 0.16 | 1.0 +/− 0.14 | 1.2 +/− 0.05 | 0.9 +/− 0.08 |
| FRABIN | — | 227948_at | 0.7 +/− 0.11 | 0.6 +/− 0.14 | 0.9 +/− 0.14 | 0.7 +/− 0.02 | 1.0 +/− 0.12 |
| FRABIN | — | 230559_x_at | 0.6 +/− 0.10 | 0.5 +/− 0.09 | 1.1 +/− 0.11 | 0.8 +/− 0.12 | 1.1 +/− 0.22 |
| FRABIN | — | 242445_at | 1.1 +/− 0.17 | 0.7 +/− 0.17 | 0.9 +/− 0.28 | 0.9 +/− 0.30 | 1.6 +/− 0.13 |
| FTH1 | 134770 | 214211_at | 0.9 +/− 0.03 | 0.8 +/− 0.04 | 1.0 +/− 0.09 | 0.9 +/− 0.03 | 1.0 +/− 0.06 |
| FXYD5 | 606669 | 218084_x_at | 0.9 +/− 0.04 | 0.8 +/− 0.05 | 1.0 +/− 0.02 | 0.8 +/− 0.03 | 1.0 +/− 0.03 |
| FZD6 | 603409 | 203987_at | 0.8 +/− 0.06 | 0.7 +/− 0.08 | 1.0 +/− 0.08 | 0.9 +/− 0.08 | 1.0 +/− 0.06 |
| G1P2 | 147571 | 205483_s_at | 13.9 +/− 1.84 | 12.1 +/− 0.58 | 26.7 +/− 3.44 | 5.8 +/− 0.72 | 8.4 +/− 1.19 |
| G1P3 | 147572 | 204415_at | 9.7 +/− 1.12 | 4.7 +/− 0.52 | 15.0 +/− 1.96 | 3.1 +/− 1.02 | 11.1 +/− 0.69 |
| GALNT10 | 608043 | 207357_s_at | 0.8 +/− 0.11 | 0.7 +/− 0.09 | 1.0 +/− 0.06 | 0.8 +/− 0.08 | 1.0 +/− 0.06 |
| GBP1 | 600411 | 202269_x_at | 10.0 +/− 1.67 | 17.5 +/− 2.52 | 1.1 +/− 0.25 | 7.7 +/− 0.74 | 0.9 +/− 0.24 |
| GBP1 | 600411 | 202270_at | 17.9 +/− 2.34 | 41.9 +/− 3.59 | 1.8 +/− 0.35 | 19.1 +/− 3.36 | 1.5 +/− 0.54 |
| GBP1 | 600411 | 231577_s_at | 29.1 +/− 5.31 | 56.9 +/− 9.36 | 1.6 +/− 0.25 | 25.4 +/− 3.32 | 1.6 +/− 0.61 |
| GBP2 | 600412 | 202748_at | 3.1 +/− 0.21 | 4.5 +/− 0.16 | 0.9 +/− 0.09 | 2.7 +/− 0.18 | 1.1 +/− 0.18 |
| GBP2 | 600412 | 242907_at | 2.1 +/− 0.25 | 4.8 +/− 0.59 | 0.8 +/− 0.46 | 2.1 +/− 0.07 | 1.2 +/− 0.37 |
| GBP3 | 600413 | 223434_at | 5.8 +/− 0.70 | 11.6 +/− 0.75 | 1.4 +/− 0.24 | 6.3 +/− 0.77 | 1.3 +/− 0.15 |
| GDA | 139260 | 224209_s_at | 0.9 +/− 0.08 | 0.7 +/− 0.05 | 1.1 +/− 0.12 | 0.9 +/− 0.13 | 1.1 +/− 0.08 |
| GLA | 301500 | 214430_at | 1.1 +/− 0.04 | 1.3 +/− 0.10 | 1.0 +/− 0.03 | 1.1 +/− 0.05 | 1.0 +/− 0.05 |
| GLS | 138280 | 203157_s_at | 0.6 +/− 0.11 | 0.5 +/− 0.06 | 0.8 +/− 0.11 | 0.6 +/− 0.06 | 0.8 +/− 0.07 |
| GLS | 138280 | 203159_at | 0.6 +/− 0.10 | 0.6 +/− 0.09 | 0.8 +/− 0.11 | 0.6 +/− 0.03 | 0.9 +/− 0.10 |
| GNA13 | 604406 | 224761_at | 1.0 +/− 0.06 | 1.1 +/− 0.02 | 1.0 +/− 0.05 | 1.0 +/− 0.03 | 1.0 +/− 0.04 |
| GPR56 | 604110 | 212070_at | 0.8 +/− 0.06 | 0.5 +/− 0.07 | 1.1 +/− 0.13 | 0.7 +/− 0.05 | 1.1 +/− 0.09 |
| GPRC5C | 605949 | 219327_s_at | 0.7 +/− 0.02 | 0.6 +/− 0.13 | 1.0 +/− 0.08 | 0.8 +/− 0.15 | 0.9 +/− 0.06 |
| GRPEL1 | 606173 | 212434_at | 1.2 +/− 0.02 | 1.2 +/− 0.06 | 1.0 +/− 0.08 | 1.1 +/− 0.02 | 1.0 +/− 0.04 |
| GSN | 137350 | 200696_s_at | 0.8 +/− 0.05 | 0.7 +/− 0.13 | 0.9 +/− 0.11 | 0.7 +/− 0.14 | 1.0 +/− 0.12 |
| GSTO1 | 605482 | 1557915_s_at | 1.3 +/− 0.09 | 1.4 +/− 0.08 | 1.0 +/− 0.15 | 1.1 +/− 0.10 | 1.1 +/− 0.15 |
| GSTO1 | 605482 | 201470_at | 1.2 +/− 0.05 | 1.4 +/− 0.08 | 1.0 +/− 0.12 | 1.1 +/− 0.09 | 1.0 +/− 0.16 |
| GTPBP1 | 602245 | 219357_at | 1.2 +/− 0.10 | 1.4 +/− 0.08 | 1.1 +/− 0.17 | 1.2 +/− 0.27 | 0.8 +/− 0.09 |
| GUK1 | 139270 | 200075_s_at | 1.5 +/− 0.19 | 1.7 +/− 0.12 | 1.0 +/− 0.09 | 1.3 +/− 0.04 | 1.1 +/− 0.07 |
| HAPLN3 | — | 227262_at | 1.6 +/− 0.19 | 2.6 +/− 0.41 | 1.0 +/− 0.10 | 1.6 +/− 0.18 | 1.0 +/− 0.12 |
| HBP17 | 607737 | 205014_at | 0.7 +/− 0.20 | 0.5 +/− 0.17 | 1.1 +/− 0.19 | 0.8 +/− 0.13 | 1.1 +/− 0.15 |
| HF1 | 134370 | 213800_at | 1.4 +/− 0.18 | 1.8 +/− 0.13 | 1.1 +/− 0.29 | 1.3 +/− 0.21 | 1.1 +/− 0.17 |
| HFL1 | 134371 | 215388_s_at | 1.8 +/− 0.31 | 2.5 +/− 0.20 | 1.1 +/− 0.18 | 1.7 +/− 0.30 | 1.0 +/− 0.19 |
| HHGP | — | 222803_at | 0.9 +/− 0.07 | 0.8 +/− 0.03 | 1.0 +/− 0.03 | 1.0 +/− 0.02 | 0.9 +/− 0.05 |
| HIC2 | 607712 | 212966_at | 1.2 +/− 0.10 | 1.1 +/− 0.11 | 0.9 +/− 0.08 | 1.0 +/− 0.05 | 0.9 +/− 0.06 |
| HIP1 | 601767 | 205425_at | 0.8 +/− 0.06 | 0.7 +/− 0.15 | 1.0 +/− 0.04 | 0.8 +/− 0.15 | 1.0 +/− 0.05 |
| HIP1 | 601767 | 226364_at | 0.7 +/− 0.16 | 0.6 +/− 0.07 | 1.0 +/− 0.17 | 0.8 +/− 0.09 | 1.0 +/− 0.12 |
| HIP14L | — | 219296_at | 0.8 +/− 0.02 | 0.8 +/− 0.03 | 1.0 +/− 0.11 | 0.9 +/− 0.07 | 1.0 +/− 0.06 |
| HIS1 | 607328 | 202814_s_at | 1.0 +/− 0.04 | 1.1 +/− 0.06 | 1.0 +/− 0.06 | 1.1 +/− 0.02 | 0.9 +/− 0.03 |
| HLA-A | 142800 | 213932_x_at | 3.9 +/− 0.41 | 4.8 +/− 0.72 | 1.4 +/− 0.17 | 3.8 +/− 0.35 | 1.1 +/− 0.10 |
| HLA-A | 142800 | 215313_x_at | 3.0 +/− 0.10 | 3.9 +/− 0.07 | 1.3 +/− 0.03 | 3.0 +/− 0.28 | 1.1 +/− 0.06 |
| HLA-B | 142830 | 208729_x_at | 4.2 +/− 0.67 | 6.2 +/− 0.68 | 1.1 +/− 0.20 | 3.6 +/− 0.32 | 1.0 +/− 0.21 |
| HLA-B | 142830 | 209140_x_at | 6.0 +/− 0.76 | 8.0 +/− 0.51 | 1.7 +/− 0.23 | 5.3 +/− 0.21 | 1.2 +/− 0.16 |
| HLA-B | 142830 | 211911_x_at | 4.9 +/− 0.84 | 6.9 +/− 0.50 | 1.6 +/− 0.21 | 4.4 +/− 0.37 | 1.3 +/− 0.16 |
| HLA-C | 142840 | 208812_x_at | 4.2 +/− 0.34 | 5.5 +/− 0.08 | 1.7 +/− 0.20 | 3.9 +/− 0.16 | 1.2 +/− 0.14 |
| HLA-C | 142840 | 211799_x_at | 4.3 +/− 0.49 | 6.7 +/− 0.91 | 1.1 +/− 0.15 | 4.2 +/− 0.48 | 1.0 +/− 0.10 |
| HLA-C | 142840 | 214459_x_at | 4.0 +/− 0.21 | 4.9 +/− 0.26 | 1.6 +/− 0.16 | 3.3 +/− 0.18 | 1.2 +/− 0.11 |
| HLA-C | 142840 | 216526_x_at | 5.1 +/− 0.47 | 6.7 +/− 0.29 | 1.8 +/− 0.12 | 5.0 +/− 0.35 | 1.3 +/− 0.06 |
| HLA-E | 143010 | 200904_at | 10.5 +/− 2.86 | 17.9 +/− 1.35 | 1.0 +/− 0.48 | 9.4 +/− 0.97 | 0.9 +/− 0.24 |
| HLA-E | 143010 | 200905_x_at | 3.6 +/− 0.22 | 5.0 +/− 0.22 | 1.3 +/− 0.05 | 3.4 +/− 0.11 | 1.1 +/− 0.03 |
| HLA-E | 143010 | 217456_x_at | 2.7 +/− 0.22 | 2.9 +/− 0.26 | 1.2 +/− 0.19 | 2.5 +/− 0.38 | 1.0 +/− 0.06 |
| HLA-F | 143110 | 204806_x_at | 2.1 +/− 0.06 | 3.6 +/− 0.83 | 1.3 +/− 0.13 | 2.0 +/− 0.29 | 1.1 +/− 0.09 |
| HLA-F | 143110 | 221875_x_at | 3.3 +/− 0.29 | 4.3 +/− 0.12 | 1.3 +/− 0.06 | 2.9 +/− 0.05 | 1.2 +/− 0.08 |
| HLA-G | 142871 | 210514_x_at | 2.7 +/− 0.87 | 3.3 +/− 0.62 | 1.3 +/− 0.33 | 2.8 +/− 0.96 | 1.0 +/− 0.41 |
| HLA-G | 142871 | 211528_x_at | 2.8 +/− 0.46 | 3.0 +/− 0.23 | 1.3 +/− 0.17 | 2.5 +/− 0.29 | 1.2 +/− 0.07 |
| HLA-G | 142871 | 211529_x_at | 3.6 +/− 0.45 | 4.3 +/− 0.33 | 1.3 +/− 0.14 | 3.0 +/− 0.36 | 1.0 +/− 0.15 |
| HLA-G | 142871 | 211530_x_at | 2.6 +/− 0.57 | 3.4 +/− 1.04 | 1.3 +/− 0.54 | 2.7 +/− 0.32 | 1.1 +/− 0.34 |
| HNF4G | 605966 | 232271_at | 1.1 +/− 0.39 | 2.1 +/− 0.30 | 0.6 +/− 0.17 | 1.7 +/− 0.36 | 0.8 +/− 0.19 |
| HNRPA2B1 | 600124 | 225107_at | 1.1 +/− 0.06 | 1.1 +/− 0.03 | 1.0 +/− 0.03 | 1.1 +/− 0.09 | 0.9 +/− 0.04 |
| HNRPU | 602869 | 224820_at | 0.8 +/− 0.03 | 0.7 +/− 0.05 | 0.9 +/− 0.09 | 0.9 +/− 0.09 | 1.0 +/− 0.03 |
| HOXB7 | 142962 | 216973_s_at | 1.0 +/− 0.03 | 1.0 +/− 0.04 | 1.1 +/− 0.02 | 1.0 +/− 0.01 | 1.0 +/− 0.03 |
| HOXC6 | 142972 | 206858_s_at | 0.9 +/− 0.13 | 0.6 +/− 0.06 | 1.0 +/− 0.13 | 0.8 +/− 0.10 | 0.9 +/− 0.09 |
| HPGD | 601688 | 203914_x_at | 0.9 +/− 0.07 | 0.8 +/− 0.05 | 1.0 +/− 0.06 | 0.9 +/− 0.06 | 1.0 +/− 0.08 |
| HPGD | 601688 | 211548_s_at | 0.8 +/− 0.07 | 0.7 +/− 0.09 | 0.9 +/− 0.09 | 0.8 +/− 0.07 | 0.9 +/− 0.06 |
| HR | 602302 | 241355_at | 0.9 +/− 0.10 | 0.7 +/− 0.07 | 1.0 +/− 0.09 | 0.9 +/− 0.10 | 1.0 +/− 0.07 |
| HRMT1L1 | 601961 | 221564_at | 0.8 +/− 0.11 | 0.8 +/− 0.03 | 0.9 +/− 0.04 | 0.8 +/− 0.06 | 1.0 +/− 0.08 |
| HRMT1L2 | 602950 | 206445_s_at | 1.1 +/− 0.05 | 1.2 +/− 0.07 | 1.0 +/− 0.02 | 1.0 +/− 0.05 | 1.0 +/− 0.02 |
| HSPA2 | 140560 | 211538_s_at | 0.8 +/− 0.09 | 0.7 +/− 0.05 | 1.0 +/− 0.12 | 0.9 +/− 0.09 | 0.9 +/− 0.09 |
| HSPC195 | — | 222996_s_at | 0.9 +/− 0.04 | 0.7 +/− 0.04 | 0.9 +/− 0.09 | 0.9 +/− 0.05 | 0.9 +/− 0.04 |
| HSXIAPAF1 | 606717 | 228617_at | 6.9 +/− 0.50 | 9.3 +/− 0.87 | 1.9 +/− 0.16 | 2.9 +/− 0.38 | 1.3 +/− 0.20 |
| HT021 | — | 219288_at | 1.1 +/− 0.05 | 1.3 +/− 0.10 | 1.0 +/− 0.10 | 1.1 +/− 0.12 | 1.0 +/− 0.11 |
| HTGN29 | — | 203024_s_at | 1.2 +/− 0.03 | 1.7 +/− 0.08 | 1.0 +/− 0.06 | 1.2 +/− 0.05 | 1.1 +/− 0.07 |
| HTLF | 143089 | 226711_at | 1.1 +/− 0.05 | 1.3 +/− 0.12 | 1.0 +/− 0.01 | 1.2 +/− 0.05 | 1.0 +/− 0.09 |
| IFI16 | 147586 | 206332_s_at | 2.4 +/− 0.14 | 2.9 +/− 0.30 | 2.1 +/− 0.34 | 2.0 +/− 0.42 | 1.4 +/− 0.19 |
| IFI16 | 147586 | 208966_x_at | 1.9 +/− 0.32 | 2.1 +/− 0.27 | 1.8 +/− 0.25 | 1.6 +/− 0.15 | 1.4 +/− 0.08 |
| IFI27 | 600009 | 202411_at | 19.4 +/− 3.08 | 8.5 +/− 2.41 | 40.0 +/− 4.92 | 2.3 +/− 0.98 | 8.5 +/− 3.44 |
| IFI30 | 604664 | 201422_at | 2.5 +/− 0.11 | 3.7 +/− 0.24 | 1.2 +/− 0.22 | 2.4 +/− 0.24 | 1.1 +/− 0.12 |
| IFI35 | 600735 | 209417_s_at | 8.6 +/− 0.43 | 12.3 +/− 0.63 | 4.0 +/− 0.50 | 6.5 +/− 0.51 | 2.3 +/− 0.23 |
| IFI44 | — | 214453_s_at | 6.6 +/− 0.57 | 6.8 +/− 0.92 | 5.8 +/− 1.18 | 2.9 +/− 0.87 | 2.9 +/− 0.64 |
| IFIT1 | 147690 | 203153_at | 4.1 +/− 0.34 | 2.5 +/− 0.29 | 10.7 +/− 1.53 | 1.7 +/− 0.40 | 3.8 +/− 0.59 |
| IFIT2 | 147040 | 226757_at | 2.1 +/− 0.20 | 3.1 +/− 0.57 | 1.2 +/− 0.37 | 1.7 +/− 0.29 | 1.0 +/− 0.05 |
| IFIT4 | 604650 | 204747_at | 3.3 +/− 0.13 | 5.0 +/− 0.08 | 1.9 +/− 0.07 | 2.6 +/− 0.31 | 1.3 +/− 0.28 |
| IFIT4 | 604650 | 229450_at | 8.3 +/− 0.86 | 13.5 +/− 1.33 | 3.0 +/− 0.24 | 5.4 +/− 1.00 | 1.7 +/− 0.34 |
| IFITM1 | 604456 | 201601_x_at | 8.5 +/− 1.37 | 6.7 +/− 0.57 | 6.4 +/− 0.71 | 2.3 +/− 0.43 | 2.4 +/− 0.83 |
| IFITM1 | 604456 | 214022_s_at | 9.5 +/− 0.94 | 7.1 +/− 0.63 | 7.3 +/− 1.14 | 1.9 +/− 0.38 | 2.2 +/− 0.28 |
| IFITM2 | 605578 | 201315_x_at | 2.4 +/− 0.37 | 2.8 +/− 0.58 | 2.6 +/− 0.45 | 1.8 +/− 0.26 | 1.5 +/− 0.25 |
| IFITM3 | 605579 | 212203_x_at | 7.3 +/− 0.61 | 8.0 +/− 0.23 | 4.5 +/− 0.62 | 3.9 +/− 0.53 | 2.2 +/− 0.58 |
| IFNGR1 | 107470 | 202727_s_at | 1.1 +/− 0.09 | 1.2 +/− 0.08 | 1.0 +/− 0.03 | 1.1 +/− 0.06 | 1.0 +/− 0.06 |
| IL15RA | 601070 | 207375_s_at | 5.6 +/− 0.35 | 8.7 +/− 0.58 | 0.6 +/− 0.19 | 5.6 +/− 0.49 | 1.3 +/− 0.60 |
| IL18BP | 604113 | 222868_s_at | 12.6 +/− 1.62 | 32.0 +/− 2.94 | 1.0 +/− 0.27 | 14.8 +/− 1.18 | 1.3 +/− 0.47 |
| IL6R | 147880 | 205945_at | 1.3 +/− 0.22 | 1.7 +/− 0.22 | 1.0 +/− 0.07 | 1.3 +/− 0.15 | 0.8 +/− 0.19 |
| IL6ST | 600694 | 212195_at | 1.1 +/− 0.02 | 1.1 +/− 0.04 | 1.0 +/− 0.03 | 1.1 +/− 0.07 | 1.0 +/− 0.02 |
| IL7 | 146660 | 206693_at | 3.8 +/− 0.47 | 9.8 +/− 1.78 | 3.4 +/− 2.26 | 3.4 +/− 0.44 | 3.1 +/− 0.79 |
| INADL | 603199 | 223681_s_at | 0.8 +/− 0.07 | 0.7 +/− 0.06 | 0.9 +/− 0.07 | 0.8 +/− 0.11 | 1.0 +/− 0.07 |
| INDO | 147435 | 210029_at | 7.0 +/− 1.38 | 37.6 +/− 4.64 | 0.8 +/− 0.61 | 7.1 +/− 1.07 | 0.5 +/− 0.17 |
| INSIG2 | — | 209566_at | 0.9 +/− 0.06 | 0.9 +/− 0.07 | 1.0 +/− 0.07 | 0.9 +/− 0.04 | 1.1 +/− 0.05 |
| IRF1 | 147575 | 202531_at | 3.1 +/− 0.12 | 7.5 +/− 0.36 | 1.1 +/− 0.06 | 3.3 +/− 0.21 | 1.1 +/− 0.09 |
| IRF2 | 147576 | 203275_at | 1.3 +/− 0.11 | 1.7 +/− 0.23 | 1.0 +/− 0.10 | 1.3 +/− 0.07 | 1.1 +/− 0.11 |
| IRF7 | 605047 | 208436_s_at | 2.7 +/− 0.39 | 1.5 +/− 0.12 | 4.1 +/− 0.36 | 1.4 +/− 0.19 | 2.8 +/− 0.68 |
| ISG20 | 604533 | 204698_at | 6.4 +/− 0.95 | 11.4 +/− 0.90 | 1.1 +/− 0.76 | 3.6 +/− 0.85 | 0.8 +/− 1.06 |
| ISG20 | 604533 | 33304_at | 2.2 +/− 0.17 | 3.4 +/− 0.27 | 1.3 +/− 0.16 | 1.8 +/− 0.21 | 1.0 +/− 0.28 |
| ISGF3G | 147574 | 203882_at | 5.2 +/− 0.57 | 4.2 +/− 0.10 | 5.1 +/− 0.32 | 3.7 +/− 0.22 | 3.6 +/− 0.27 |
| JUP | 173325 | 201015_s_at | 0.7 +/− 0.09 | 0.5 +/− 0.03 | 0.9 +/− 0.07 | 0.7 +/− 0.11 | 0.9 +/− 0.02 |
| KARS | 601421 | 200079_s_at | 1.2 +/− 0.08 | 1.3 +/− 0.07 | 1.1 +/− 0.10 | 1.2 +/− 0.11 | 1.1 +/− 0.10 |
| KIAA0545 | — | 213600_at | 0.9 +/− 0.03 | 0.8 +/− 0.03 | 1.0 +/− 0.10 | 0.9 +/− 0.06 | 1.0 +/− 0.07 |
| KIAA0650 | — | 212569_at | 1.0 +/− 0.09 | 1.3 +/− 0.08 | 0.9 +/− 0.17 | 1.2 +/− 0.15 | 0.9 +/− 0.15 |
| KIAA0650 | — | 212579_at | 1.0 +/− 0.08 | 1.2 +/− 0.07 | 0.9 +/− 0.05 | 1.1 +/− 0.04 | 1.0 +/− 0.12 |
| KIAA0650 | — | 241620_at | 1.5 +/− 0.37 | 1.8 +/− 0.22 | 0.9 +/− 0.26 | 1.3 +/− 0.17 | 1.1 +/− 0.13 |
| KIAA0746 | — | 212314_at | 0.8 +/− 0.04 | 0.7 +/− 0.06 | 0.9 +/− 0.10 | 0.8 +/− 0.12 | 0.9 +/− 0.06 |
| KIAA0882 | — | 212956_at | 0.9 +/− 0.11 | 0.8 +/− 0.01 | 1.1 +/− 0.08 | 0.9 +/− 0.10 | 1.1 +/− 0.08 |
| KIAA0992 | — | 200897_s_at | 0.8 +/− 0.08 | 0.7 +/− 0.03 | 1.0 +/− 0.10 | 0.8 +/− 0.09 | 0.9 +/− 0.08 |
| KIAA0992 | — | 200907_s_at | 0.7 +/− 0.06 | 0.6 +/− 0.04 | 0.9 +/− 0.04 | 0.7 +/− 0.08 | 0.9 +/− 0.09 |
| KIAA1268 | — | 224701_at | 4.8 +/− 0.46 | 7.2 +/− 0.89 | 1.9 +/− 0.17 | 3.7 +/− 0.43 | 1.6 +/− 0.15 |
| KIAA1295 | — | 231823_s_at | 0.9 +/− 0.11 | 0.7 +/− 0.10 | 1.1 +/− 0.12 | 0.8 +/− 0.07 | 0.9 +/− 0.08 |
| KIAA1340 | — | 225732_at | 0.9 +/− 0.03 | 0.9 +/− 0.03 | 0.9 +/− 0.02 | 0.9 +/− 0.05 | 0.9 +/− 0.03 |
| KIAA1345 | — | 234936_s_at | 0.8 +/− 0.09 | 0.7 +/− 0.07 | 1.0 +/− 0.05 | 0.9 +/− 0.10 | 1.0 +/− 0.06 |
| KIAA1414 | — | 233642_s_at | 0.9 +/− 0.07 | 0.7 +/− 0.06 | 0.9 +/− 0.07 | 0.9 +/− 0.09 | 0.8 +/− 0.12 |
| KIAA1554 | — | 225929_s_at | 1.4 +/− 0.31 | 2.2 +/− 0.44 | 1.1 +/− 0.20 | 1.3 +/− 0.17 | 0.9 +/− 0.10 |
| KIAA1554 | — | 225931_s_at | 1.4 +/− 0.15 | 1.9 +/− 0.33 | 1.1 +/− 0.16 | 1.3 +/− 0.14 | 1.0 +/− 0.11 |
| KIAA1618 | — | 231956_at | 1.3 +/− 0.06 | 1.8 +/− 0.22 | 1.0 +/− 0.13 | 1.4 +/− 0.29 | 1.1 +/− 0.15 |
| KIAA1618 | — | 232155_at | 1.4 +/− 0.29 | 2.2 +/− 0.40 | 1.1 +/− 0.52 | 1.1 +/− 0.26 | 0.6 +/− 0.32 |
| KIAA1618 | — | 241347_at | 1.6 +/− 0.37 | 2.6 +/− 0.64 | 1.0 +/− 0.14 | 1.3 +/− 0.40 | 1.1 +/− 0.26 |
| KIAA1685 | — | 218659_at | 1.0 +/− 0.04 | 1.1 +/− 0.03 | 1.0 +/− 0.04 | 1.1 +/− 0.06 | 1.0 +/− 0.02 |
| KIAA1706 | — | 225630_at | 0.7 +/− 0.10 | 0.7 +/− 0.05 | 1.1 +/− 0.16 | 0.7 +/− 0.16 | 0.9 +/− 0.12 |
| KIAA1724 | — | 1555274_a_at | 1.1 +/− 0.05 | 1.3 +/− 0.06 | 1.0 +/− 0.08 | 1.2 +/− 0.10 | 1.0 +/− 0.03 |
| KIAA1935 | — | 228336_at | 1.0 +/− 0.08 | 0.9 +/− 0.07 | 1.0 +/− 0.07 | 0.8 +/− 0.02 | 1.0 +/− 0.03 |
| KIAA1946 | — | 227370_at | 0.8 +/− 0.16 | 0.6 +/− 0.09 | 1.0 +/− 0.15 | 0.8 +/− 0.04 | 1.1 +/− 0.10 |
| KIP2 | 605564 | 205008_s_at | 0.8 +/− 0.06 | 0.7 +/− 0.08 | 1.0 +/− 0.05 | 0.8 +/− 0.11 | 0.9 +/− 0.05 |
| KLF12 | 607531 | 227261_at | 0.7 +/− 0.08 | 0.7 +/− 0.05 | 0.9 +/− 0.15 | 0.7 +/− 0.07 | 0.9 +/− 0.19 |
| KLHL5 | — | 232297_at | 0.8 +/− 0.05 | 0.8 +/− 0.04 | 0.9 +/− 0.03 | 0.9 +/− 0.06 | 1.0 +/− 0.08 |
| KPNA1 | 600686 | 202055_at | 0.9 +/− 0.03 | 0.9 +/− 0.06 | 1.0 +/− 0.02 | 1.0 +/− 0.02 | 0.9 +/− 0.03 |
| KRT19 | 148020 | 201650_at | 0.7 +/− 0.06 | 0.4 +/− 0.07 | 1.0 +/− 0.12 | 0.6 +/− 0.08 | 1.0 +/− 0.10 |
| LAP3 | 170250 | 217933_s_at | 4.1 +/− 0.31 | 6.8 +/− 0.32 | 2.3 +/− 0.23 | 3.7 +/− 0.18 | 1.7 +/− 0.13 |
| LEPR | 601007 | 202377_at | 0.9 +/− 0.03 | 0.8 +/− 0.07 | 1.0 +/− 0.03 | 0.9 +/− 0.05 | 1.0 +/− 0.03 |
| LGALS3BP | 600626 | 200923_at | 2.6 +/− 0.14 | 2.7 +/− 0.36 | 2.0 +/− 0.17 | 2.5 +/− 0.21 | 1.4 +/− 0.05 |
| LL5beta | — | 225688_s_at | 1.0 +/− 0.12 | 1.3 +/− 0.10 | 1.0 +/− 0.13 | 1.1 +/− 0.11 | 0.9 +/− 0.06 |
| LMCD1 | 604859 | 218574_s_at | 0.8 +/− 0.06 | 0.7 +/− 0.02 | 1.0 +/− 0.09 | 0.9 +/− 0.13 | 1.0 +/− 0.07 |
| LOC116071 | — | 228439_at | 5.9 +/− 0.61 | 13.6 +/− 1.78 | 2.3 +/− 0.38 | 5.2 +/− 0.43 | 1.1 +/− 0.56 |
| LOC116228 | — | 225786_at | 1.1 +/− 0.13 | 1.3 +/− 0.07 | 0.9 +/− 0.15 | 1.2 +/− 0.16 | 0.8 +/− 0.13 |
| LOC119504 | — | 224664_at | 0.9 +/− 0.04 | 0.9 +/− 0.04 | 1.0 +/− 0.05 | 1.0 +/− 0.03 | 1.0 +/− 0.02 |
| LOC129607 | — | 226702_at | 3.1 +/− 0.17 | 3.5 +/− 0.27 | 2.1 +/− 0.72 | 2.1 +/− 0.34 | 1.3 +/− 0.38 |
| LOC129642 | — | 226726_at | 0.8 +/− 0.12 | 0.7 +/− 0.06 | 0.9 +/− 0.08 | 0.8 +/− 0.11 | 0.8 +/− 0.06 |
| LOC144501 | — | 231849_at | 0.7 +/− 0.14 | 0.5 +/− 0.04 | 1.0 +/− 0.15 | 0.7 +/− 0.11 | 0.9 +/− 0.18 |
| LOC151636 | — | 225415_at | 2.9 +/− 0.13 | 3.9 +/− 0.09 | 2.0 +/− 0.07 | 2.5 +/− 0.30 | 1.5 +/− 0.15 |
| LOC157378 | — | 241342_at | 0.9 +/− 0.04 | 0.8 +/− 0.01 | 0.9 +/− 0.05 | 0.9 +/− 0.09 | 1.0 +/− 0.04 |
| LOC159090 | — | 222673_x_at | 0.8 +/− 0.04 | 0.8 +/− 0.05 | 0.9 +/− 0.06 | 0.9 +/− 0.06 | 0.9 +/− 0.04 |
| LOC169611 | — | 213075_at | 0.6 +/− 0.33 | 0.2 +/− 0.05 | 1.3 +/− 0.27 | 0.6 +/− 0.20 | 1.0 +/− 0.20 |
| LOC221002 | — | 230563_at | 0.7 +/− 0.11 | 0.6 +/− 0.07 | 0.9 +/− 0.06 | 0.8 +/− 0.10 | 0.9 +/− 0.06 |
| LOC221061 | — | 212771_at | 0.8 +/− 0.11 | 0.7 +/− 0.14 | 1.0 +/− 0.09 | 0.8 +/− 0.10 | 1.1 +/− 0.08 |
| LOC253827 | — | 225782_at | 0.8 +/− 0.04 | 0.6 +/− 0.07 | 0.9 +/− 0.11 | 0.8 +/− 0.04 | 0.9 +/− 0.05 |
| LOC255743 | — | 225911_at | 0.8 +/− 0.06 | 0.7 +/− 0.09 | 1.0 +/− 0.07 | 0.8 +/− 0.12 | 1.0 +/− 0.05 |
| LOC285812 | — | 230179_at | 1.2 +/− 0.10 | 1.3 +/− 0.03 | 1.0 +/− 0.05 | 1.3 +/− 0.17 | 1.0 +/− 0.06 |
| LOC348531 | — | 227517_s_at | 1.1 +/− 0.04 | 1.2 +/− 0.05 | 1.0 +/− 0.05 | 1.1 +/− 0.06 | 1.0 +/− 0.04 |
| LOC51064 | — | 217751_at | 1.4 +/− 0.17 | 1.6 +/− 0.06 | 0.9 +/− 0.09 | 1.3 +/− 0.01 | 0.9 +/− 0.08 |
| LOC51255 | — | 223064_at | 1.1 +/− 0.08 | 1.3 +/− 0.07 | 1.0 +/− 0.03 | 1.1 +/− 0.06 | 1.0 +/− 0.06 |
| LOC51315 | — | 218303_x_at | 0.9 +/− 0.05 | 0.8 +/− 0.05 | 1.0 +/− 0.06 | 0.9 +/− 0.06 | 1.0 +/− 0.03 |
| LOC54103 | — | 213142_x_at | 1.3 +/− 0.13 | 1.7 +/− 0.13 | 1.0 +/− 0.09 | 1.4 +/− 0.18 | 1.0 +/− 0.10 |
| LOC54103 | — | 222150_s_at | 1.3 +/− 0.11 | 1.7 +/− 0.14 | 1.1 +/− 0.11 | 1.4 +/− 0.16 | 1.1 +/− 0.11 |
| LOC84549 | — | 211686_s_at | 1.0 +/− 0.04 | 1.2 +/− 0.05 | 1.0 +/− 0.06 | 1.1 +/− 0.06 | 1.0 +/− 0.07 |
| LOC85026 | — | 226405_s_at | 0.9 +/− 0.06 | 0.7 +/− 0.08 | 0.9 +/− 0.10 | 0.8 +/− 0.08 | 0.9 +/− 0.09 |
| LOC90637 | — | 226650_at | 1.0 +/− 0.08 | 1.2 +/− 0.07 | 0.9 +/− 0.04 | 1.0 +/− 0.09 | 0.9 +/− 0.07 |
| LOC92689 | — | 213455_at | 0.9 +/− 0.05 | 0.8 +/− 0.04 | 1.0 +/− 0.09 | 0.9 +/− 0.06 | 1.0 +/− 0.06 |
| LOC93349 | — | 214791_at | 1.8 +/− 0.21 | 2.2 +/− 0.22 | 1.0 +/− 0.18 | 1.7 +/− 0.51 | 1.0 +/− 0.22 |
| LRAP | — | 227462_at | 3.9 +/− 1.00 | 6.5 +/− 0.38 | 1.0 +/− 0.57 | 4.3 +/− 1.27 | 1.3 +/− 0.30 |
| LRAP | — | 235104_at | 3.1 +/− 1.05 | 4.7 +/− 0.99 | 1.4 +/− 0.15 | 3.0 +/− 0.58 | 0.9 +/− 0.27 |
| LSM6 | 607286 | 205036_at | 1.2 +/− 0.10 | 1.5 +/− 0.16 | 1.1 +/− 0.06 | 1.3 +/− 0.15 | 1.1 +/− 0.04 |
| LTBP1 | 150390 | 202729_s_at | 0.7 +/− 0.14 | 0.6 +/− 0.06 | 0.9 +/− 0.10 | 0.7 +/− 0.12 | 0.8 +/− 0.09 |
| LXN | — | 218729_at | 1.5 +/− 0.26 | 2.7 +/− 0.40 | 1.1 +/− 0.29 | 1.6 +/− 0.19 | 1.1 +/− 0.17 |
| MAL2 | — | 224650_at | 1.3 +/− 0.23 | 1.6 +/− 0.26 | 0.8 +/− 0.13 | 1.5 +/− 0.37 | 0.9 +/− 0.14 |
| MAP2K1IP1 | 603296 | 227562_at | 0.7 +/− 0.09 | 0.5 +/− 0.25 | 0.8 +/− 0.11 | 0.6 +/− 0.11 | 0.9 +/− 0.18 |
| MARCKS | 177061 | 201670_s_at | 1.1 +/− 0.10 | 0.7 +/− 0.08 | 1.0 +/− 0.05 | 0.9 +/− 0.09 | 1.1 +/− 0.13 |
| MARCKS | 177061 | 225897_at | 0.8 +/− 0.03 | 0.7 +/− 0.03 | 1.0 +/− 0.01 | 0.9 +/− 0.11 | 1.0 +/− 0.02 |
| MATN3 | 602109 | 206091_at | 0.7 +/− 0.09 | 0.6 +/− 0.19 | 1.0 +/− 0.08 | 0.9 +/− 0.19 | 1.1 +/− 0.15 |
| MAX | 154950 | 209332_s_at | 1.1 +/− 0.09 | 1.3 +/− 0.12 | 1.0 +/− 0.07 | 1.1 +/− 0.09 | 1.0 +/− 0.07 |
| MB | 160000 | 204179_at | 0.6 +/− 0.20 | 0.4 +/− 0.10 | 1.0 +/− 0.16 | 0.5 +/− 0.07 | 1.0 +/− 0.16 |
| MCAM | 155735 | 211340_s_at | 0.8 +/− 0.02 | 0.7 +/− 0.06 | 1.0 +/− 0.04 | 0.8 +/− 0.09 | 1.0 +/− 0.13 |
| MDA5 | 606951 | 219209_at | 33.2 +/− 6.25 | 48.4 +/− 4.43 | 26.5 +/− 5.78 | 21.4 +/− 1.74 | 9.8 +/− 4.25 |
| MDK | 162096 | 209035_at | 1.5 +/− 0.22 | 1.8 +/− 0.11 | 0.9 +/− 0.27 | 1.3 +/− 0.23 | 1.0 +/− 0.16 |
| ME2 | 154270 | 209397_at | 1.1 +/− 0.06 | 1.1 +/− 0.06 | 1.0 +/− 0.07 | 1.1 +/− 0.02 | 1.0 +/− 0.03 |
| MET | 164860 | 203510_at | 1.2 +/− 0.15 | 1.3 +/− 0.07 | 1.0 +/− 0.10 | 1.1 +/− 0.06 | 1.0 +/− 0.10 |
| METTL2 | 607846 | 221079_s_at | 1.1 +/− 0.08 | 1.1 +/− 0.16 | 1.1 +/− 0.14 | 1.0 +/− 0.07 | 1.4 +/− 0.16 |
| MGC12981 | — | 224946_s_at | 1.0 +/− 0.08 | 0.9 +/− 0.03 | 1.0 +/− 0.06 | 1.0 +/− 0.02 | 1.0 +/− 0.03 |
| MGC15397 | — | 225991_at | 1.0 +/− 0.12 | 1.2 +/− 0.10 | 0.9 +/− 0.08 | 1.1 +/− 0.08 | 1.0 +/− 0.05 |
| MGC15397 | — | 235037_at | 1.2 +/− 0.05 | 1.3 +/− 0.06 | 1.0 +/− 0.09 | 1.1 +/− 0.16 | 0.9 +/− 0.10 |
| MGC16044 | — | 228298_at | 0.9 +/− 0.04 | 0.7 +/− 0.07 | 1.1 +/− 0.18 | 0.9 +/− 0.08 | 1.0 +/− 0.08 |
| MGC16202 | — | 226326_at | 1.0 +/− 0.06 | 1.1 +/− 0.03 | 0.9 +/− 0.07 | 1.0 +/− 0.05 | 0.9 +/− 0.03 |
| MGC16202 | — | 227935_s_at | 1.3 +/− 0.17 | 1.5 +/− 0.16 | 1.1 +/− 0.13 | 1.3 +/− 0.13 | 1.0 +/− 0.15 |
| MGC20553 | 607619 | 230645_at | 1.0 +/− 0.03 | 1.2 +/− 0.12 | 0.9 +/− 0.03 | 1.0 +/− 0.10 | 0.9 +/− 0.05 |
| MGC22805 | — | 238439_at | 1.6 +/− 0.13 | 2.3 +/− 0.23 | 0.9 +/− 0.11 | 1.8 +/− 0.29 | 1.1 +/− 0.13 |
| MGC23427 | — | 212993_at | 0.9 +/− 0.06 | 0.8 +/− 0.04 | 0.8 +/− 0.08 | 0.9 +/− 0.02 | 0.9 +/− 0.03 |
| MGC3295 | — | 219569_s_at | 1.2 +/− 0.08 | 1.4 +/− 0.09 | 1.1 +/− 0.12 | 1.2 +/− 0.11 | 1.0 +/− 0.05 |
| MGC33602 | — | 225579_at | 0.9 +/− 0.04 | 0.8 +/− 0.08 | 1.0 +/− 0.06 | 0.9 +/− 0.09 | 1.0 +/− 0.07 |
| MGC35274 | — | 226748_at | 1.1 +/− 0.10 | 1.3 +/− 0.14 | 1.0 +/− 0.08 | 1.2 +/− 0.11 | 1.0 +/− 0.07 |
| MGC39350 | — | 228700_at | 1.2 +/− 0.28 | 1.7 +/− 0.13 | 0.9 +/− 0.43 | 1.4 +/− 0.39 | 0.8 +/− 0.09 |
| MGC4054 | — | 220444_at | 1.6 +/− 0.23 | 1.1 +/− 0.10 | 1.6 +/− 0.34 | 1.5 +/− 0.22 | 1.6 +/− 0.14 |
| MGC4309 | — | 219476_at | 0.5 +/− 0.21 | 0.4 +/− 0.15 | 1.0 +/− 0.07 | 0.5 +/− 0.12 | 0.9 +/− 0.28 |
| MGC45871 | — | 226876_at | 0.8 +/− 0.07 | 0.7 +/− 0.03 | 0.9 +/− 0.07 | 0.8 +/− 0.08 | 0.9 +/− 0.05 |
| MGC45871 | — | 226905_at | 0.7 +/− 0.10 | 0.6 +/− 0.03 | 0.8 +/− 0.11 | 0.7 +/− 0.08 | 0.9 +/− 0.06 |
| MGC4707 | — | 203257_s_at | 1.0 +/− 0.08 | 0.8 +/− 0.07 | 1.1 +/− 0.09 | 0.9 +/− 0.14 | 1.1 +/− 0.09 |
| MGC49942 | — | 224573_at | 1.1 +/− 0.03 | 1.1 +/− 0.03 | 1.1 +/− 0.04 | 0.9 +/− 0.03 | 1.0 +/− 0.06 |
| MGLL | — | 211026_s_at | 1.0 +/− 0.06 | 0.9 +/− 0.03 | 1.1 +/− 0.04 | 0.9 +/− 0.04 | 1.0 +/− 0.06 |
| MGST1 | 138330 | 239001_at | 1.3 +/− 0.10 | 1.2 +/− 0.07 | 1.0 +/− 0.08 | 1.1 +/− 0.06 | 0.9 +/− 0.11 |
| MICA | 600169 | 205904_at | 0.7 +/− 0.08 | 0.5 +/− 0.02 | 1.0 +/− 0.07 | 0.6 +/− 0.03 | 1.0 +/− 0.10 |
| MICA | 600169 | 205905_s_at | 0.8 +/− 0.10 | 0.7 +/− 0.08 | 1.1 +/− 0.08 | 0.8 +/− 0.13 | 0.9 +/− 0.06 |
| MICB | 602436 | 206247_at | 1.2 +/− 0.19 | 1.5 +/− 0.12 | 0.9 +/− 0.15 | 1.3 +/− 0.18 | 0.9 +/− 0.14 |
| MIPOL1 | 606850 | 244246_at | 0.9 +/− 0.20 | 1.0 +/− 0.08 | 0.9 +/− 0.16 | 0.9 +/− 0.16 | 0.5 +/− 0.14 |
| MITF | 156845 | 207233_s_at | 0.9 +/− 0.07 | 0.8 +/− 0.03 | 1.0 +/− 0.07 | 0.9 +/− 0.04 | 1.0 +/− 0.05 |
| MMD | 604467 | 203414_at | 1.0 +/− 0.05 | 1.1 +/− 0.05 | 0.8 +/− 0.06 | 1.0 +/− 0.08 | 0.8 +/− 0.07 |
| MMP24 | 604871 | 225860_at | 0.9 +/− 0.07 | 0.8 +/− 0.05 | 1.0 +/− 0.11 | 0.8 +/− 0.05 | 1.0 +/− 0.04 |
| MOV10 | — | 223849_s_at | 1.5 +/− 0.15 | 1.6 +/− 0.04 | 1.1 +/− 0.16 | 1.5 +/− 0.18 | 1.1 +/− 0.14 |
| MPDZ | 603785 | 205079_s_at | 0.8 +/− 0.07 | 0.7 +/− 0.15 | 0.9 +/− 0.11 | 0.8 +/− 0.03 | 1.0 +/− 0.05 |
| MPRG | 607781 | 242871_at | 1.1 +/− 0.04 | 1.2 +/− 0.06 | 1.0 +/− 0.05 | 1.2 +/− 0.06 | 1.0 +/− 0.05 |
| MRCL3 | — | 201318_s_at | 1.1 +/− 0.05 | 1.2 +/− 0.05 | 1.1 +/− 0.09 | 1.0 +/− 0.07 | 1.1 +/− 0.04 |
| MRS3/4/// | — | 221432_s_at | 1.2 +/− 0.16 | 1.4 +/− 0.11 | 0.9 +/− 0.15 | 1.1 +/− 0.14 | 1.0 +/− 0.06 |
| [symbol] | | | | | | | |
| MT1G | 156353 | 204745_x_at | 1.2 +/− 0.12 | 1.7 +/− 0.22 | 1.2 +/− 0.13 | 1.2 +/− 0.12 | 1.0 +/− 0.19 |
| MT2A | 156360 | 212185_x_at | 1.4 +/− 0.13 | 1.7 +/− 0.14 | 1.1 +/− 0.22 | 1.2 +/− 0.08 | 1.1 +/− 0.26 |
| MTIF2 | 603766 | 203095_at | 1.1 +/− 0.04 | 1.2 +/− 0.06 | 0.9 +/− 0.06 | 1.1 +/− 0.04 | 0.9 +/− 0.02 |
| MUC1 | 158340 | 213693_s_at | 1.7 +/− 0.26 | 2.2 +/− 0.10 | 1.1 +/− 0.41 | 1.9 +/− 0.32 | 0.7 +/− 0.26 |
| MX1 | 147150 | 202086_at | 4.7 +/− 0.95 | 4.0 +/− 1.12 | 7.3 +/− 1.62 | 2.3 +/− 0.34 | 3.2 +/− 0.44 |
| MYD88 | 602170 | 209124_at | 1.3 +/− 0.09 | 1.4 +/− 0.09 | 1.3 +/− 0.07 | 1.3 +/− 0.06 | 1.1 +/− 0.07 |
| MYH10 | 160776 | 212372_at | 0.8 +/− 0.13 | 0.7 +/− 0.04 | 0.9 +/− 0.11 | 0.8 +/− 0.07 | 1.0 +/− 0.06 |
| MYO5A | 160777 | 227761_at | 0.8 +/− 0.09 | 0.7 +/− 0.03 | 0.9 +/− 0.10 | 0.9 +/− 0.07 | 0.9 +/− 0.10 |
| MYO5B | 606540 | 225301_s_at | 0.7 +/− 0.08 | 0.7 +/− 0.08 | 1.0 +/− 0.13 | 0.8 +/− 0.07 | 1.0 +/− 0.14 |
| MYO5C | — | 218966_at | 0.8 +/− 0.06 | 0.7 +/− 0.06 | 1.0 +/− 0.11 | 0.8 +/− 0.06 | 1.0 +/− 0.17 |
| na | — | 226245_at | 0.9 +/− 0.09 | 0.8 +/− 0.03 | 1.0 +/− 0.02 | 0.8 +/− 0.07 | 1.0 +/− 0.05 |
| na | — | 234987_at | 3.1 +/− 0.53 | 3.1 +/− 0.39 | 2.2 +/− 0.42 | 1.8 +/− 0.23 | 1.4 +/− 0.19 |
| na | — | 235229_at | 1.8 +/− 0.40 | 3.5 +/− 0.88 | 1.0 +/− 0.47 | 2.3 +/− 0.69 | 1.4 +/− 0.17 |
| na | — | 235529_x_at | 2.4 +/− 0.20 | 2.7 +/− 0.28 | 2.0 +/− 0.32 | 1.7 +/− 0.37 | 1.1 +/− 0.13 |
| na | — | 241353_s_at | 0.9 +/− 0.14 | 0.8 +/− 0.06 | 0.9 +/− 0.04 | 0.8 +/− 0.06 | 1.0 +/− 0.07 |
| NAP1L1 | 164060 | 208752_x_at | 1.1 +/− 0.05 | 1.1 +/− 0.04 | 1.0 +/− 0.04 | 1.0 +/− 0.02 | 1.0 +/− 0.01 |
| NAV2 | 607026 | 218330_s_at | 0.8 +/− 0.08 | 0.6 +/− 0.08 | 0.9 +/− 0.11 | 0.8 +/− 0.03 | 0.9 +/− 0.04 |
| NBS1 | 602667 | 202906_s_at | 1.2 +/− 0.15 | 1.4 +/− 0.16 | 1.0 +/− 0.11 | 1.3 +/− 0.14 | 1.0 +/− 0.05 |
| NBS1 | 602667 | 202907_s_at | 1.1 +/− 0.05 | 1.3 +/− 0.02 | 1.0 +/− 0.08 | 1.1 +/− 0.07 | 1.0 +/− 0.02 |
| NCOA7 | — | 225344_at | 1.1 +/− 0.07 | 1.3 +/− 0.03 | 1.1 +/− 0.07 | 1.1 +/− 0.07 | 1.0 +/− 0.05 |
| NEK7 | 606848 | 212530_at | 1.0 +/− 0.05 | 1.2 +/− 0.01 | 1.0 +/− 0.02 | 1.1 +/− 0.09 | 1.0 +/− 0.04 |
| NFE2L3 | 604135 | 204702_s_at | 1.4 +/− 0.25 | 2.3 +/− 0.63 | 1.0 +/− 0.23 | 1.7 +/− 0.54 | 0.8 +/− 0.18 |
| NFE2L3 | 604135 | 236471_at | 1.7 +/− 0.15 | 2.5 +/− 0.27 | 1.2 +/− 0.30 | 1.8 +/− 0.57 | 1.1 +/− 0.07 |
| NFIX | 164005 | 227400_at | 2.6 +/− 0.50 | 3.3 +/− 0.76 | 0.9 +/− 0.20 | 2.4 +/− 0.31 | 0.6 +/− 0.26 |
| NK4 | 606001 | 203828_s_at | 45.0 +/− 13.40 | 71.6 +/− 18.54 | 0.9 +/− 0.23 | 40.5 +/− 8.85 | 0.8 +/− 0.17 |
| NKX3-1 | 602041 | 209706_at | 1.3 +/− 0.06 | 1.4 +/− 0.08 | 1.0 +/− 0.06 | 1.2 +/− 0.13 | 1.1 +/− 0.10 |
| NMES1 | — | 223484_at | 2.1 +/− 0.43 | 3.0 +/− 0.51 | 1.1 +/− 0.29 | 2.1 +/− 0.55 | 1.0 +/− 0.24 |
| NMI | 603525 | 203964_at | 2.7 +/− 0.24 | 3.9 +/− 0.36 | 1.2 +/− 0.07 | 2.6 +/− 0.37 | 1.1 +/− 0.02 |
| NNMT | 600008 | 202237_at | 1.2 +/− 0.14 | 1.6 +/− 0.06 | 1.0 +/− 0.12 | 1.2 +/− 0.12 | 1.0 +/− 0.11 |
| NNMT | 600008 | 202238_s_at | 1.3 +/− 0.13 | 1.7 +/− 0.11 | 1.0 +/− 0.20 | 1.2 +/− 0.10 | 1.1 +/− 0.24 |
| NOD27 | — | 226474_at | 3.9 +/− 0.27 | 6.0 +/− 1.02 | 1.1 +/− 0.18 | 3.4 +/− 0.43 | 1.1 +/− 0.18 |
| NOLA1 | 606468 | 219110_at | 1.1 +/− 0.03 | 1.2 +/− 0.05 | 1.0 +/− 0.08 | 1.0 +/− 0.05 | 1.1 +/− 0.07 |
| NOP5/NOP5 | — | 223096_at | 1.1 +/− 0.06 | 1.2 +/− 0.01 | 1.1 +/− 0.05 | 1.1 +/− 0.05 | 1.0 +/− 0.07 |
| [symbol] | | | | | | | |
| NPC2 | 601015 | 200701_at | 1.1 +/− 0.05 | 1.2 +/− 0.05 | 1.0 +/− 0.06 | 1.0 +/− 0.04 | 1.0 +/− 0.04 |
| NT5E | 129190 | 203939_at | 1.5 +/− 0.25 | 1.8 +/− 0.26 | 1.2 +/− 0.15 | 1.6 +/− 0.27 | 1.2 +/− 0.12 |
| NUMA1 | 164009 | 235539_at | 1.2 +/− 0.14 | 2.2 +/− 0.11 | 0.9 +/− 0.31 | 1.1 +/− 0.29 | 1.0 +/− 0.38 |
| NYREN18 | 607981 | 1569030_s_at | 1.1 +/− 0.05 | 1.3 +/− 0.22 | 1.0 +/− 0.12 | 1.2 +/− 0.06 | 0.8 +/− 0.16 |
| NYREN18 | 607981 | 222512_at | 1.4 +/− 0.20 | 1.8 +/− 0.28 | 1.0 +/− 0.13 | 1.4 +/− 0.18 | 1.0 +/− 0.11 |
| OAS1 | 164350 | 202869_at | 2.1 +/− 0.21 | 2.0 +/− 0.25 | 2.6 +/− 0.20 | 1.4 +/− 0.18 | 1.8 +/− 0.14 |
| OAS1 | 164350 | 205552_s_at | 1.9 +/− 0.15 | 1.9 +/− 0.21 | 2.4 +/− 0.27 | 1.4 +/− 0.19 | 1.8 +/− 0.04 |
| OAS2 | 603350 | 204972_at | 2.7 +/− 0.59 | 2.9 +/− 0.37 | 1.6 +/− 0.46 | 1.4 +/− 0.15 | 1.6 +/− 0.39 |
| OAS3 | 603351 | 218400_at | 2.3 +/− 0.13 | 2.3 +/− 0.25 | 2.4 +/− 0.17 | 1.7 +/− 0.13 | 1.7 +/− 0.16 |
| OASL | 603281 | 210797_s_at | 1.9 +/− 0.16 | 2.2 +/− 0.14 | 1.4 +/− 0.14 | 1.5 +/− 0.18 | 1.2 +/− 0.12 |
| OCLN | 602876 | 227492_at | 0.8 +/− 0.06 | 0.7 +/− 0.11 | 1.0 +/− 0.08 | 0.7 +/− 0.06 | 1.0 +/− 0.08 |
| ODC1 | 165640 | 200790_at | 0.8 +/− 0.02 | 0.8 +/− 0.03 | 1.0 +/− 0.03 | 0.9 +/− 0.04 | 0.9 +/− 0.03 |
| OGFR | 606459 | 202841_x_at | 1.7 +/− 0.23 | 2.2 +/− 0.10 | 1.3 +/− 0.10 | 1.7 +/− 0.32 | 1.3 +/− 0.08 |
| OGFR | 606459 | 210443_x_at | 1.5 +/− 0.12 | 1.8 +/− 0.08 | 1.2 +/− 0.07 | 1.3 +/− 0.23 | 1.1 +/− 0.07 |
| OS-9 | — | 200714_x_at | 0.8 +/− 0.08 | 0.8 +/− 0.07 | 1.0 +/− 0.08 | 0.9 +/− 0.09 | 1.0 +/− 0.07 |
| OXTR | 167055 | 206825_at | 0.7 +/− 0.10 | 0.7 +/− 0.06 | 0.9 +/− 0.07 | 0.7 +/− 0.08 | 1.0 +/− 0.11 |
| P114-RHO- | — | 213039_at | 0.8 +/− 0.09 | 0.7 +/− 0.14 | 1.1 +/− 0.08 | 0.8 +/− 0.13 | 1.0 +/− 0.09 |
| [symbol] | | | | | | | |
| PAK1 | 602590 | 230100_x_at | 0.8 +/− 0.07 | 0.8 +/− 0.08 | 1.0 +/− 0.09 | 0.9 +/− 0.05 | 1.2 +/− 0.19 |
| PANX1 | — | 227107_at | 1.1 +/− 0.08 | 1.2 +/− 0.05 | 1.0 +/− 0.07 | 1.1 +/− 0.03 | 1.0 +/− 0.10 |
| PBEF | — | 1555167_s_at | 1.6 +/− 0.37 | 2.3 +/− 0.38 | 1.1 +/− 0.44 | 1.6 +/− 0.45 | 1.2 +/− 0.26 |
| PBEF | — | 217738_at | 1.2 +/− 0.07 | 1.7 +/− 0.16 | 0.9 +/− 0.08 | 1.3 +/− 0.11 | 0.9 +/− 0.10 |
| PBEF | — | 217739_s_at | 1.3 +/− 0.11 | 1.8 +/− 0.19 | 1.0 +/− 0.09 | 1.4 +/− 0.07 | 1.0 +/− 0.08 |
| PBEF | — | 243296_at | 1.3 +/− 0.18 | 1.6 +/− 0.11 | 0.9 +/− 0.09 | 1.3 +/− 0.12 | 0.9 +/− 0.14 |
| PCDHA3 | 606309 | 210674_s_at | 0.8 +/− 0.07 | 0.7 +/− 0.02 | 1.0 +/− 0.16 | 0.9 +/− 0.07 | 1.0 +/− 0.07 |
| PCDHA3 | 606309 | 223435_s_at | 0.9 +/− 0.03 | 0.7 +/− 0.05 | 1.0 +/− 0.07 | 0.9 +/− 0.08 | 1.1 +/− 0.05 |
| PCTAIRE2B | — | 213361_at | 1.2 +/− 0.09 | 1.1 +/− 0.11 | 1.3 +/− 0.07 | 1.0 +/− 0.13 | 1.1 +/− 0.08 |
| [symbol] | | | | | | | |
| PDCD6IP | 608074 | 222394_at | 0.8 +/− 0.07 | 0.7 +/− 0.13 | 0.9 +/− 0.07 | 0.8 +/− 0.10 | 1.1 +/− 0.15 |
| PDE4DIP | 608117 | 214129_at | 0.8 +/− 0.16 | 0.7 +/− 0.08 | 1.0 +/− 0.05 | 0.9 +/− 0.14 | 1.0 +/− 0.03 |
| PDGFC | — | 218718_at | 0.8 +/− 0.09 | 0.7 +/− 0.07 | 0.9 +/− 0.07 | 1.1 +/− 0.13 | 1.0 +/− 0.08 |
| PDHA2 | 179061 | 214518_at | 0.9 +/− 0.09 | 1.3 +/− 0.20 | 0.9 +/− 0.16 | 0.8 +/− 0.14 | 1.1 +/− 0.09 |
| PDK4 | 602527 | 225207_at | 1.2 +/− 0.15 | 1.4 +/− 0.08 | 0.9 +/− 0.15 | 1.3 +/− 0.13 | 0.9 +/− 0.12 |
| PDLIM2 | — | 219165_at | 0.7 +/− 0.05 | 0.5 +/− 0.02 | 1.0 +/− 0.04 | 0.7 +/− 0.08 | 1.0 +/− 0.11 |
| PEA15 | 603434 | 200787_s_at | 0.8 +/− 0.04 | 0.7 +/− 0.05 | 0.9 +/− 0.08 | 0.8 +/− 0.05 | 1.0 +/− 0.07 |
| PECR | 605843 | 223619_x_at | 1.0 +/− 0.05 | 1.3 +/− 0.14 | 0.8 +/− 0.17 | 1.3 +/− 0.21 | 0.9 +/− 0.26 |
| PEG10 | — | 212094_at | 1.1 +/− 0.06 | 1.1 +/− 0.04 | 1.0 +/− 0.06 | 1.1 +/− 0.05 | 1.0 +/− 0.02 |
| PEN2 | 607632 | 218302_at | 1.2 +/− 0.10 | 1.3 +/− 0.06 | 1.0 +/− 0.08 | 1.2 +/− 0.03 | 1.0 +/− 0.11 |
| PEX11B | 603867 | 202658_at | 1.0 +/− 0.05 | 0.8 +/− 0.05 | 1.0 +/− 0.08 | 1.0 +/− 0.07 | 1.0 +/− 0.02 |
| PH-4 | — | 222125_s_at | 0.9 +/− 0.06 | 0.9 +/− 0.07 | 1.0 +/− 0.04 | 1.0 +/− 0.05 | 1.0 +/− 0.03 |
| PHF11 | 607796 | 221816_s_at | 1.9 +/− 0.08 | 2.1 +/− 0.11 | 1.6 +/− 0.07 | 1.7 +/− 0.27 | 1.4 +/− 0.08 |
| PHYH | 602026 | 203335_at | 0.9 +/− 0.03 | 0.8 +/− 0.02 | 1.0 +/− 0.04 | 0.9 +/− 0.02 | 1.0 +/− 0.06 |
| PIGF | 600153 | 212120_at | 0.9 +/− 0.04 | 0.8 +/− 0.02 | 1.0 +/− 0.06 | 0.9 +/− 0.03 | 1.0 +/− 0.02 |
| PIGM | — | 223470_at | 1.0 +/− 0.09 | 0.8 +/− 0.05 | 1.0 +/− 0.05 | 0.9 +/− 0.08 | 0.9 +/− 0.06 |
| PIP3AP | 606501 | 225232_at | 0.9 +/− 0.02 | 0.8 +/− 0.02 | 1.0 +/− 0.06 | 1.0 +/− 0.07 | 0.9 +/− 0.04 |
| PKP3 | 605561 | 209873_s_at | 0.9 +/− 0.06 | 0.7 +/− 0.05 | 1.0 +/− 0.07 | 0.8 +/− 0.08 | 1.0 +/− 0.10 |
| PLA2G12 | — | 228084_at | 0.7 +/− 0.08 | 0.6 +/− 0.07 | 0.9 +/− 0.09 | 0.8 +/− 0.07 | 1.0 +/− 0.14 |
| PLAUR | 173391 | 210845_s_at | 1.1 +/− 0.09 | 1.4 +/− 0.03 | 1.1 +/− 0.10 | 1.2 +/− 0.16 | 1.0 +/− 0.07 |
| PLAUR | 173391 | 211924_s_at | 1.2 +/− 0.13 | 1.5 +/− 0.11 | 1.0 +/− 0.16 | 1.0 +/− 0.10 | 1.0 +/− 0.07 |
| PLCB4 | 600810 | 203895_at | 0.8 +/− 0.05 | 0.7 +/− 0.06 | 1.0 +/− 0.07 | 0.8 +/− 0.08 | 1.0 +/− 0.11 |
| PLS1 | 602734 | 205190_at | 0.9 +/− 0.03 | 0.8 +/− 0.09 | 1.0 +/− 0.05 | 0.9 +/− 0.05 | 1.0 +/− 0.06 |
| PLSCR1 | 604170 | 202430_s_at | 1.8 +/− 0.19 | 1.7 +/− 0.27 | 2.3 +/− 0.17 | 1.3 +/− 0.14 | 1.7 +/− 0.17 |
| PLSCR1 | 604170 | 202446_s_at | 1.8 +/− 0.19 | 1.7 +/− 0.19 | 2.5 +/− 0.24 | 1.2 +/− 0.14 | 1.7 +/− 0.10 |
| PLSCR1 | 604170 | 241916_at | 1.6 +/− 0.23 | 1.4 +/− 0.25 | 1.9 +/− 0.12 | 1.2 +/− 0.14 | 1.4 +/− 0.16 |
| PMAIP1 | 604959 | 204285_s_at | 1.3 +/− 0.18 | 1.8 +/− 0.20 | 0.9 +/− 0.21 | 1.4 +/− 0.25 | 1.0 +/− 0.13 |
| PMAIP1 | 604959 | 204286_s_at | 1.2 +/− 0.10 | 1.7 +/− 0.31 | 0.9 +/− 0.10 | 1.4 +/− 0.24 | 0.9 +/− 0.22 |
| PMX1 | 167420 | 217226_s_at | 0.8 +/− 0.05 | 0.6 +/− 0.10 | 1.0 +/− 0.09 | 0.8 +/− 0.11 | 1.0 +/− 0.11 |
| PNPT1 | — | 225291_at | 1.2 +/− 0.09 | 1.1 +/− 0.13 | 1.4 +/− 0.07 | 1.1 +/− 0.12 | 1.2 +/− 0.03 |
| PP | 179030 | 217848_s_at | 1.2 +/− 0.04 | 1.3 +/− 0.04 | 1.1 +/− 0.09 | 1.1 +/− 0.09 | 1.1 +/− 0.04 |
| PP1665 | — | 32502_at | 0.7 +/− 0.08 | 0.6 +/− 0.03 | 0.9 +/− 0.09 | 0.8 +/− 0.04 | 1.0 +/− 0.11 |
| PPA2 | — | 1559496_at | 0.9 +/− 0.11 | 0.9 +/− 0.04 | 0.8 +/− 0.03 | 1.1 +/− 0.16 | 1.0 +/− 0.07 |
| PPP1R3C | 602999 | 204284_at | 0.8 +/− 0.04 | 0.6 +/− 0.09 | 0.9 +/− 0.08 | 0.8 +/− 0.08 | 0.9 +/− 0.08 |
| PRCP | 176785 | 201494_at | 0.9 +/− 0.03 | 0.9 +/− 0.07 | 1.0 +/− 0.04 | 0.9 +/− 0.04 | 1.1 +/− 0.07 |
| PRDX6 | 602316 | 200845_s_at | 1.1 +/− 0.04 | 1.2 +/− 0.02 | 1.0 +/− 0.02 | 1.1 +/− 0.04 | 1.0 +/− 0.05 |
| PRG1 | 177040 | 201858_s_at | 1.6 +/− 0.28 | 2.2 +/− 0.25 | 1.1 +/− 0.34 | 1.5 +/− 0.29 | 1.2 +/− 0.26 |
| PRG1 | 177040 | 201859_at | 1.2 +/− 0.07 | 1.5 +/− 0.05 | 1.0 +/− 0.08 | 1.2 +/− 0.11 | 1.0 +/− 0.07 |
| PRKCA | 176960 | 213093_at | 0.8 +/− 0.04 | 0.8 +/− 0.08 | 0.9 +/− 0.07 | 0.9 +/− 0.10 | 0.9 +/− 0.04 |
| PRKCM | 605435 | 205880_at | 0.9 +/− 0.08 | 0.7 +/− 0.11 | 0.8 +/− 0.15 | 0.9 +/− 0.06 | 1.1 +/− 0.08 |
| PRKR | 176871 | 204211_x_at | 1.4 +/− 0.08 | 1.3 +/− 0.08 | 1.7 +/− 0.35 | 1.1 +/− 0.15 | 1.4 +/− 0.14 |
| PRRG1 | 604428 | 205618_at | 0.9 +/− 0.07 | 1.1 +/− 0.06 | 0.9 +/− 0.09 | 1.1 +/− 0.11 | 0.8 +/− 0.09 |
| PRSS11 | 602194 | 201185_at | 0.7 +/− 0.09 | 0.5 +/− 0.19 | 1.1 +/− 0.16 | 0.7 +/− 0.07 | 0.9 +/− 0.14 |
| PRSS3 | — | 213421_x_at | 0.8 +/− 0.10 | 0.7 +/− 0.08 | 0.9 +/− 0.11 | 0.8 +/− 0.04 | 1.0 +/− 0.06 |
| PSMA2 | 176842 | 201316_at | 1.1 +/− 0.07 | 1.4 +/− 0.11 | 1.0 +/− 0.07 | 1.2 +/− 0.11 | 1.0 +/− 0.02 |
| PSMA2 | 176842 | 201317_s_at | 1.2 +/− 0.03 | 1.3 +/− 0.07 | 1.0 +/− 0.07 | 1.1 +/− 0.11 | 1.1 +/− 0.09 |
| PSMA3 | 176843 | 201532_at | 1.3 +/− 0.09 | 1.5 +/− 0.07 | 1.1 +/− 0.14 | 1.2 +/− 0.10 | 1.1 +/− 0.11 |
| PSMA4 | — | 203396_at | 1.2 +/− 0.06 | 1.3 +/− 0.07 | 1.1 +/− 0.08 | 1.1 +/− 0.09 | 1.1 +/− 0.04 |
| PSMA5 | 176844 | 201274_at | 1.3 +/− 0.07 | 1.5 +/− 0.10 | 1.0 +/− 0.08 | 1.2 +/− 0.09 | 1.0 +/− 0.10 |
| PSMB10 | 176847 | 202659_at | 5.1 +/− 0.36 | 8.5 +/− 0.52 | 1.2 +/− 0.11 | 5.0 +/− 0.40 | 1.1 +/− 0.10 |
| PSMB2 | 602175 | 200039_s_at | 1.3 +/− 0.06 | 1.4 +/− 0.06 | 1.1 +/− 0.06 | 1.2 +/− 0.02 | 1.1 +/− 0.05 |
| PSMB3 | 602176 | 201400_at | 1.2 +/− 0.06 | 1.3 +/− 0.08 | 1.1 +/− 0.09 | 1.1 +/− 0.05 | 1.1 +/− 0.07 |
| PSMB4 | 602177 | 202243_s_at | 1.1 +/− 0.07 | 1.3 +/− 0.05 | 1.0 +/− 0.11 | 1.1 +/− 0.07 | 1.0 +/− 0.06 |
| PSMB4 | 602177 | 202244_at | 1.1 +/− 0.08 | 1.2 +/− 0.07 | 1.0 +/− 0.08 | 1.0 +/− 0.03 | 1.0 +/− 0.05 |
| PSMB8 | 177046 | 209040_s_at | 10.1 +/− 0.53 | 14.3 +/− 0.48 | 2.1 +/− 0.37 | 9.3 +/− 0.76 | 1.4 +/− 0.24 |
| PSMB9 | 177045 | 204279_at | 11.1 +/− 0.49 | 15.3 +/− 0.57 | 1.8 +/− 0.16 | 10.5 +/− 1.01 | 1.3 +/− 0.19 |
| PSME1 | 600654 | 200814_at | 2.0 +/− 0.14 | 2.5 +/− 0.15 | 1.4 +/− 0.13 | 1.8 +/− 0.11 | 1.3 +/− 0.08 |
| PSME2 | 602161 | 201762_s_at | 2.7 +/− 0.13 | 3.1 +/− 0.16 | 1.4 +/− 0.12 | 2.4 +/− 0.13 | 1.3 +/− 0.13 |
| PSMF1 | — | 201052_s_at | 1.1 +/− 0.02 | 1.2 +/− 0.08 | 1.0 +/− 0.06 | 1.0 +/− 0.02 | 1.0 +/− 0.03 |
| PTPLA | — | 219654_at | 1.0 +/− 0.04 | 0.9 +/− 0.05 | 1.0 +/− 0.05 | 0.9 +/− 0.05 | 1.0 +/− 0.04 |
| PTPNS1 | 602461 | 202896_s_at | 0.8 +/− 0.02 | 0.7 +/− 0.10 | 1.0 +/− 0.17 | 0.7 +/− 0.07 | 0.9 +/− 0.08 |
| PTTG1IP | 603784 | 200677_at | 0.8 +/− 0.04 | 0.8 +/− 0.06 | 1.0 +/− 0.09 | 0.8 +/− 0.07 | 0.9 +/− 0.04 |
| RAB10 | — | 222981_s_at | 1.1 +/− 0.05 | 1.3 +/− 0.09 | 1.1 +/− 0.08 | 1.1 +/− 0.08 | 1.0 +/− 0.06 |
| RAB26 | 605455 | 50965_at | 0.9 +/− 0.10 | 0.7 +/− 0.10 | 1.0 +/− 0.04 | 0.9 +/− 0.12 | 1.0 +/− 0.09 |
| RAB27B | 603869 | 228708_at | 1.1 +/− 0.09 | 1.3 +/− 0.03 | 1.0 +/− 0.07 | 1.1 +/− 0.11 | 0.9 +/− 0.05 |
| RAB31 | 605694 | 217762_s_at | 1.2 +/− 0.14 | 1.4 +/− 0.14 | 1.0 +/− 0.11 | 1.2 +/− 0.07 | 1.0 +/− 0.03 |
| RAB31 | 605694 | 217763_s_at | 1.2 +/− 0.17 | 1.6 +/− 0.15 | 1.1 +/− 0.09 | 1.3 +/− 0.09 | 1.0 +/− 0.11 |
| RAB31 | 605694 | 217764_s_at | 1.2 +/− 0.04 | 1.5 +/− 0.08 | 1.0 +/− 0.07 | 1.3 +/− 0.08 | 1.1 +/− 0.01 |
| RABAC1 | 604925 | 203136_at | 0.9 +/− 0.09 | 0.9 +/− 0.06 | 1.0 +/− 0.05 | 0.8 +/− 0.04 | 1.0 +/− 0.03 |
| RABIF | 603417 | 204477_at | 0.8 +/− 0.07 | 0.7 +/− 0.12 | 1.0 +/− 0.10 | 0.8 +/− 0.11 | 1.1 +/− 0.21 |
| RARRES1 | 605090 | 206391_at | 1.8 +/− 0.54 | 2.2 +/− 0.30 | 0.9 +/− 0.45 | 1.8 +/− 0.32 | 1.2 +/− 0.33 |
| RARRES1 | 605090 | 206392_s_at | 2.8 +/− 0.91 | 4.1 +/− 0.69 | 1.2 +/− 0.57 | 2.7 +/− 0.69 | 1.1 +/− 0.30 |
| RARRES1 | 605090 | 221872_at | 1.8 +/− 0.38 | 2.8 +/− 0.39 | 1.0 +/− 0.19 | 2.0 +/− 0.21 | 1.0 +/− 0.12 |
| RARRES3 | 605092 | 204070_at | 30.8 +/− 4.31 | 49.8 +/− 3.06 | 1.6 +/− 0.09 | 30.3 +/− 2.77 | 1.3 +/− 0.14 |
| RBAF600 | — | 211950_at | 1.1 +/− 0.08 | 1.2 +/− 0.05 | 1.0 +/− 0.02 | 1.1 +/− 0.07 | 1.0 +/− 0.05 |
| RDH-E2 | — | 238017_at | 0.6 +/− 0.17 | 0.5 +/− 0.11 | 1.1 +/− 0.17 | 0.8 +/− 0.12 | 1.0 +/− 0.11 |
| REPIN1 | — | 225909_at | 1.4 +/− 0.08 | 1.7 +/− 0.17 | 1.3 +/− 0.20 | 1.4 +/− 0.20 | 1.3 +/− 0.06 |
| RGS2 | 600861 | 202388_at | 1.3 +/− 0.17 | 1.4 +/− 0.09 | 1.1 +/− 0.09 | 1.2 +/− 0.07 | 1.0 +/− 0.04 |
| RI58 | — | 203595_s_at | 1.7 +/− 0.18 | 1.9 +/− 0.26 | 1.8 +/− 0.26 | 1.5 +/− 0.14 | 1.4 +/− 0.12 |
| RIG-I | — | 222793_at | 1.6 +/− 0.14 | 1.8 +/− 0.10 | 2.0 +/− 0.09 | 1.2 +/− 0.01 | 1.4 +/− 0.24 |
| RIG-I | — | 242961_x_at | 1.4 +/− 0.12 | 1.4 +/− 0.15 | 1.6 +/− 0.17 | 1.2 +/− 0.13 | 1.3 +/− 0.11 |
| RREB1 | 602209 | 203704_s_at | 1.1 +/− 0.20 | 1.5 +/− 0.22 | 1.0 +/− 0.08 | 1.2 +/− 0.08 | 0.9 +/− 0.01 |
| S100A11 | 603114 | 200660_at | 1.1 +/− 0.07 | 1.2 +/− 0.06 | 1.0 +/− 0.06 | 1.0 +/− 0.06 | 1.0 +/− 0.06 |
| SAMHD1 | 606754 | 204502_at | 1.9 +/− 0.37 | 2.1 +/− 0.41 | 1.3 +/− 0.46 | 0.9 +/− 0.13 | 1.0 +/− 0.32 |
| SASH1 | 607955 | 226022_at | 0.9 +/− 0.09 | 0.8 +/− 0.03 | 0.9 +/− 0.05 | 0.8 +/− 0.05 | 1.0 +/− 0.01 |
| SAT | 313020 | 210592_s_at | 1.0 +/− 0.07 | 1.2 +/− 0.05 | 1.0 +/− 0.06 | 1.0 +/− 0.10 | 1.0 +/− 0.07 |
| SAT | 313020 | 213988_s_at | 1.1 +/− 0.14 | 1.5 +/− 0.27 | 0.9 +/− 0.07 | 1.1 +/− 0.20 | 1.0 +/− 0.10 |
| SCARA3 | 602728 | 219416_at | 0.7 +/− 0.11 | 0.6 +/− 0.13 | 1.2 +/− 0.18 | 0.8 +/− 0.19 | 1.1 +/− 0.04 |
| SCARA3 | 602728 | 223843_at | 0.8 +/− 0.10 | 0.6 +/− 0.08 | 1.0 +/− 0.07 | 0.8 +/− 0.18 | 1.0 +/− 0.09 |
| SCD4 | — | 224901_at | 0.9 +/− 0.09 | 0.8 +/− 0.08 | 1.0 +/− 0.03 | 0.8 +/− 0.03 | 1.1 +/− 0.04 |
| SCDGF-B | — | 219304_s_at | 0.9 +/− 0.04 | 0.8 +/− 0.05 | 1.0 +/− 0.03 | 0.9 +/− 0.05 | 0.9 +/− 0.04 |
| SCEL | 604112 | 206884_s_at | 0.8 +/− 0.08 | 0.7 +/− 0.07 | 1.0 +/− 0.10 | 0.8 +/− 0.11 | 0.9 +/− 0.10 |
| SCML1 | 300227 | 218793_s_at | 1.2 +/− 0.14 | 1.6 +/− 0.11 | 1.0 +/− 0.08 | 1.2 +/− 0.23 | 1.0 +/− 0.12 |
| SCOTIN | 607290 | 222986_s_at | 1.4 +/− 0.07 | 1.3 +/− 0.10 | 1.4 +/− 0.09 | 1.2 +/− 0.04 | 1.1 +/− 0.04 |
| SECTM1 | 602602 | 213716_s_at | 4.3 +/− 0.54 | 6.6 +/− 0.90 | 1.2 +/− 0.35 | 4.6 +/− 0.11 | 0.9 +/− 0.10 |
| SEMA3F | 601124 | 35666_at | 1.2 +/− 0.11 | 2.0 +/− 0.33 | 1.0 +/− 0.18 | 1.2 +/− 0.32 | 1.0 +/− 0.16 |
| SH3KBP1 | 300374 | 223082_at | 0.9 +/− 0.08 | 0.9 +/− 0.05 | 1.0 +/− 0.06 | 0.9 +/− 0.04 | 1.1 +/− 0.05 |
| SIAH1 | 602212 | 202981_x_at | 0.9 +/− 0.03 | 0.9 +/− 0.04 | 0.9 +/− 0.03 | 0.9 +/− 0.04 | 0.9 +/− 0.03 |
| SIAT8D | 602547 | 206925_at | 2.3 +/− 0.62 | 3.1 +/− 0.39 | 0.9 +/− 0.42 | 2.7 +/− 0.48 | 0.9 +/− 0.47 |
| SIAT8D | 602547 | 230261_at | 2.1 +/− 0.31 | 3.6 +/− 0.26 | 0.9 +/− 0.14 | 2.2 +/− 0.43 | 0.9 +/− 0.15 |
| SIAT8D | 602547 | 230836_at | 1.7 +/− 0.22 | 3.1 +/− 0.24 | 0.9 +/− 0.09 | 1.9 +/− 0.37 | 0.9 +/− 0.16 |
| SIAT8D | 602547 | 242943_at | 2.3 +/− 0.26 | 4.2 +/− 0.72 | 1.1 +/− 0.19 | 2.5 +/− 0.52 | 1.1 +/− 0.18 |
| SIX1 | 601205 | 228347_at | 1.1 +/− 0.15 | 1.7 +/− 0.08 | 1.0 +/− 0.09 | 1.2 +/− 0.17 | 1.0 +/− 0.06 |
| SLC16A6 | 603880 | 230748_at | 1.2 +/− 0.11 | 1.4 +/− 0.15 | 1.0 +/− 0.25 | 1.2 +/− 0.23 | 0.9 +/− 0.05 |
| SLC18A2 | 193001 | 230416_at | 0.9 +/− 0.04 | 0.8 +/− 0.03 | 0.9 +/− 0.04 | 1.0 +/− 0.06 | 0.9 +/− 0.03 |
| SLC1A1 | 133550 | 213664_at | 0.8 +/− 0.09 | 0.8 +/− 0.08 | 1.0 +/− 0.08 | 0.8 +/− 0.06 | 1.0 +/− 0.04 |
| SLC25A14 | 300242 | 211855_s_at | 0.9 +/− 0.07 | 0.8 +/− 0.05 | 0.9 +/− 0.05 | 0.9 +/− 0.09 | 1.1 +/− 0.05 |
| SLC25A22 | — | 218725_at | 1.1 +/− 0.08 | 1.3 +/− 0.04 | 1.0 +/− 0.10 | 1.1 +/− 0.14 | 0.9 +/− 0.14 |
| SLC30A1 | — | 212907_at | 1.3 +/− 0.13 | 1.4 +/− 0.06 | 1.0 +/− 0.10 | 1.2 +/− 0.13 | 0.9 +/− 0.08 |
| SLC30A1 | — | 228181_at | 1.3 +/− 0.12 | 1.4 +/− 0.09 | 1.0 +/− 0.15 | 1.4 +/− 0.12 | 0.9 +/− 0.11 |
| SLC3A1 | 104614 | 205799_s_at | 0.8 +/− 0.04 | 0.8 +/− 0.06 | 1.2 +/− 0.16 | 0.8 +/− 0.18 | 1.2 +/− 0.18 |
| SMG1 | 607032 | 224842_at | 1.1 +/− 0.04 | 1.2 +/− 0.03 | 1.0 +/− 0.06 | 1.1 +/− 0.05 | 1.0 +/− 0.09 |
| SNAP23 | 602534 | 209130_at | 0.9 +/− 0.02 | 0.8 +/− 0.03 | 0.9 +/− 0.03 | 0.9 +/− 0.02 | 1.0 +/− 0.03 |
| SNRPN | 182279 | 228370_at | 0.6 +/− 0.11 | 0.4 +/− 0.08 | 0.7 +/− 0.09 | 0.5 +/− 0.06 | 0.7 +/− 0.14 |
| SNX15 | 605964 | 202564_x_at | 1.1 +/− 0.04 | 1.2 +/− 0.05 | 1.0 +/− 0.05 | 1.1 +/− 0.04 | 1.0 +/− 0.03 |
| SOD2 | 147460 | 215223_s_at | 1.4 +/− 0.15 | 1.7 +/− 0.37 | 0.9 +/− 0.15 | 1.2 +/− 0.13 | 0.9 +/− 0.21 |
| SOD2 | 147460 | 216841_s_at | 1.4 +/− 0.09 | 2.0 +/− 0.19 | 1.0 +/− 0.06 | 1.4 +/− 0.24 | 1.0 +/− 0.05 |
| SORL1 | 602005 | 212560_at | 0.6 +/− 0.07 | 0.4 +/− 0.02 | 1.0 +/− 0.08 | 0.7 +/− 0.03 | 1.1 +/− 0.11 |
| SOX9 | 608160 | 202936_s_at | 0.8 +/− 0.06 | 0.6 +/− 0.07 | 0.9 +/− 0.03 | 0.8 +/− 0.07 | 0.9 +/− 0.10 |
| SP100 | 604585 | 202864_s_at | 1.6 +/− 0.07 | 1.7 +/− 0.18 | 1.5 +/− 0.17 | 1.2 +/− 0.11 | 1.3 +/− 0.07 |
| SP100 | 604585 | 210218_s_at | 1.9 +/− 0.45 | 2.3 +/− 0.43 | 1.8 +/− 0.31 | 1.4 +/− 0.19 | 1.4 +/− 0.13 |
| SP110 | 604457 | 208012_x_at | 1.6 +/− 0.28 | 1.9 +/− 0.31 | 1.8 +/− 0.36 | 1.6 +/− 0.12 | 1.5 +/− 0.09 |
| SP110 | 604457 | 209761_s_at | 1.7 +/− 0.18 | 2.1 +/− 0.18 | 1.9 +/− 0.28 | 1.5 +/− 0.24 | 1.6 +/− 0.10 |
| SP110 | 604457 | 209762_x_at | 1.9 +/− 0.11 | 2.2 +/− 0.07 | 2.0 +/− 0.12 | 1.5 +/− 0.10 | 1.6 +/− 0.17 |
| SP110 | 604457 | 223980_s_at | 2.0 +/− 0.18 | 2.6 +/− 0.22 | 2.4 +/− 0.09 | 1.5 +/− 0.14 | 1.5 +/− 0.23 |
| SPAG1 | 603395 | 210117_at | 0.8 +/− 0.07 | 0.6 +/− 0.03 | 0.8 +/− 0.05 | 0.8 +/− 0.02 | 0.8 +/− 0.12 |
| SPEC2 | — | 224709_s_at | 1.1 +/− 0.03 | 1.3 +/− 0.07 | 1.0 +/− 0.07 | 1.1 +/− 0.05 | 1.0 +/− 0.07 |
| SPINT2 | 605124 | 210715_s_at | 0.9 +/− 0.07 | 0.7 +/− 0.04 | 1.0 +/− 0.04 | 0.9 +/− 0.06 | 1.0 +/− 0.04 |
| SPRED2 | — | 212458_at | 0.9 +/− 0.05 | 0.8 +/− 0.07 | 1.0 +/− 0.06 | 0.9 +/− 0.08 | 1.0 +/− 0.08 |
| SPUVE | — | 202458_at | 0.9 +/− 0.04 | 0.9 +/− 0.05 | 1.0 +/− 0.08 | 0.9 +/− 0.03 | 1.1 +/− 0.08 |
| SQRDL | — | 217995_at | 1.5 +/− 0.11 | 2.0 +/− 0.17 | 1.1 +/− 0.17 | 1.6 +/− 0.10 | 1.1 +/− 0.12 |
| SSA1 | 109092 | 204804_at | 2.2 +/− 0.16 | 3.2 +/− 0.19 | 1.6 +/− 0.16 | 2.1 +/− 0.29 | 1.3 +/− 0.09 |
| SSSCA1 | 606044 | 212484_at | 0.8 +/− 0.05 | 0.7 +/− 0.12 | 1.2 +/− 0.11 | 0.9 +/− 0.09 | 1.0 +/− 0.18 |
| STARD10 | — | 223103_at | 0.8 +/− 0.09 | 0.6 +/− 0.06 | 1.1 +/− 0.17 | 0.8 +/− 0.11 | 1.1 +/− 0.04 |
| STARD10 | — | 232322_x_at | 0.7 +/− 0.11 | 0.6 +/− 0.14 | 0.8 +/− 0.12 | 0.6 +/− 0.14 | 0.8 +/− 0.14 |
| STAT1 | 600555 | 200887_s_at | 2.4 +/− 0.13 | 2.9 +/− 0.14 | 1.7 +/− 0.17 | 2.3 +/− 0.13 | 1.3 +/− 0.08 |
| STAT1 | 600555 | 209969_s_at | 5.7 +/− 0.71 | 7.6 +/− 0.26 | 2.4 +/− 0.56 | 5.4 +/− 0.91 | 1.5 +/− 0.27 |
| STAT2 | 600556 | 225636_at | 2.5 +/− 0.38 | 2.3 +/− 0.03 | 1.2 +/− 0.16 | 1.4 +/− 0.15 | 1.0 +/− 0.18 |
| STK6 | 602687 | 204092_s_at | 1.1 +/− 0.01 | 1.1 +/− 0.02 | 1.0 +/− 0.06 | 1.1 +/− 0.04 | 1.0 ± 0.03 |
| STXBP1 | 602926 | 202260_s_at | 0.9 +/− 0.10 | 0.6 +/− 0.08 | 1.1 +/− 0.09 | 0.8 +/− 0.09 | 1.0 ± 0.09 |
| SULF2 | — | 224724_at | 0.6 +/− 0.13 | 0.6 +/− 0.09 | 0.9 +/− 0.13 | 0.8 +/− 0.10 | 0.9 +/− 0.11 |
| SYNGR2 | 603926 | 201079_at | 0.9 +/− 0.07 | 0.8 +/− 0.08 | 1.1 +/− 0.06 | 0.9 +/− 0.06 | 1.0 +/− 0.04 |
| TACSTD1 | 185535 | 201839_s_at | 0.7 +/− 0.09 | 0.5 +/− 0.05 | 1.0 +/− 0.15 | 0.6 +/− 0.04 | 1.0 +/− 0.13 |
| TAP1 | 170260 | 1555852_at | 11.9 +/− 1.38 | 22.0 +/− 3.23 | 1.4 +/− 0.51 | 12.6 +/− 2.74 | 1.4 +/− 0.60 |
| TAP1 | 170260 | 202307_s_at | 4.8 +/− 0.83 | 8.4 +/− 0.45 | 1.5 +/− 0.17 | 4.6 +/− 0.75 | 1.1 +/− 0.09 |
| TAP2 | 170261 | 204769_s_at | 2.1 +/− 0.40 | 2.8 +/− 0.11 | 1.5 +/− 0.14 | 2.1 +/− 0.20 | 1.3 +/− 0.21 |
| TAP2 | 170261 | 225973_at | 2.8 +/− 0.39 | 4.2 +/− 0.54 | 1.4 +/− 0.06 | 3.1 +/− 0.40 | 1.2 +/− 0.11 |
| TAPBP | 601962 | 208829_at | 2.1 +/− 0.18 | 2.4 +/− 0.08 | 1.3 +/− 0.16 | 2.0 +/− 0.08 | 1.1 +/− 0.09 |
| TAPBP-R | 607081 | 218746_at | 5.4 +/− 0.69 | 6.7 +/− 0.59 | 1.5 +/− 0.15 | 5.3 +/− 0.52 | 1.5 +/− 0.28 |
| TAPBP-R | 607081 | 218747_s_at | 5.0 +/− 0.82 | 7.0 +/− 0.82 | 1.2 +/− 0.50 | 5.2 +/− 0.64 | 1.3 +/− 0.21 |
| TBC1D1 | — | 212350_at | 0.9 +/− 0.09 | 0.7 +/− 0.05 | 0.9 +/− 0.05 | 0.8 +/− 0.06 | 1.0 +/− 0.02 |
| TBC1D4 | — | 203387_s_at | 0.8 +/− 0.06 | 0.9 +/− 0.04 | 1.0 +/− 0.07 | 0.9 +/− 0.09 | 1.0 +/− 0.02 |
| TCF8 | 189909 | 212764_at | 1.1 +/− 0.03 | 1.3 +/− 0.10 | 0.9 +/− 0.07 | 1.1 +/− 0.15 | 1.0 +/− 0.06 |
| TCTE1L | 300302 | 203303_at | 0.8 +/− 0.03 | 0.7 +/− 0.06 | 1.0 +/− 0.05 | 0.9 +/− 0.06 | 1.0 +/− 0.06 |
| TEAD4 | 601714 | 41037_at | 1.1 +/− 0.09 | 1.2 +/− 0.04 | 1.0 +/− 0.07 | 1.1 +/− 0.09 | 0.9 +/− 0.08 |
| TES | 606085 | 202720_at | 0.9 +/− 0.03 | 0.8 +/− 0.05 | 1.0 +/− 0.08 | 0.9 +/− 0.05 | 1.0 +/− 0.06 |
| TFAP2C | 601602 | 205286_at | 0.8 +/− 0.05 | 0.8 +/− 0.03 | 1.0 +/− 0.08 | 0.8 +/− 0.08 | 1.0 +/− 0.04 |
| TGFA | 190170 | 205016_at | 0.8 +/− 0.14 | 0.7 +/− 0.09 | 1.1 +/− 0.14 | 0.8 +/− 0.08 | 1.0 +/− 0.07 |
| TGFBR1 | 190181 | 224793_s_at | 0.8 +/− 0.06 | 0.7 +/− 0.07 | 1.0 +/− 0.10 | 0.9 +/− 0.05 | 0.9 +/− 0.07 |
| TGM2 | 190196 | 211573_x_at | 1.6 +/− 0.20 | 1.6 +/− 0.17 | 1.1 +/− 0.18 | 1.4 +/− 0.29 | 1.1 +/− 0.16 |
| TIMP2 | 188825 | 224560_at | 0.7 +/− 0.15 | 0.5 +/− 0.04 | 1.0 +/− 0.15 | 0.7 +/− 0.06 | 0.9 +/− 0.07 |
| TIMP2 | 188825 | 231579_s_at | 0.8 +/− 0.10 | 0.7 +/− 0.02 | 1.1 +/− 0.13 | 0.8 +/− 0.10 | 1.0 +/− 0.09 |
| TIMP4 | 601915 | 206243_at | 0.6 +/− 0.07 | 0.4 +/− 0.10 | 1.0 +/− 0.21 | 0.6 +/− 0.08 | 1.0 +/− 0.14 |
| TIP-1 | — | 215464_s_at | 0.9 +/− 0.05 | 0.8 +/− 0.05 | 1.0 +/− 0.07 | 0.9 +/− 0.07 | 0.9 +/− 0.03 |
| TLR3 | 603029 | 206271_at | 4.7 +/− 0.56 | 5.9 +/− 0.97 | 1.3 +/− 0.11 | 4.8 +/− 1.00 | 1.1 +/− 0.18 |
| TMEM9 | — | 222988_s_at | 0.8 +/− 0.08 | 0.7 +/− 0.04 | 1.0 +/− 0.08 | 0.8 +/− 0.07 | 1.0 +/− 0.08 |
| TNC | 187380 | 201645_at | 1.4 +/− 0.11 | 1.8 +/− 0.36 | 0.8 +/− 0.39 | 1.4 +/− 0.30 | 1.0 +/− 0.24 |
| TNFRSF10D | 603614 | 227345_at | 0.6 +/− 0.11 | 0.5 +/− 0.07 | 0.6 +/− 0.10 | 0.6 +/− 0.07 | 0.7 +/− 0.18 |
| TNFRSF11A | 603499 | 238846_at | 0.8 +/− 0.14 | 0.5 +/− 0.05 | 1.1 +/− 0.17 | 0.8 +/− 0.10 | 1.1 +/− 0.10 |
| TNFRSF5 | 109535 | 35150_at | 1.2 +/− 0.14 | 1.7 +/− 0.23 | 1.2 +/− 0.14 | 1.3 +/− 0.12 | 1.2 +/− 0.17 |
| TNFRSF6 | 134637 | 204780_s_at | 1.1 +/− 0.09 | 1.4 +/− 0.14 | 1.0 +/− 0.09 | 1.1 +/− 0.09 | 1.0 +/− 0.10 |
| TNFRSF6 | 134637 | 204781_s_at | 1.0 +/− 0.07 | 1.2 +/− 0.10 | 0.8 +/− 0.06 | 1.0 +/− 0.14 | 0.8 +/− 0.10 |
| TNFSF10 | 603598 | 202687_s_at | 3.0 +/− 1.21 | 6.7 +/− 1.77 | 2.2 +/− 0.94 | 2.8 +/− 1.01 | 2.0 +/− 1.37 |
| TNFSF10 | 603598 | 202688_at | 2.1 +/− 0.53 | 4.0 +/− 1.39 | 1.5 +/− 0.91 | 1.8 +/− 0.73 | 1.1 +/− 0.72 |
| TNFSF13B | 603969 | 223501_at | 2.1 +/− 0.71 | 3.4 +/− 0.55 | 1.8 +/− 0.82 | 2.7 +/− 0.78 | 1.8 +/− 0.52 |
| TNFSF13B | 603969 | 223502_s_at | 1.8 +/− 0.28 | 3.0 +/− 0.51 | 1.2 +/− 0.41 | 1.7 +/− 0.31 | 1.3 +/− 0.23 |
| TPM1 | 191010 | 206116_s_at | 0.9 +/− 0.03 | 0.8 +/− 0.06 | 1.1 +/− 0.18 | 0.8 +/− 0.11 | 1.1 +/− 0.12 |
| TPM1 | 191010 | 210986_s_at | 0.9 +/− 0.08 | 0.9 +/− 0.02 | 1.1 +/− 0.02 | 0.9 +/− 0.05 | 1.0 +/− 0.07 |
| TReP-132 | — | 238520_at | 0.8 +/− 0.06 | 0.7 +/− 0.11 | 1.1 +/− 0.14 | 0.9 +/− 0.13 | 1.1 +/− 0.19 |
| TRIM14 | 606556 | 203148_s_at | 1.5 +/− 0.13 | 1.5 +/− 0.11 | 1.9 +/− 0.16 | 1.3 +/− 0.09 | 1.4 +/− 0.04 |
| TRIM22 | 606559 | 213293_s_at | 8.5 +/− 1.66 | 12.2 +/− 0.88 | 1.6 +/− 0.07 | 6.8 +/− 0.79 | 1.2 +/− 0.23 |
| TRIM29 | — | 202504_at | 0.7 +/− 0.09 | 0.7 +/− 0.13 | 0.9 +/− 0.07 | 0.9 +/− 0.09 | 0.9 +/− 0.03 |
| TRIM31 | — | 208170_s_at | 3.4 +/− 1.01 | 6.3 +/− 1.36 | 1.2 +/− 0.43 | 3.3 +/− 1.03 | 1.3 +/− 0.19 |
| TRIM31 | — | 210159_s_at | 1.8 +/− 0.03 | 2.6 +/− 0.71 | 1.1 +/− 0.24 | 1.7 +/− 0.21 | 1.1 +/− 0.21 |
| TRIM31 | — | 215444_s_at | 3.0 +/− 0.45 | 6.2 +/− 0.42 | 1.1 +/− 0.15 | 3.2 +/− 0.57 | 1.1 +/− 0.24 |
| TRIM38 | — | 203568_s_at | 1.3 +/− 0.16 | 1.6 +/− 0.17 | 1.1 +/− 0.13 | 1.3 +/− 0.10 | 1.1 +/− 0.07 |
| TRIM44 | — | 217760_at | 1.1 +/− 0.10 | 1.3 +/− 0.03 | 1.0 +/− 0.07 | 1.2 +/− 0.05 | 1.0 +/− 0.04 |
| TRIM6 | 607564 | 223599_at | 0.8 +/− 0.08 | 0.8 +/− 0.05 | 1.0 +/− 0.07 | 0.9 +/− 0.04 | 0.9 +/− 0.02 |
| TTC3 | 602259 | 208073_x_at | 0.8 +/− 0.08 | 0.7 +/− 0.11 | 0.9 +/− 0.10 | 0.8 +/− 0.07 | 0.9 +/− 0.05 |
| TTC3 | 602259 | 210645_s_at | 0.8 +/− 0.08 | 0.7 +/− 0.03 | 0.8 +/− 0.13 | 0.8 +/− 0.03 | 0.9 +/− 0.05 |
| TXNDC | — | 209476_at | 1.1 +/− 0.03 | 1.1 +/− 0.04 | 1.0 +/− 0.03 | 1.1 +/− 0.02 | 1.0 +/− 0.04 |
| UACA | — | 238868_at | 0.7 +/− 0.09 | 0.4 +/− 0.08 | 0.9 +/− 0.18 | 0.7 +/− 0.16 | 0.9 +/− 0.20 |
| UBD | 606050 | 205890_s_at | 35.0 +/− 11.92 | 144.0 +/− 45.82 | 0.7 +/− 0.10 | 48.2 +/− 20.47 | 1.0 +/− 0.16 |
| UBE2E1 | 602916 | 212519_at | 1.0 +/− 0.03 | 1.1 +/− 0.03 | 1.1 +/− 0.02 | 1.0 +/− 0.05 | 1.1 +/− 0.03 |
| UBE2L6 | 603890 | 201649_at | 11.0 +/− 0.63 | 15.4 +/− 0.39 | 2.0 +/− 0.32 | 10.0 +/− 0.73 | 1.4 +/− 0.11 |
| UBE3A | 601623 | 213128_s_at | 0.9 +/− 0.07 | 1.0 +/− 0.08 | 0.9 +/− 0.07 | 1.1 +/− 0.05 | 0.9 +/− 0.05 |
| UBL3 | 604711 | 201535_at | 0.8 +/− 0.04 | 0.7 +/− 0.07 | 0.9 +/− 0.12 | 0.9 +/− 0.02 | 0.9 +/− 0.08 |
| UCP2 | 601693 | 208998_at | 0.8 +/− 0.10 | 0.7 +/− 0.09 | 0.9 +/− 0.05 | 0.7 +/− 0.07 | 0.9 +/− 0.02 |
| ULBP2 | 605698 | 238542_at | 0.7 +/− 0.11 | 0.7 +/− 0.11 | 1.1 +/− 0.10 | 0.9 +/− 0.12 | 1.2 +/− 0.17 |
| UNC93B1 | 608204 | 220998_s_at | 1.0 +/− 0.04 | 1.0 +/− 0.07 | 1.3 +/− 0.13 | 1.0 +/− 0.08 | 1.0 +/− 0.12 |
| UPK3B | — | 206658_at | 0.8 +/− 0.07 | 0.6 +/− 0.05 | 1.0 +/− 0.12 | 0.8 +/− 0.11 | 1.0 +/− 0.13 |
| URB | — | 225242_s_at | 0.8 +/− 0.05 | 0.6 +/− 0.08 | 1.0 +/− 0.09 | 0.8 +/− 0.08 | 1.0 +/− 0.11 |
| USP18 | 607057 | 219211_at | 1.4 +/− 0.08 | 1.6 +/− 0.16 | 1.6 +/− 0.03 | 1.1 +/− 0.07 | 1.2 +/− 0.06 |
| USP31 | — | 229812_at | 0.7 +/− 0.04 | 0.8 +/− 0.07 | 0.9 +/− 0.04 | 0.8 +/− 0.08 | 0.9 +/− 0.09 |
| USP33 | — | 212513_s_at | 1.0 +/− 0.04 | 1.1 +/− 0.05 | 0.9 +/− 0.03 | 1.0 +/− 0.07 | 0.9 +/− 0.02 |
| USP42 | — | 226669_at | 1.1 +/− 0.11 | 1.4 +/− 0.05 | 1.1 +/− 0.06 | 1.2 +/− 0.11 | 1.0 +/− 0.07 |
| VAPA | 605703 | 208780_x_at | 1.1 +/− 0.04 | 1.1 +/− 0.03 | 1.0 +/− 0.02 | 1.0 +/− 0.03 | 1.0 +/− 0.04 |
| VDP | 603344 | 201832_s_at | 1.1 +/− 0.09 | 1.2 +/− 0.10 | 1.0 +/− 0.05 | 1.1 +/− 0.07 | 1.0 +/− 0.05 |
| VGCNL1 | — | 228608_at | 0.7 +/− 0.08 | 0.5 +/− 0.11 | 0.9 +/− 0.17 | 0.6 +/− 0.13 | 0.9 +/− 0.17 |
| WARS | 191050 | 200628_s_at | 4.0 +/− 0.33 | 10.3 +/− 1.12 | 1.1 +/− 0.08 | 4.1 +/− 0.29 | 1.1 +/− 0.05 |
| WARS | 191050 | 200629_at | 4.8 +/− 0.51 | 9.4 +/− 0.81 | 1.0 +/− 0.10 | 4.8 +/− 0.48 | 0.9 +/− 0.07 |
| WHSC1 | 602952 | 222777_s_at | 0.9 +/− 0.05 | 0.8 +/− 0.04 | 1.1 +/− 0.08 | 1.0 +/− 0.08 | 1.0 +/− 0.08 |
| WSB2 | — | 201760_s_at | 0.9 +/− 0.03 | 0.9 +/− 0.05 | 0.9 +/− 0.04 | 0.9 +/− 0.03 | 0.9 +/− 0.03 |
| ZAK | — | 225665_at | 0.8 +/− 0.05 | 0.8 +/− 0.10 | 0.9 +/− 0.06 | 0.8 +/− 0.05 | 0.9 +/− 0.03 |
| ZD52F10 | — | 226926_at | 0.7 +/− 0.09 | 0.6 +/− 0.05 | 1.0 +/− 0.08 | 0.7 +/− 0.10 | 1.0 +/− 0.04 |
| ZDHHC16 | — | 223212_at | 0.9 +/− 0.02 | 0.8 +/− 0.04 | 1.0 +/− 0.03 | 0.9 +/− 0.03 | 1.0 +/− 0.03 |
| ZFP30 | — | 207090_x_at | 0.9 +/− 0.05 | 0.7 +/− 0.05 | 1.0 +/− 0.06 | 0.8 +/− 0.02 | 1.0 +/− 0.09 |
| ZFP36 | 190700 | 201531_at | 1.2 +/− 0.06 | 1.5 +/− 0.38 | 0.9 +/− 0.21 | 1.4 +/− 0.19 | 0.7 +/− 0.19 |
| ZNF161 | 606747 | 202171_at | 0.9 +/− 0.02 | 0.8 +/− 0.02 | 0.9 +/− 0.03 | 0.9 +/− 0.02 | 1.0 +/− 0.02 |
| ZNF207 | 603428 | 228157_at | 1.0 +/− 0.08 | 1.0 +/− 0.08 | 1.0 +/− 0.03 | 1.1 +/− 0.07 | 0.8 +/− 0.04 |
| ZNF275 | — | 225382_at | 0.8 +/− 0.15 | 0.6 +/− 0.06 | 0.9 +/− 0.07 | 0.8 +/− 0.04 | 1.0 +/− 0.08 |
| ZNF275 | — | 225383_at | 0.9 +/− 0.06 | 0.8 +/− 0.05 | 1.0 +/− 0.05 | 0.9 +/− 0.10 | 1.0 +/− 0.06 |
| ZNF288 | 606025 | 235308_at | 1.2 +/− 0.05 | 1.3 +/− 0.14 | 1.1 +/− 0.06 | 1.2 +/− 0.11 | 1.1 +/− 0.07 |
| ZNF347 | — | 238819_at | 1.4 +/− 0.39 | 1.0 +/− 0.25 | 1.5 +/− 0.22 | 0.9 +/− 0.20 | 1.7 +/− 0.20 |
| ZNF9 | 116955/// | 206158_s_at | 1.1 +/− 0.02 | 1.2 +/− 0.05 | 1.1 +/− 0.06 | 1.1 +/− 0.07 | 1.1 +/− 0.04 |
| | [symbol] | | | | | | |
| ZNRD1 | 607525 | 223639_s_at | 1.1 +/− 0.11 | 1.4 +/− 0.11 | 1.0 +/− 0.08 | 1.1 +/− 0.11 | 1.0 +/− 0.12 |
| — | — | 1557116_at | 3.2 +/− 0.53 | 4.5 +/− 0.43 | 1.2 +/− 0.26 | 3.3 +/− 0.57 | 1.2 +/− 0.22 |
| — | — | 1557236_at | 8.0 +/− 1.51 | 14.5 +/− 2.56 | 1.8 +/− 0.32 | 8.6 +/− 1.80 | 1.3 +/− 0.39 |
| — | — | 1557779_at | 0.7 +/− 0.06 | 0.6 +/− 0.06 | 0.9 +/− 0.11 | 0.7 +/− 0.09 | 0.8 +/− 0.14 |
| — | — | 1558605_at | 0.3 +/− 0.24 | 0.4 +/− 0.23 | 0.9 +/− 0.18 | 0.6 +/− 0.19 | 1.0 +/− 0.14 |
| — | — | 1563075_s_at | 1.6 +/− 0.17 | 1.7 +/− 0.23 | 1.2 +/− 0.12 | 1.2 +/− 0.35 | 1.1 +/− 0.12 |
| — | — | 1564630_at | 0.8 +/− 0.06 | 0.7 +/− 0.04 | 0.9 +/− 0.05 | 0.8 +/− 0.06 | 1.0 +/− 0.10 |
| — | — | 1565635_at | 0.8 +/− 0.09 | 1.4 +/− 0.20 | 1.3 +/− 0.14 | 1.0 +/− 0.18 | 1.3 +/− 0.12 |
| — | — | 1568592_at | 1.7 +/− 0.13 | 2.1 +/− 0.23 | 1.1 +/− 0.29 | 1.7 +/− 0.38 | 1.0 +/− 0.12 |
| — | — | 1568609_s_at | 1.8 +/− 0.50 | 2.8 +/− 0.57 | 1.0 +/− 0.36 | 1.7 +/− 0.44 | 1.1 +/− 0.23 |
| — | — | 201042_at | 1.2 +/− 0.10 | 1.4 +/− 0.07 | 1.0 +/− 0.09 | 1.2 +/− 0.09 | 1.0 +/− 0.03 |
| — | — | 201278_at | 0.7 +/− 0.09 | 0.7 +/− 0.15 | 1.0 +/− 0.08 | 0.9 +/− 0.14 | 1.0 +/− 0.04 |
| — | — | 210987_x_at | 0.9 +/− 0.06 | 0.8 +/− 0.03 | 1.1 +/− 0.07 | 0.8 +/− 0.07 | 1.1 +/− 0.05 |
| — | — | 212067_s_at | 6.9 +/− 0.63 | 9.0 +/− 1.01 | 1.5 +/− 0.08 | 7.4 +/− 0.24 | 1.1 +/− 0.14 |
| — | — | 212509_s_at | 0.8 +/− 0.06 | 0.8 +/− 0.03 | 1.0 +/− 0.06 | 0.9 +/− 0.07 | 0.9 +/− 0.05 |
| — | — | 213158_at | 1.0 +/− 0.05 | 1.1 +/− 0.05 | 0.8 +/− 0.06 | 1.1 +/− 0.10 | 1.0 +/− 0.07 |
| — | — | 214808_at | 1.3 +/− 0.10 | 1.4 +/− 0.16 | 1.0 +/− 0.11 | 1.2 +/− 0.14 | 1.0 +/− 0.14 |
| — | — | 217436_x_at | 3.1 +/− 0.34 | 4.4 +/− 0.54 | 1.3 +/− 0.19 | 2.8 +/− 0.28 | 1.1 +/− 0.15 |
| — | — | 217604_at | 1.8 +/− 0.14 | 2.6 +/− 0.39 | 1.3 +/− 0.20 | 1.9 +/− 0.27 | 1.0 +/− 0.12 |
| — | — | 223326_s_at | 0.8 +/− 0.10 | 0.7 +/− 0.08 | 1.0 +/− 0.09 | 0.7 +/− 0.12 | 1.0 +/− 0.08 |
| — | — | 225199_at | 0.8 +/− 0.02 | 0.7 +/− 0.06 | 0.9 +/− 0.07 | 0.8 +/− 0.08 | 0.9 +/− 0.05 |
| — | — | 225685_at | 0.7 +/− 0.10 | 0.5 +/− 0.01 | 0.9 +/− 0.10 | 0.7 +/− 0.08 | 0.9 +/− 0.06 |
| — | — | 225950_at | 1.2 +/− 0.07 | 1.3 +/− 0.06 | 1.0 +/− 0.08 | 1.2 +/− 0.10 | 1.1 +/− 0.07 |
| — | — | 225996_at | 0.5 +/− 0.13 | 0.6 +/− 0.13 | 0.7 +/− 0.18 | 0.6 +/− 0.10 | 0.7 +/− 0.08 |
| — | — | 226040_at | 1.3 +/− 0.10 | 1.5 +/− 0.07 | 1.1 +/− 0.06 | 1.2 +/− 0.14 | 1.0 +/− 0.14 |
| — | — | 226885_at | 1.1 +/− 0.16 | 1.3 +/− 0.10 | 0.9 +/− 0.07 | 1.2 +/− 0.09 | 1.0 +/− 0.07 |
| — | — | 228120_at | 0.7 +/− 0.05 | 0.7 +/− 0.09 | 0.8 +/− 0.08 | 0.8 +/− 0.07 | 0.9 +/− 0.07 |
| — | — | 228217_s_at | 1.3 +/− 0.03 | 1.5 +/− 0.09 | 1.1 +/− 0.06 | 1.3 +/− 0.16 | 1.0 +/− 0.12 |
| — | — | 228391_at | 1.3 +/− 0.19 | 1.4 +/− 0.14 | 1.0 +/− 0.12 | 1.4 +/− 0.16 | 1.0 +/− 0.13 |
| — | — | 228567_at | 0.8 +/− 0.07 | 0.8 +/− 0.06 | 0.8 +/− 0.08 | 1.0 +/− 0.06 | 0.9 +/− 0.06 |
| — | — | 228675_at | 6.6 +/− 0.62 | 8.4 +/− 1.23 | 2.4 +/− 1.03 | 5.5 +/− 1.11 | 3.9 +/− 1.62 |
| — | — | 228805_at | 0.8 +/− 0.03 | 0.8 +/− 0.08 | 1.0 +/− 0.09 | 0.9 +/− 0.03 | 0.8 +/− 0.09 |
| — | — | 228959_at | 0.9 +/− 0.14 | 0.8 +/− 0.08 | 1.0 +/− 0.02 | 0.9 +/− 0.17 | 1.2 +/− 0.14 |
| — | — | 229088_at | 1.5 +/− 0.07 | 1.8 +/− 0.04 | 1.1 +/− 0.14 | 1.5 +/− 0.28 | 1.1 +/− 0.23 |
| — | — | 229349_at | 1.3 +/− 0.07 | 1.3 +/− 0.08 | 1.1 +/− 0.12 | 1.3 +/− 0.10 | 1.0 +/− 0.10 |
| — | — | 229544_at | 0.7 +/− 0.05 | 0.7 +/− 0.04 | 0.7 +/− 0.06 | 0.8 +/− 0.11 | 0.9 +/− 0.05 |
| — | — | 229872_s_at | 1.3 +/− 0.17 | 1.6 +/− 0.13 | 1.1 +/− 0.09 | 1.3 +/− 0.21 | 1.0 +/− 0.23 |
| — | — | 230314_at | 1.6 +/− 0.26 | 1.6 +/− 0.12 | 1.4 +/− 0.15 | 1.2 +/− 0.12 | 1.3 +/− 0.10 |
| — | — | 230405_at | 6.1 +/− 2.15 | 11.4 +/− 1.11 | 2.1 +/− 0.58 | 5.7 +/− 2.06 | 2.3 +/− 1.55 |
| — | — | 230487_at | 0.7 +/− 0.03 | 0.6 +/− 0.10 | 1.0 +/− 0.09 | 0.7 +/− 0.12 | 0.9 +/− 0.15 |
| — | — | 230766_at | 1.2 +/− 0.06 | 1.4 +/− 0.17 | 1.1 +/− 0.03 | 1.3 +/− 0.19 | 1.1 +/− 0.07 |
| — | — | 231270_at | 1.5 +/− 0.11 | 2.0 +/− 0.22 | 1.1 +/− 0.33 | 1.6 +/− 0.19 | 1.3 +/− 0.14 |
| — | — | 232270_at | 0.9 +/− 0.06 | 0.8 +/− 0.03 | 1.0 +/− 0.08 | 0.9 +/− 0.03 | 1.0 +/− 0.04 |
| — | — | 232375_at | 2.4 +/− 0.47 | 3.5 +/− 0.31 | 1.2 +/− 0.19 | 2.4 +/− 0.48 | 1.0 +/− 0.06 |
| — | — | 232615_at | 1.3 +/− 0.11 | 1.6 +/− 0.19 | 1.0 +/− 0.10 | 1.3 +/− 0.12 | 1.0 +/− 0.07 |
| — | — | 234260_at | 1.1 +/− 0.09 | 1.5 +/− 0.16 | 1.0 +/− 0.22 | 1.1 +/− 0.12 | 1.0 +/− 0.10 |
| — | — | 235157_at | 3.4 +/− 0.65 | 4.3 +/− 0.39 | 1.4 +/− 0.12 | 3.0 +/− 0.89 | 1.5 +/− 0.65 |
| — | — | 235230_at | 0.7 +/− 0.12 | 0.6 +/− 0.07 | 1.0 +/− 0.12 | 0.8 +/− 0.06 | 1.0 +/− 0.14 |
| — | — | 235421_at | 1.1 +/− 0.15 | 1.4 +/− 0.27 | 0.9 +/− 0.13 | 1.0 +/− 0.04 | 0.9 +/− 0.10 |
| — | — | 235964_x_at | 2.4 +/− 0.20 | 2.7 +/− 0.20 | 2.0 +/− 0.19 | 1.9 +/− 0.17 | 1.3 +/− 0.26 |
| — | — | 236191_at | 2.3 +/− 0.32 | 3.5 +/− 0.79 | 1.9 +/− 0.57 | 2.3 +/− 0.54 | 1.7 +/− 0.29 |
| — | — | 236513_at | 0.7 +/− 0.10 | 0.5 +/− 0.06 | 0.8 +/− 0.18 | 0.8 +/− 0.04 | 1.0 +/− 0.04 |
| — | — | 237435_at | 0.8 +/− 0.07 | 0.7 +/− 0.03 | 0.9 +/− 0.09 | 0.8 +/− 0.07 | 0.9 +/− 0.03 |
| — | — | 237879_at | 0.8 +/− 0.09 | 0.5 +/− 0.22 | 0.8 +/− 0.20 | 0.5 +/− 0.16 | 1.1 +/− 0.14 |
| — | — | 238191_at | 0.8 +/− 0.02 | 0.8 +/− 0.08 | 0.9 +/− 0.09 | 0.8 +/− 0.05 | 0.9 +/− 0.10 |
| — | — | 238327_at | 2.0 +/− 0.10 | 1.8 +/− 0.32 | 1.5 +/− 0.10 | 1.5 +/− 0.09 | 1.2 +/− 0.17 |
| — | — | 238513_at | 2.2 +/− 0.45 | 4.7 +/− 0.59 | 0.9 +/− 0.24 | 2.9 +/− 0.72 | 1.0 +/− 0.18 |
| — | — | 238712_at | 1.3 +/− 0.26 | 1.7 +/− 0.16 | 1.0 +/− 0.05 | 1.3 +/− 0.17 | 1.0 +/− 0.17 |
| — | — | 238725_at | 16.0 +/− 2.14 | 51.6 +/− 2.89 | 1.1 +/− 0.51 | 19.2 +/− 2.35 | 1.4 +/− 0.58 |
| — | — | 239218_at | 0.8 +/− 0.11 | 0.7 +/− 0.03 | 1.0 +/− 0.08 | 0.9 +/− 0.09 | 1.1 +/− 0.09 |
| — | — | 239587_at | 3.8 +/− 0.39 | 6.6 +/− 0.44 | 1.2 +/− 0.19 | 4.4 +/− 0.59 | 1.3 +/− 0.28 |
| — | — | 239784_at | 0.8 +/− 0.04 | 0.7 +/− 0.18 | 0.7 +/− 0.24 | 1.1 +/− 0.08 | 1.1 +/− 0.06 |
| — | — | 239973_at | 1.2 +/− 0.05 | 1.1 +/− 0.08 | 0.9 +/− 0.13 | 1.0 +/− 0.14 | 0.8 +/− 0.11 |
| — | — | 240939_x_at | 0.9 +/− 0.03 | 1.0 +/− 0.04 | 0.7 +/− 0.09 | 1.0 +/− 0.04 | 0.8 +/− 0.09 |
| — | — | 242181_at | 0.7 +/− 0.10 | 0.7 +/− 0.04 | 0.8 +/− 0.01 | 0.8 +/− 0.03 | 0.9 +/− 0.06 |
| — | — | 242245_at | 0.7 +/− 0.04 | 0.6 +/− 0.04 | 0.8 +/− 0.10 | 0.6 +/− 0.08 | 0.8 +/− 0.07 |
| — | — | 243271_at | 1.3 +/− 0.19 | 1.9 +/− 0.30 | 1.2 +/− 0.17 | 1.3 +/− 0.12 | 1.2 +/− 0.24 |
| — | — | 243788_at | 1.4 +/− 0.13 | 1.7 +/− 0.26 | 1.0 +/− 0.36 | 1.4 +/− 0.23 | 1.1 +/− 0.14 |
| — | — | 244387_at | 1.3 +/− 0.24 | 1.6 +/− 0.11 | 0.9 +/− 0.09 | 1.4 +/− 0.23 | 0.9 +/− 0.08 |
| — | — | 244628_at | 1.3 +/− 0.28 | 0.9 +/− 0.33 | 1.0 +/− 0.26 | 0.6 +/− 0.16 | 1.7 +/− 0.26 |
| — | — | 44702_at | 0.8 +/− 0.09 | 0.7 +/− 0.02 | 1.0 +/− 0.11 | 0.8 +/− 0.07 | 0.9 +/− 0.10 |
| — | — | 59697_at | 0.7 +/− 0.05 | 0.6 +/− 0.03 | 1.0 +/− 0.08 | 0.8 +/− 0.04 | 0.9 +/− 0.11 |
| — | — | 65588_at | 1.1 +/− 0.03 | 1.2 +/− 0.05 | 1.0 +/− 0.06 | 1.1 +/− 0.08 | 1.0 +/− 0.04 |

¹Expression level as ratio of signal following IFN treatment relative to untreated cells
²Online Mendelian Inheritance in Man reference number; www.ncbi.nlm.nih.gov/entrez/query.fcgi?CMD=search&DB=omim
³Affymetrix U133A microarray probe set ID

Global expression profiling following EC₅₀ exposure to IFN alfacon-1, IFN γ-1b, or a cocktail of the two allowed the antiviral state to be correlated with induction of a subset of IFN-stimulated genes. Genes identified through this analysis corresponded to classic antiviral components, IFN-stimulated genes more recently associated with direct antiviral functions, as well as expressed sequence tags (ESTs) and hypothetical proteins. The magnitude of these antiviral EC₅₀-correlated expression events in human hepatoma (Huh7) cells exposed to clinically relevant doses of IFN alfacon-1, IFN γ-1b, or a cocktail of the two was also probed because the standard of care for patients with chronic hepatitis C is type I IFN-containing regimens. Relative to type I IFNs used alone, addition of type II IFN resulted in enhanced expression not only of many of the genes correlated above with the direct antiviral state, but also of genes involved in: 1) antigen presentation to cytotoxic T lymphocytes; 2) macrophage, natural killer (NK), and T-helper 1 (Th1) cell recruitment and activation; 3) complement system function; 4) apoptosis; and 5) IFN-stimulated genes (ISGs) with unknown functions. Since many of these processes are correlated clinically with resolution of chronic HCV infection, the combined use of these IFNs could display a beneficial effect on viral clearance in patients infected with HCV and other viruses through enhancement of one of these processes or of the direct antiviral state.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A pharmaceutical admixture comprising an interferon-alpha (IFN-α) polypeptide; and an interferon-gamma (IFN-γ) polypeptide; wherein said composition is prepared by admixing (a) a first pharmaceutical composition comprising the IFN-α polypeptide and a first pharmaceutically acceptable carrier in a sterile aqueous solution; and (b) a second pharmaceutical composition comprising the IFN-γ polypeptide and a second pharmaceutically acceptable carrier in a sterile aqueous solution; and wherein the first and second pharmaceutically acceptable carriers are the same or different.

2. A pharmaceutical admixture comprising an interferon-alpha (IFN-α) polypeptide; and an interferon-gamma (IFN-γ) polypeptide; wherein said composition is prepared by admixing (a) a lyophilized preparation of a first pharmaceutical composition comprising the IFN-α polypeptide and a first pharmaceutically acceptable carrier; and (b) a second pharmaceutical composition comprising the IFN-γ polypeptide and a second pharmaceutically acceptable carrier in a sterile aqueous solution; and wherein the first and second pharmaceutically acceptable carriers are the same or different.

3. A pharmaceutical admixture comprising an interferon-alpha (IFN-α) polypeptide; and an interferon-gamma (IFN-γ) polypeptide; wherein said composition is prepared by admixing (a) a lyophilized preparation of a first pharmaceutical composition comprising the IFN-γ polypeptide and a first pharmaceutically acceptable carrier; and (b) a second pharmaceutical composition comprising the IFN-α polypeptide and a second pharmaceutically acceptable carrier in a sterile aqueous solution; and wherein the first and second pharmaceutically acceptable carriers are the same or different.

4. The pharmaceutical composition of any one of claims 1-3, wherein the composition is prepared within 24 hours of administering said composition to a patient.

5. The pharmaceutical composition of any one of claims 1-3, wherein the composition is stable for at least 24 hours at a temperature in a range of from about 4° C. to about 22° C.

6. The pharmaceutical composition of any one of claims 1-3, wherein the IFN-α polypeptide is pegylated.

7. The pharmaceutical composition of any one of claims 1-3, wherein the IFN-α polypeptide is Infergen® consensus IFN-α.

8. The pharmaceutical composition of any one of claims 1-3, wherein the IFN-γ polypeptide is Actimmune® IFN-γ.

9. A syringe comprising:
a) a first chamber pre-filled with a first pharmaceutical composition comprising an IFN-α polypeptide and a first pharmaceutically acceptable carrier in a sterile aqueous solution; and
b) a second chamber pre-filled with a second pharmaceutical composition comprising an IFN-γ polypeptide and a second pharmaceutically acceptable carrier in a sterile aqueous solution; wherein the first and second pharmaceutically acceptable carriers are the same or different; and wherein movement of a plunger through the syringe forces the contents of the first and second chambers into a common channel terminating in an aperture, thereby forming an admixture of the first and second pharmaceutical compositions prior to expulsion of the admixture through the aperture.

10. A drug delivery device comprising:
a) a first chamber pre-filled with a first pharmaceutical composition comprising a lyophilized IFN-α polypeptide and a first pharmaceutically acceptable carrier;
b) a second chamber pre-filled with a second pharmaceutical composition comprising an IFN-γ polypeptide and a second pharmaceutically acceptable carrier in a sterile aqueous solution;
wherein the first and second pharmaceutically acceptable carriers are the same or different; and
wherein the device dispenses drug by moving the contents of the second chamber into the first chamber, thereby solubilizing the lyophilized IFN-α polypeptide and forming an admixture of the first and second pharmaceutical compositions in aqueous solution.

11. A drug delivery device comprising:
a) a first chamber pre-filled with a first pharmaceutical composition comprising a lyophilized IFN-γ polypeptide and a first pharmaceutically acceptable carrier;
b) a second chamber pre-filled with a second pharmaceutical composition comprising an IFN-α polypeptide and a second pharmaceutically acceptable carrier in a sterile aqueous solution;
wherein the first and second pharmaceutically acceptable carriers are the same or different; and
wherein the device dispenses drug by moving the contents of the second chamber into the first chamber, thereby solubilizing the lyophilized IFN-γ polypeptide and forming an admixture of the first and second pharmaceutical compositions in aqueous solution.

12. A method of preparing a combination pharmaceutical admixture comprising an IFN-α polypeptide and an IFN-γ polypeptide, the method comprising:
admixing (a) a first pharmaceutical composition comprising the IFN-α polypeptide and a first pharmaceutically acceptable carrier in a sterile aqueous solution; and (b) a second pharmaceutical composition comprising the IFN-γ polypeptide and a second pharmaceutically acceptable carrier in a sterile aqueous solution, wherein the first and second pharmaceutically acceptable carrier are the same or different, and wherein said admixing is carried out less than 24 hours prior to administering the admixture to an individual in need thereof.

13. A method of treating a fibrotic disorder in an individual, the method comprising administering to an individual suffering from a fibrotic disease an amount of the admixture of claim 1 that is effective in the therapy or prophylaxis of the fibrotic disorder in the individual, wherein said admixing is carried out less than 24 hours prior to administering the admixture to the individual.

14. A method of treating cancer in an individual having a tumor, the method comprising administering to the individual an effective amount of the admixture of claim 1 to treat the cancer, wherein said admixing is carried out less than 24 hours prior to administering the admixture to the individual.

15. A method of treating a viral infection in an individual, the method comprising administering to an individual in need thereof an effective amount of the admixture of claim 1, to achieve a sustained viral response, wherein said admixing is carried out less than 24 hours prior to administering the admixture to the individual.

16. The method of claim 15, wherein the viral infection is a hepatitis C virus infection.

17. The method of any of claims 13-15, wherein the individual is a human.

18. A method of treating a hepatitis C virus (HCV) infection in a patient, the method comprising administering to the patient an effective amount of the admixture of claim 1 three times per week.

19. The method of claim 18, wherein the first pharmaceutical composition is interferon alfacon-1 and the second pharmaceutical composition is interferon gamma-1b.

20. A method of treating hepatitis C virus (HCV) infection in a patient, the method comprising administering to the patient (a) an amount of a first pharmaceutical composition comprising an IFN-α polypeptide and a first pharmaceutically acceptable carrier in a sterile aqueous solution delivered in a single dose or two or more divided doses per week; and (b) an amount of a second pharmaceutical composition comprising an IFN-γ polypeptide and a second pharmaceutically acceptable carrier in a sterile aqueous solution delivered in a single dose or in two or more divided doses per week; wherein the first and second pharmaceutically acceptable carriers are the same or different; and wherein at least one dose of the first pharmaceutical composition and one dose of the second pharmaceutical composition are admixed together prior to administration to the patient.

21. The method of claim 20, wherein the amount of the first pharmaceutical composition is delivered in three divided doses per week, wherein the amount of the second pharmaceutical composition is delivered in three divided doses per week, wherein for each week each of the three doses of the second pharmaceutical composition is paired with one of the three doses of the first pharmaceutical composition, and wherein in each dose pair the dose of the first pharmaceutical composition and the dose of the second pharmaceutical composition are admixed together prior to administration to the patient.

22. The method of claim 20, wherein the amount of the first pharmaceutical composition is delivered in seven divided doses per week, wherein the amount of the second pharmaceutical composition is delivered in three divided doses per week, wherein for each week each of the three doses of the second pharmaceutical composition is paired with one of the seven doses of the first pharmaceutical composition, and wherein in each dose pair the dose of the first pharmaceutical composition and the dose of the second pharmaceutical composition are admixed together prior to administration to the patient.

23. The method of claim 20, wherein the amount of the first pharmaceutical composition is delivered in a single dose per week, wherein the amount of the second pharmaceutical composition is delivered in three divided doses per week, wherein for each week one of the three doses of the second pharmaceutical composition is paired with the single dose of the first pharmaceutical composition, and wherein in the dose pair the dose of the first pharmaceutical composition and the dose of the second pharmaceutical composition are admixed together prior to administration to the patient.

24. The method of claim 20, wherein the amount of the first pharmaceutical composition is delivered in two divided doses per week, wherein the amount of the second pharmaceutical composition is delivered in three divided doses per week, wherein for each week each of the two doses of the first pharmaceutical composition is paired with one of the three doses of the second pharmaceutical composition, and wherein in each dose pair the dose of the first pharmaceutical composition and the dose of the second pharmaceutical composition are admixed together prior to administration to the patient.

25. The method of any of claims 20-24, wherein the IFN-α polypeptide is interferon alfa-2a, interferon alfa-2b, interferon alfa-2c, interferon alfacon-1, peginterferon alfa-2a, peginterferon alfa-2b or monoPEG (30 kD, linear)-ylated consensus IFN-α.

26. The method of any of claims 18-25, wherein the patient is a human.
